SEQUENCE LISTING (2 sequences)

SEQ ID NO: 1 - DNA, 1560 bp, Homo sapiens
tgcctctgcccacctcttctcttctgcttccatattacaggttcatcatgaatgagaaatgggacacaaactcttcagaaaactggcatcccatctggaatgtcaatgacacaaagcatcatctgtactcagatattaatattacctatgtgaactactatcttcaccagcctcaagtggcagcaatcttcattatttcctactttctgatcttctttttgtgcatgatgggaaatactgtggtttgctttattgtaatgaggaacaaacatatgcacacagtcactaatctcttcatcttaaacctggccataagtgatttactagttggcatattctgcatgcctataacactgctggacaatattatagcaggatggccatttggaaacacgatgtgcaagatcagtggattggtccagggaatatctgtcgcagcttcagtctttacgttagttgcaattgctgtagataggttccagtgtgtggtctacccttttaaaccaaagctcactatcaagacagcgtttgtcattattatgatcatctgggtcctagccatcaccattatgtctccatctgcagtaatgttacatgtgcaagaagaaaaatattaccgagtgagactcaactcccagaataaaaccagtccagtctactggtgccgggaagactggccaaatcaggaaatgaggaagatctacaccactgtgctgtttgccaacatctacctggctcccctctccctcattgtcatcatgtatggaaggattggaatttcactcttcagggctgcagttcctcacacaggcaggaagaaccaggagcagtggcacgtggtgtccaggaagaagcagaagatcattaagatgctcctgattgtggccctgctttttattctctcatggctgcccctgtggactctaatgatgctctcagactacgctgacctttctccaaatgaactgcagatcatcaacatctacatctacccttttgcacactggctggcattcggcaacagcagtgtcaatcccatcatttatggtttcttcaacgagaatttccgccgtggtttccaagaagctttccagctccagctctgccaaaaaagagcaaagcctatggaagcttatgccctaaaagctaaaagccatgtgctcataaacacatctaatcagcttgtccaggaatctacatttcaaaaccctcatggggaaaccttgctttataggaaaagtgctgaaaaaccccaacaggaattagtgatggaagaattaaaagaaactactaacagcagtgagatttaaaaagagctagtgtgataatcctaactctactacgcattatatatttaaatccattgctttttgtggctttgcacttcaaatttttcaaagaatgttctaaataaaacatttactgaaagccctctctggcaaaaaaattaaaaataaacaaaaatggtcataagatcataaacaatcttatgttgtataaaaatacgtagagtgacttagacatgtttgcatgaataaatatatttctagagaacagtta

SEQ ID NO: 2 - protein, 420 aa, Homo sapiens
MNEKWDTNSSENWHPIWNVNDTKHHLYSDINITYVNYYLHQPQVAAIFIISYFLIFFLCMMGNTVVCFIVMRNKHMHTVTNLFILNLAISDLLVGIFCMPITLLDNIIAGWPFGNTMCKISGLVQGISVAASVFTLVAIAVDRFQCVVYPFKPKLTIKTAFVIIMIIWVLAITIMSPSAVMLHVQEEKYYRVRLNSQNKTSPVYWCREDWPNQEMRKIYTTVLFANIYLAPLSLIVIMYGRIGISLFRAAVPHTGRKNQEQWHVVSRKKQKIIKMLLIVALLFILSWLPLWTLMMLSDYADLSPNELQIINIYIYPFAHWLAFGNSSVNPIIYGFFNENFRRGFQEAFQLQLCQKRAKPMEAYALKAKSHVLINTSNQLVQESTFQNPHGETLLYRKSAEKPQQELVMEELKETTNSSEI

(19) United States
(12) Patent Application Publication
(10) Pub. No.: US 2005/0136444 A1
(43) Pub. Date: Jun. 23, 2005

(54) Treating neuropathic pain with neuropeptide FF receptor 2 agonists

(76) Inventors: Audra L. Scully, Carlsbad, CA (US); Robert E. Davis, San Diego, CA (US); Kimberly E. Vanover, San Digo, CA (US); Luis Roberto Gardell, San Diego, CA (US); Jelveh Lameh, Encinitas, CA (US); Nicholas Michael Kelly, Bagsvaerd (DK); Fabio Bertozzi, Kobenhavn N (DK)

Correspondence Address: KNOBBE MARTENS OLSON & BEAR LLP, 2040 MAIN STREET, FOURTEENTH FLOOR, IRVINE, CA, 92614, US

(21) Appl. No.: 10/949,140

(22) Filed: Sep. 24, 2004

Related U.S. Application Data
(60) Provisional application No. 60/506,130, filed on Sep. 25, 2003.
(60) Provisional application No. 60/508,008, filed on Oct. 2, 2003.

(51) Int. Cl. C12Q 1/68; G01N 33/53; G01N 33/567

(52) U.S. Cl. 435/6; 435/7.2

(57) ABSTRACT

The invention described below relates to the discovery of the neuropeptide FF receptor subtype that mediates acute nociception and chronic neuropathic pain, compounds that selectively interact with this receptor subtype and methods for treating acute pain and chronic neuropathic pain.

### RELATED APPLICATIONS

The present application claims priority to U.S. Provisional Application Ser. No. 60/506,130, filed Sep. 25, 2003, by Scully, et al., and entitled “TREATING NEUROPATHIC PAIN WITH NEUROPEPTIDE FF RECEPTOR 2 AGONISTS,” and to U.S. Provisional Application Ser. No. 60/508,008, filed Oct. 2, 2003, by Scully, et al., and entitled “TREATING NEUROPATHIC PAIN WITH NEUROPEPTIDE FF RECEPTOR 2 AGONISTS,” both of which are incorporated by reference herein in their entirety, including any drawings.

### FIELD OF THE INVENTION

Aspects of the invention described below relate to methods for treating acute pain and chronic neuropathic pain using compounds that modulate the activity of the neuropeptide FF receptor subtype that mediates acute nociception and chronic neuropathic pain. Aspects of the invention also relates to compounds that selectively interact with this receptor subtype and methods of identifying said compounds.

### BACKGROUND OF THE INVENTION

Pain is a common human experience. It can range from acute to chronic forms; from mild and moderate to severe intensity. Over 65 million Americans suffer from painful conditions at any given time. The direct and indirect costs of pain exceeds $120 billion each year. Acute pain can be treated with opiates, anti-inflammatory agents and other analgesics; the choice of treatment usually depends on severity. The goal of this form of pain therapy is to block the transmission of sensory signal carrying pain signals and to control the affective response to nociceptive stimuli.

Drugs that are effective in treating inflammatory and acute pain usually are not effective in treating more chronic forms of pain. One form of chronic pain arises after damage to sensory nerves. The experience can range from mild increased sensitivity to touch or temperature to excruciating pain. This kind of pain is termed neuropathic since it is thought to involve an alteration in nervous system function or a reorganization of nervous system structure.

Neuropathic pain is both extremely difficult to manage clinically and remarkably common. Approximately 1.5% of the US population may suffer from neuropathic pain of one kind or another and this population could be larger if one includes many forms of back pain that are neurogenic in origin. Thus, neuropathic pain can be associated with nerve damage caused by trauma, disease, and chemical injury. Compounds that alleviate neuropathic pain may not be effective in treating acute pain (for example, gapapentin, tricylic antidepressants). The currently available treatments for neuropathic pain were not expressly designed to treat these kinds of pain and therefore, not surprisingly these drugs are not highly efficacious nor do these drugs work in all patients. Thus, there is pressing need for more effective and better-tolerated treatments for neuropathic pain.

### SUMMARY OF THE INVENTION

Disclosed herein are methods of identifying a compound effective in treating pain comprising contacting the compound with an NPFF2 receptor and determining whether the compound binds to the NPFF2 receptor.

Also disclosed herein are methods of screening for a compound able to affect one or more activities of an NPFF2 receptor comprising the steps of, a) contacting a recombinant cell with a test compound, wherein said recombinant cell comprises a recombinant nucleic acid expressing said NPFF2 receptor, provided that said cell does not have functional NPFF2 receptor expression from endogenous nucleic acid, and b) determining the ability of said test compound to affect one or more activities of said NPFF2 receptor, and comparing said ability with the ability of said test compound to affect said one or more NPFF2 receptor activities in a cell not comprising said recombinant nucleic acid; wherein said recombinant nucleic acid comprises an NPFF2 receptor nucleic acid selected from the group consisting of: a) nucleic acid of SEQ ID NO:1, b) nucleic acid encoding the amino acid SEQ ID NO:2, c) a derivative thereof encoding said NPFF2 receptor, wherein said derivative encodes a receptor having one or more activities of said NPFF2 receptor and comprises at least 20 contiguous nucleotides which can hybridize under stringent hybridization conditions to a complement of at least 20 contiguous nucleotides of SEQ ID NO:1.

Further disclosed herein are methods for treating acute and chronic pain of any type comprising contacting an organism with an effective amount of at least one compound wherein the compound activates an NPFF2 receptor subtype.

Also disclosed herein are methods of identifying a compound which is an agonist of an NPFF2 receptor, the method comprising: contacting said NPFF2 receptor with at least one test compound; and determining any increase in activity level of said NPFF2 receptor so as to identify a test compound which is an agonist of said NPFF2 receptor.

In addition, disclosed herein are methods of identifying a compound which is an agonist of an NPFF2 receptor, the method comprising: culturing cells which express the NPFF2 receptor; incubating the cells or a component extracted from the cells with at least one test compound; and determining any increase in activity of the NPPF2 receptor so as to identify a test compound which is an agonist of a NPFF receptor.

Disclosed herein are methods for treating pain comprising contacting an individual suffering from pain with an effective amount of at least one compound of Formula I, II, or III, as described herein, whereby one or more symptoms of the pain are reduced.

Further disclosed herein are methods of identifying a compound that alleviates hyperalgesia or allodynia in a subject, comprising: providing a subject suffering from hyperalgesia or allodynia with at least one compound of Formula I, II, or III, as described herein; and determining if said at least one compound reduces hyperalgesia or allodynia in the subject.

Also disclosed herein are methods of identifying a compound of Formula I, II, or III, which is an agonist of the NPFF2 receptor, the method comprising: contacting a NPFF2 receptor with at least one compound of Formula I, II, or III, as disclosed herein; and determining any increase in activity level of the NPFF2 receptor so as to identify a compound of Formula I, II, or III, which is an agonist of the NPFF2 receptor.

Disclosed herein are methods of identifying a compound which is an agonist of a NPFF2 receptor, the method comprising: culturing cells that express the NPFF2 receptor; incubating the cells with at least one compound of Formula I, II, or III, as disclosed herein; and determining any increase in activity of the NPPF2 receptor so as to identify a compound of Formula I, II, or III, which is an agonist of a NPFF receptor.

Further disclosed herein are methods of identifying a compound which is an agonist of a NPFF2 receptor, the method comprising: contacting the NPFF2 receptor with at least one compound of Formula I, II, or III, as disclosed herein; and determining whether said compound of Formula I, II, or III binds to said NPFF2 receptor.

Also disclosed are compounds of Formula I or II 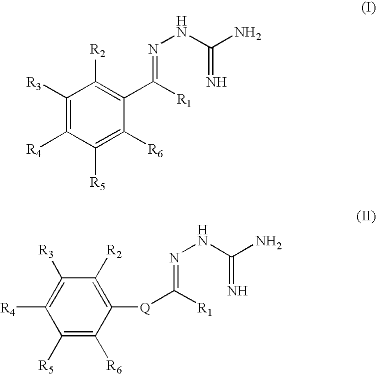
or a pharmaceutically acceptable salt, ester, amide, or prodrug thereof, as disclosed herein.

Further disclosed are compounds of Formula III 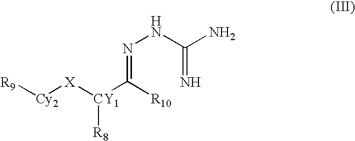
or a pharmaceutically acceptable salt, ester, amide, or prodrug thereof, as disclosed herein.

### BRIEF DESCRIPTION OF THE DRAWINGS

FIG. 1 is a bar graph comparing paw withdrawal latency periods.

FIG. 2 is a bar graph comparing tactile threshold levels.

FIG. 3 is a bar graph comparing tail withdrawal latency periods (in seconds).

FIG. 4 is a bar graph comparing the effect of selective FF2 receptor agonists on formalin-induced flinching. * Indicates p≦0.05 as compared to the formalin-injected vehicle-treated control group in each phase.

FIG. 5 is a bar graph comparing dose-dependent reversal of carrageenan-induced thermal hyperalgesia.

FIG. 6 is a bar graph showing dose-dependent reversal of L₅/L₆ SNL-induced tactile allodynia. * Indicates p≦0.05 as compared to the vehicle-treated controls.

FIG. 7 is a bar graph showing dose-dependent reversal of L₅/L₆ SNL-induced tactile allodynia using Compound 3099. * Indicates p≦0.05 as compared to the vehicle-treated controls.

FIG. 8 is a bar graph showing dose-dependent reversal of L₅/L₆ SNL-induced tactile allodynia using dPQRamide. * Indicates p≦0.05 as compared to the vehicle-treated controls.

### DETAILED DESCRIPTION OF THE PREFERRED EMBODIMENT

Neuropeptide FF (NPFF) is representative of a family of endogenously-expressed peptides that possess RF-amides at their C-termini and that act as neurotransmitters. NPFF is present in the central nervous system, particularly in the spinal cord, hypothalamus, thalamus and brainstem. One of the functions of this peptide is to modulate pain. In vivo studies suggest that NPFF can exert both pro- and anti-opioid effects in animal models of pain.

These seemingly opposing actions of NPFF could be mediated by actions at multiple receptors. Indeed, two G protein-coupled receptors are known to exist that are activated by NPFF. These receptors, termed NPFF1 and NPFF2, are differentially expressed throughout the body and across organisms. It is not known which of these two receptors mediates the actions of NPFF on various forms of pain. Anatomical studies showing NPFF2 binding sites in various brain regions including the spinal cord, dorsal root ganglion, spinal trigeminal nuclei and thalamus suggest that this receptor may mediate the nociceptive activity of NPFF in both forms of pain. However, without compounds that are selective for one NPFF receptor over the other, it is not possible to prove this assertion.

Therefore, compounds that bind to the NPFF2 receptor are prime candidates for further study as antinociceptive compounds. Identification of these compounds is of great interest in the art.

Compounds have been discovered that selectively activate the neuropeptide FF 2 (NPFF2) receptor relative to the neuropeptide FF 1 (NPFF1) and related receptors. Compounds that interact with the NPFF2 receptor subtype possess heretofore unappreciated analgesic activity and are effective treatments for acute and chronic pain. These observations have practical applications that support the use of NPFF2 receptor agonists in the treatment of acute pain and neuropathic pain caused by trauma, by diseases such as diabetes, herpes zoster (shingles), irritable bowel syndrome or late-stage cancer, or by chemical injury, for example, as an unintended consequence of drug therapies including the antiviral drugs.

Thus, the compounds and methods disclosed herein relate to the treatment of acute and chronic pain. Compounds selective for the NPFF2 receptor are disclosed. Methods for treating pain comprising contacting a subject with a pharmacologically active dose of a compound that interacts with the NPFF2 receptor subtype for the purpose of controlling pain without also causing unwanted and dose limiting side-effects are also disclosed.

Thus, in a first aspect, the present invention relates to a method of identifying a compound effective in treating pain comprising contacting the compound with an NPFF2 receptor and determining whether the compound binds to the NPFF2 receptor. The invention also relates to the use of an NPFF2 receptor in identifying compounds that bind to the NPFF2 receptor.

In another aspect, the present invention relates to a method of screening for a compound able to affect one or more activities of an NPFF2 receptor comprising the steps of,

a) contacting a recombinant cell with a test compound, wherein said recombinant cell comprises a recombinant nucleic acid expressing said NPFF2 receptor, provided that said cell does not have functional NPFF2 receptor expression from endogenous nucleic acid, and

b) determining the ability of said test compound to affect one or more activities of said NPFF2 receptor, and comparing said ability with the ability of said test compound to affect said one or more NPFF2 receptor activities in a cell not comprising said recombinant nucleic acid;

wherein said recombinant nucleic acid comprises an NPFF2 receptor nucleic acid selected from the group consisting of:

a) nucleic acid of SEQ ID NO:1,

b) nucleic acid encoding the amino acid SEQ ID NO:2,

c) a derivative thereof encoding said NPFF2 receptor, wherein said derivative encodes a receptor having one or more activities of said NPFF2 receptor and comprises at least 20 contiguous nucleotides which can hybridize under stringent hybridization conditions to a complement of SEQ ID NO:1.

In certain embodiments, the NPFF2 receptor nucleic acid encodes the amino acid sequence of a SEQ ID NO:2 derivative comprising at least 20 contiguous nucleotides which can hybridize under stringent hybridizations conditions to a complement of at least 20 contiguous nucleotides encoding the amino acid sequence of SEQ ID NO:2.

In some embodiments, the derivative comprises at least 50, at least 100, at least 150, at least 200, at least 250, at least 300, at least 350, at least 400, at least 450, at least 500, at least 600, at least 700, at least 800, at least 900, at least 1000, at least 1100, at least 1200, at least 1300, at least 1400, or at least 1500 contiguous nucleotides which can hybridize under stringent hybridizations conditions to a complement of contiguous nucleotides encoding the amino acid sequence of SEQ ID NO:2.

In another aspect, the present invention relates to a method for treating acute and chronic pain of any type comprising contacting an organism with an effective amount of at least one compound wherein the compound activates an NPFF2 receptor subtype.

In certain embodiments, the pain is associated with diabetes, viral infection, irritable bowel syndrome, amputation, cancer, or chemical injury.

In another aspect, the present invention relates to a method of identifying a compound which is an agonist of an NPFF2 receptor, the method comprising contacting said NPFF2 receptor with at least one test compound; and determining any increase in activity level of said NPFF2 receptor so as to identify a test compound which is an agonist of said NPFF2 receptor.

In certain embodiments, the identified agonist activates the NPFF2 but not the NPFF1 receptor. In other embodiments, the identified agonist is selective for the NPFF2 receptor.

In yet another aspect, the present invention relates to a method of identifying a compound which is an agonist of an NPFF2 receptor, the method comprising culturing cells which express the NPFF2 receptor; incubating the cells or a component extracted from the cells with at least one test compound; and determining any increase in activity of the NPPF2 receptor so as to identify a test compound which is an agonist of a NPFF receptor.

In certain embodiments, the cells of the above culturing step overexpress said NPFF2 receptor.

In another aspect, the present invention relates to a compound of Formula I or Formula II 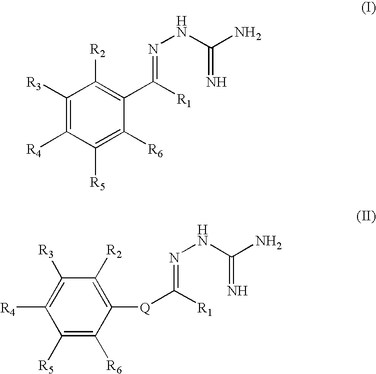
or a pharmaceutically acceptable salt, ester, amide, or prodrug thereof,
wherein

R₁ is selected from the group consisting of hydrogen, C₁-C₁₀ straight chained or branched alkyl, C₂-C₁₀ straight chained or branched alkenyl, C₂-C₁₀ straight chained or branched alkynyl, and C₃-C₁₀ cycloalkyl;

each of R₂, R₃, R₄, R₅ and R₆ is independently selected from the group consisting of hydrogen, C₁-C₁₀ straight chained or branched alkyl, C₂-C₁₀ straight chained or branched alkenyl, C₂-C₁₀ straight chained or branched alkynyl, C₃-C₁₀ cycloalkyl, substituted or unsubstituted aryl or heteroaryl, hydroxy, halogenated ether, nitro, amino, halogen, perhaloalkyl, —OR₇, —N(R₇)₂, —CN, —C(═Z)R₇, —C(═Z)OR₇, —C(═Z)N(R₇)₂, —N(R₇)—C(═Z)R₇, —N(R₇)—C(═Z)N(R₇)₂, —OC(═Z)R₇, and —SR₇
wherein Z is oxygen or sulfur; and wherein each R₇ is independently selected from the group consisting of hydrogen, C₁-C₁₀ straight chained or branched alkyl optionally substituted with an aryl or heteroaryl, C₂-C₁₀ straight chained or branched alkenyl optionally substituted with an aryl or heteroaryl, C₂-C₁₀ straight chained or branched alkynyl optionally substituted with an aryl or heteroaryl, C₃-C₁₀ cycloalkyl, C₅-C₁₀ cycloalkenyl, aryl, and heteroaryl; or

R₂ and R₃ and the carbons to which they are attached form a fused aryl, heteroaryl, C₅-C₁₀ carbocyclic or heterocyclic ring; or

R₃ and R₄ and the carbons to which they are attached form a fused aryl, heteroaryl, C₅-C₁₀ carbocyclic or heterocyclic ring; or

R₄ and R₅ and the carbons to which they are attached form a fused aryl, heteroaryl, C₅-C₁₀ carbocyclic or heterocyclic ring; or

R₅ and R₆ and the carbons to which they are attached form a fused aryl, heteroaryl, C₅-C₁₀ carbocyclic or heterocyclic ring; and

Q is selected from the group consisting of aryl, heteroaryl, C₅-C₁₀ carbocyclic or heterocyclic ring.

In another aspect, the present invention relates to a compound of Formula III 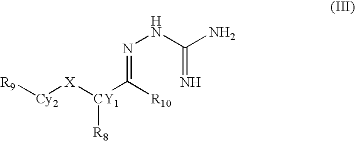
or a pharmaceutically acceptable salt, ester, amide, or prodrug thereof,
wherein

Cy₁ is selected from the group consisting of aryl, fused aryl, heteroaryl, fused heteroaryl, carbocyclic, cycloalkyl, fused heterocycle and heterocycle.

Cy₂ is selected from the group consisting of aryl, fused aryl, heteroaryl, fused heteroaryl, carbocyclic, cycloalkyl, fused heterocycle and heterocycle.

R₈ and R₉ are each present 0-6 times and are independently selected from the group consisting of hydrogen, C₁-C₈ straight chained or branched alkyl optionally substituted, C₂-C₈ straight chained or branched alkenyl optionally substituted, C₂-C₈ straight chained or branched alkynyl optionally substituted, C₃-C₈ cycloalkyl optionally substituted, carbocyclic optionally substituted, aryl optionally substituted, fused aryl optionally substituted, heteroaryl optionally substituted, fused heteroaryl optionally substituted, heterocycle optionally substituted, fused heterocycle optionally substituted, haloalkyl, halogen, —CN, —NO₂, —C(═Z)R₇, —C(═Z)OR₇, —C(═Z)N(R₇)₂, —N(R₇)₂, —N(R₇)—C(═Z)R₇, —N(R₇)—C(═Z)N(R₇)₂, —N(R₇)—S(═O)R₇, N(R₇)—S(═O)₂R₇, —OR₇, —OC(═Z)R₇, —SO₃H, —S(═O)₂N(R₇)₂, —S(═O)N(R₇)₂, —S(═O)₂R₇, —S(═O)R₇ and —SR₇,
wherein Z is oxygen or sulfur; and wherein each R₇ is as defined above;

R₁₀ is selected from the group consisting of hydrogen, C₁-C₈ straight chained or branched alkyl optionally substituted, C₂-C₈ straight chained or branched alkenyl optionally substituted, C₂-C₈ straight chained or branched alkynyl optionally substituted, C₃-C₈ cycloalkyl, aryl optionally substituted, fused aryl optionally substituted, heteroaryl optionally substituted, fused heteroaryl optionally substituted heterocycle optionally substituted, fused heterocycle optionally substituted.

X is either absent or selected from the group consisting of oxygen, sulfur, NR₇, ethylene optionally substituted, acetylene,
wherein R₇ is as defined above.

Some of the above substituents contain more than one “R” group, but the “R” groups are designated with identical numbers, for example, the group N(R₇)₂ has two R₇ groups. It is understood that the two “R” groups having the same number designation may be the same or may be different. Thus, for example, methylamine, dimethylamine, and methylpropylamine are all described by “N(R₇)₂.”

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

An “amide” is a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

Any amine, hydroxy, or carboxyl side chain on the compounds of the present invention can be esterified or amidified. The procedures and specific groups to be used to achieve this end is known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

The term “aromatic” refers to an aromatic group which has at least one ring having a conjugated pi electron system and includes both carbocyclic aryl (e.g., phenyl) and heterocyclic aryl groups (e.g., pyridine). The term includes monocyclic or fused-ring polycyclic (i.e., rings which share adjacent pairs of carbon atoms) groups. The term “carbocyclic” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon. The term “heteroaromatic” or “heteroaryl” refers to an aromatic group which contains at least one heterocyclic ring.

Examples of aryl ring include, but are not limited to, benzene, and substituted benzene, such as toluene, aniline, xylene, and the like.

Examples of fused aryl ring include, but are not limited to, naphthalene and substituted naphthalene, anthracene, and azulene.

Examples of heteroaryl ring include, but are not limited to, furan, thiophene, pyrrole, oxazole, thiazole, imidazole, pyrazole, isoxazole, isothiazole, oxadiazole, triazole, thiadiazole, pyridine, pyridazine, pyrimidine, pyrazine, triazine, 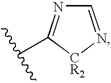 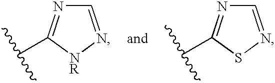
where R is as defined herein.

Examples of fused heteroaryl ring include, but are not limited to, indolizine, indole, isoindole, benzofuran, benzothiophene, indazole, benzimidazole, benzthiazole, purine, quinoline, isoquinoline, cinnoline, phthalazine, quinoxaline, quinoxaline, naphthyridine, pteridine, acridine, phenazine.

The term “heterocyclic” refers to a saturated or partially unsaturated ring with from three to fifteen units, in which at least one atom is different from carbon. The term includes monocyclic or fused-ring polycyclic (i.e., rings which share adjacent pairs of carbon atoms) groups.

Examples of heterocyclic ring include but are not limited to, pyrroline, pyrrolidine, dioxolane, imidazoline, imidazolidine, pyrazoline, pyrazolidine, pyran, piperidine, dioxane, mopholine, dithiane, thiomorpholine, piperazine.

Examples of fused heterocyclic ring include, but are not limited to, indoline, dihydrobenzofuran, dihydrobenzothiophene, carbazole, phenothiazine, phenoxazine, dihydroindole, dihydrobenzimidazole.

Examples of carbocyclic ring include, but are not limited to, indene, fluorene, adamantane, norbomane.

As used herein, the term “alkyl” refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds of the invention may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl.

The alkyl group may be substituted or unsubstituted. When substituted, the substituent group(s) is(are) one or more group(s) individually and independently selected from cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. Wherever a substituent is described as being “optionally substituted” that substitutent may be substituted with one of the above substituents.

The substituent “R” appearing by itself and without a number designation refers to a substituent selected from the group consisting of of alkyl, cycloalkyl, aryl, heteroaryl (bonded through a ring carbon) and heteroalicyclic (bonded through a ring carbon).

An “O-carboxy” group refers to a RC(═O)O— group, where R is as defined herein.

A “C-carboxy” group refers to a —C(═O)OR groups where R is as defined herein.

An “acetyl” group refers to a —C(═O)CH₃, group.

A “trihalomethanesulfonyl” group refers to a X₃CS(═O)₂— group where X is a halogen.

A “cyano” group refers to a -CN group.

An “isocyanato” group refers to a -NCO group.

A “thiocyanato” group refers to a -CNS group.

An “isothiocyanato” group refers to a -NCS group.

A “sulfinyl” group refers to a —S(═O)—R group, with R as defined herein.

A “S-sulfonamido” group refers to a —S(═O)₂NR, group, with R as defined herein.

A “N-sulfonamido” group refers to a RS(═O)₂NH— group with R as defined herein.

A “trihalomethanesulfonamido” group refers to a X₃CS(═O)₂NR— group with X and R as defined herein.

An “O-carbamyl” group refers to a —OC(═O)—NR, group with R as defined herein.

An “N-carbamyl” group refers to a ROC(═O)NH— group, with R as defined herein.

An “O-thiocarbamyl” group refers to a —OC(═S)—NR, group with R as defined herein.

An “N-thiocarbamyl” group refers to an ROC(═S)NH— group, with R as defined herein.

A “C-amido” group refers to a —C(═O)—NR₂ group with R as defined herein.

An “N-amido” group refers to a RC(═O)NH— group, with R as defined herein.

The term “perhaloalkyl” refers to an alkyl group where one or more of the hydrogen atoms are independently replaced by halogen atoms.

When two substituents and the carbons to which they are attached form a ring, it is meant that the following structure: 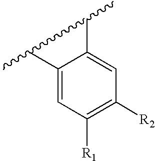
is representative of the following structure:

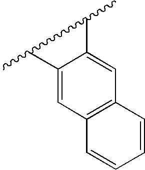

In the above example, R₁ and R₂ and the carbons to which they are attached form a six-membered aromatic ring.

Unless otherwise indicated, when a substituent is deemed to be “optionally subsituted,” it is meant that the subsitutent is a group that may be substituted with one or more group(s) individually and independently selected from cycloalkyl, aryl, heteroaryl, heterocyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof. The protecting groups that may form the protective derivatives of the above substituents are known to those of skill in the art and may be found in references such as Greene and Wuts, above.

In certain embodiments, R₁ in the compound of Formula I or II is hydrogen or C₁-C₁₀ straight chained alkyl. In some embodiments, R₁ is hydrogen or C₁-C₅ straight chained alkyl. In further embodiments, R₁ is selected from the group consisting of hydrogen, methyl, ethyl, n-propyl, isopropyl, n-butyl, sec-butyl, tert-butyl, n-pentyl, and isopentyl.

In some embodiments, R₂ in the compound of Formula I or II is selected from the group consisting of hydrogen, hydroxy, nitro, amino, halogen, —OR₇, and —N(R₇)₂, and wherein R₇ is hydrogen or C₁-C₁₀ straight chained alkyl. In certain embodiments, R₂ is selected from the group consisting of hydrogen, hydroxy, nitro, halogen, and —OR₇, and wherein R₇ is hydrogen or C₁-C₃ straight chained alkyl. In other embodiments, R₂ is selected from the group consisting of hydrogen, hydroxy, nitro, chloro, bromo, methoxy, and ethoxy.

In certain embodiments, R₃ in the compound of Formula I or II is selected from the group consisting of hydrogen, hydroxy, nitro, amino, halogen, —OR₇, and —N(R₇)₂, and wherein R₇ is hydrogen or C₁-C₁₀ straight chained alkyl. In some embodiments, R₃ is selected from the group consisting of hydrogen, hydroxy, nitro, halogen, and —OR₇, and wherein R₇ is hydrogen or C₁-C₃ straight chained alkyl. In other embodiments, R₃ is selected from the group consisting of hydrogen, nitro, chloro, and iodo.

Embodiments of the present invention include those in which R₄ in the compound of Formula I or II is selected from the group consisting of hydrogen, C₁-C₁₀ straight chained alkyl, hydroxy, nitro, amino, halogen, —OR₇, and —(NR₇)₂, and wherein each R₇ is independently C₁-C₁₀ straight chained or branched alkyl optionally substituted with an aryl or heteroaryl. In some embdoiments, R₄ is selected from the group consisting of hydrogen, C₁-C₃ straight chained alkyl, hydroxy, nitro, amino, halogen, —OR₇, and —(NR₇)₂, and wherein each R₇ is independently C₁-C₃ straight chained alkyl optionally substituted with an aryl. In yet other embodiments, R₄ is selected from the group consisting of hydrogen, methyl, ethyl, hydroxy, nitro, amino, chloro, fluoro, methoxy, ethoxy, methylamino, dimethylamino, diethylamino, and benzyloxy.

In further embodiments, R₅ in the compound of Formula I or II is selected from the group consisting of hydrogen, C₁-C₁₀ straight chained alkyl, hydroxy, nitro, amino, halogen, perhaloalkyl, —OR₇, and —(NR₇)₂, and wherein each R₇ is independently C₁-C₁₀ straight chained or branched alkyl optionally substituted with an aryl or heteroaryl. In other embodiments, R₅ is selected from the group consisting of hydrogen, C₁-C₃ straight chained alkyl, hydroxy, nitro, amino, halogen, perhaloalkyl, —OR₇, and —(NR₇)₂, and wherein each R₇ is independently C₁-C₃ straight chained alkyl. In certain embodiments, R₅ is selected from the group consisting of hydrogen, hydroxy, chloro, bromo, trifluoromethyl, and methoxy.

In some embodiments R₆ is hydrogen.

As mentioned above, in some embodiments R₂ and R₃ and the carbons to which they are attached form a fused aryl, heteroaryl, C₅-C₁₀ cyclic alkyl or heterocyclic alkyl ring. In some embodiments, the ring is a fused aryl ring, which may be a phenyl.

Some embodiments include those in which R₃ and R₄ and the carbons to which they are attached form a fused aryl, heteroaryl, C₅-C₁₀ cyclic alkyl or heterocyclic alkyl ring. The ring may be a fused heteroaryl ring, which may be a pyrrole.

In certain embodiments, R₄ and R₅ and the carbons to which they are attached form a fused aryl, heteroaryl, C₅-C₁₀ cyclic alkyl or heterocyclic alkyl ring. The ring may be a heterocyclic alkyl ring, which may be a 1,3-dioxolane.

In some embodiments, R₅ and R₆ and the carbons to which they are attached form a fused aryl, heteroaryl, C₅-C₁₀ cyclic alkyl or heterocyclic alkyl ring. The ring may be a fused aryl ring, which may be a phenyl.

In certain embodiments, Q is selected from the group consisting of optionally substituted benzene, toluene, aniline, xylene, naphthalene, azulene, furan, thiophene, pyrrole, pyrroline, pyrrolidine, oxazole, thiazole, imidazole, imidazoline, imidazolidine, pyrazole, pyrazoline, pyrazolidine, isoxazole, isothiazole, triazole, thiadiazole, pyran, pyridine, piperidine, morpholine, thiomorpholine, pyridazine, pyrimidine, pyrazine, piperazine, and triazine. In certain of these embodiments, Q is furan.

Those of skill in the art recognize that Q is doubly substituted: with an optionally substituted phenyl group and with the aminoguanidine group. It is further recognized that the two substitutions may be at different locations on Q. The two groups, thus, may be ortho, meta, or para to each other, i.e., they may be adjacent to each other on Q, or have one or more ring atoms separate the two ring atoms to which the two substituents are attached. All of the various structural isomers thus obtained are contemplated in the present invention.

In certain embodiments, the compound of Formula I is selected from the group consisting of 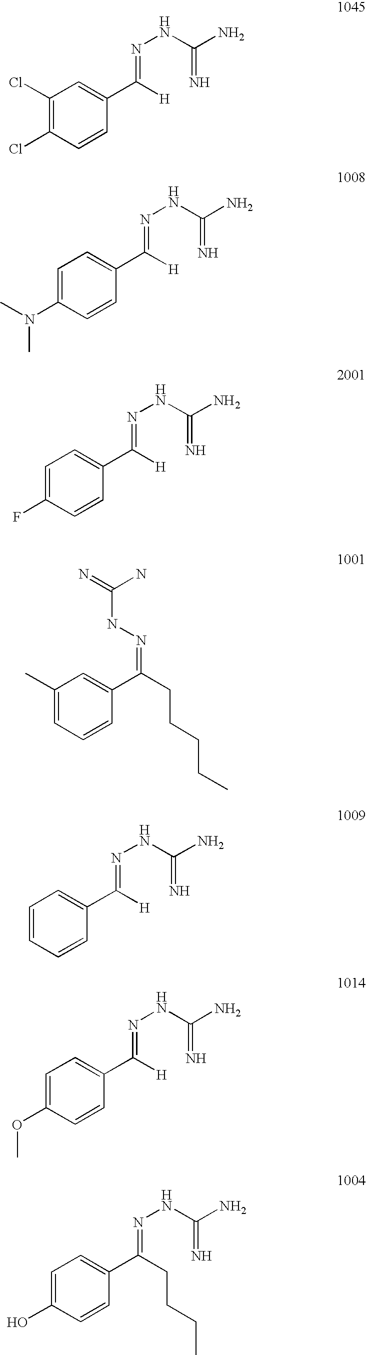 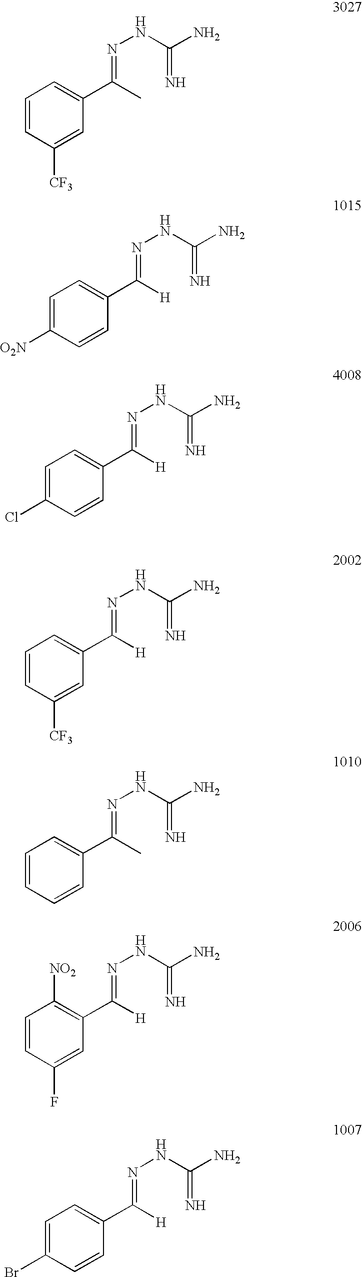 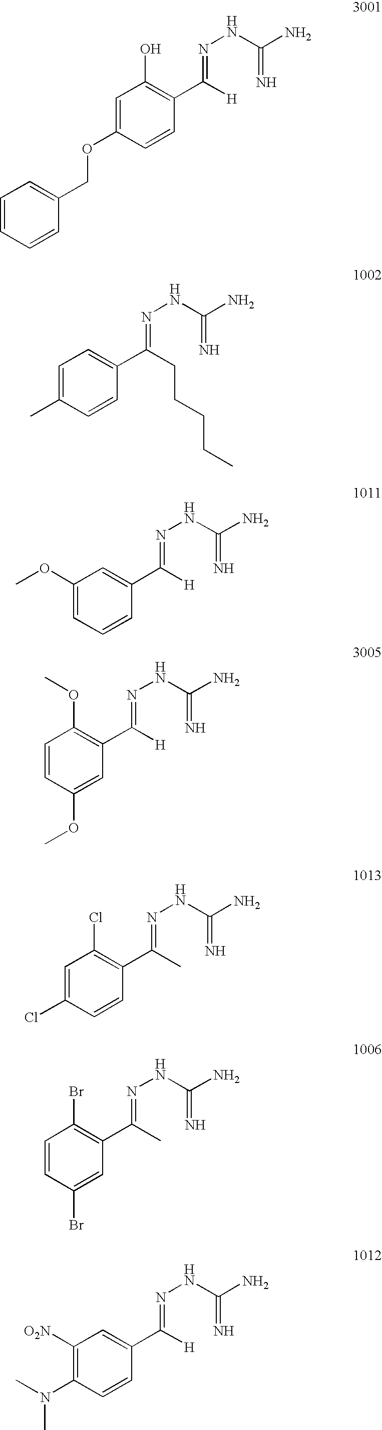 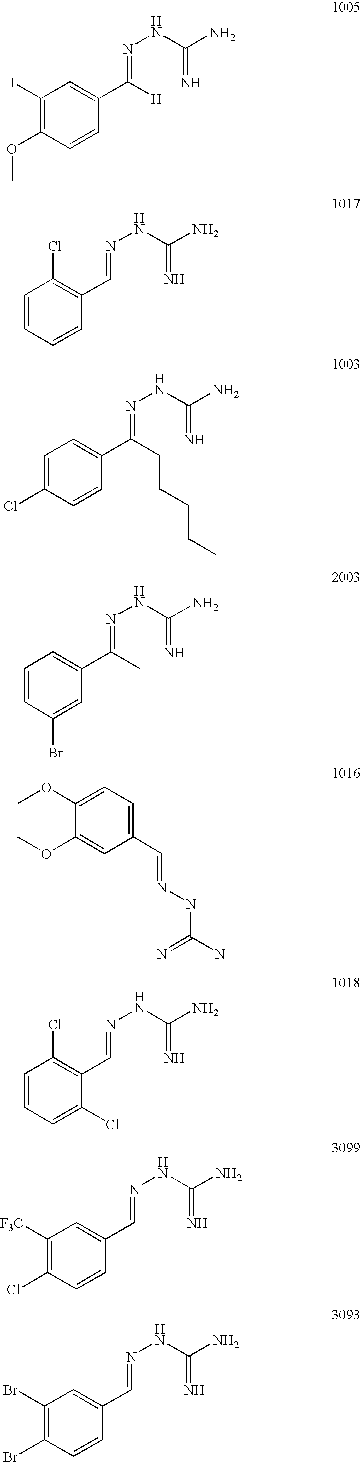 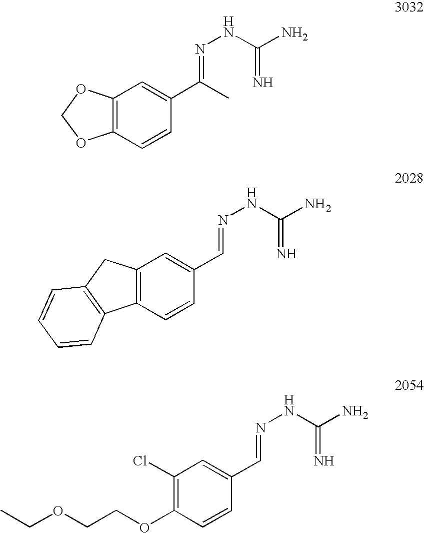
or a pharmaceutically acceptable salt or prodrug thereof.

In certain embodiments, the compound of Formula II is 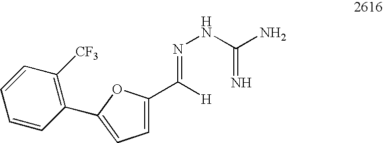
or a pharmaceutically acceptable salt or prodrug thereof.

In certain embodiments, the methods are also directed to methods for treating neuropathic pain. Particular preferred embodiments of compounds for use with the methods of this invention are represented by Compounds 1045, 3027, 3099, 1006, 1005, 3093, and 2616.

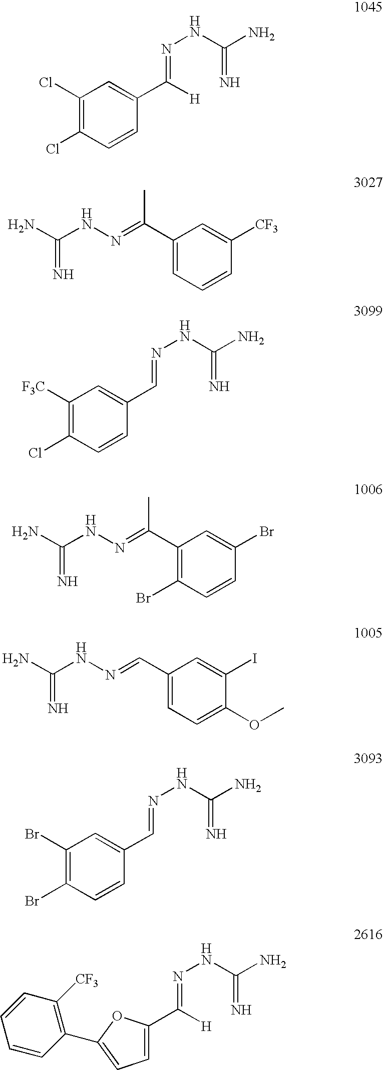

Certain of the compounds of the present invention may exist as stereoisomers including optical isomers. The invention includes all stereoisomers and both the racemic mixtures of such stereoisomers as well as the individual enantiomers that may be separated according to methods that are well known to those of ordinary skill in the art.

In another aspect, the present invention relates to a method for treating acute and chronic pain comprising identifying an individual in need thereof, and contacting said individual with an effective amount of at least one compound of Formula I, II, or III as defined herein, whereby one or more symptoms of the pain are reduced.

Another aspect of the present invention is the discovery that the disclosed NPFF2 compounds are specific agonists of the neuropeptide FF 2 receptor. Therefore, these agonists are expected to bind to the NPFF2 receptor and induce anti-hyperalgesic and anti-allodynic responses. The agonists of NPFF2 receptor described herein can be used to treat neuropathic pain.

Thus, in some embodiments, the compound of Formula I, II, or III activates the NPFF receptor. In certain embodiments, the compound may selectively activate the NPFF2 receptor subtype, but not NPFF1 receptor.

In certain embodiments, the pain treated by the methods of the present invention is associated with diabetes, viral infection, irritable bowel syndrome, amputation, cancer, inflammation or chemical injury. In other embodiments the pain is neuropathic pain.

In certain embodiments, the subject presents hyperalgesia. In some embodiments, the hyperalgesia is thermal hyperalgesia. In other embodiments, the subject presents allodynia. In some of these embodiments, the allodynia is tactile allodynia.

In another aspect, the present invention relates to a method of identifying a compound that alleviates hyperalgesia or allodynia in a subject, comprising identifying a subject suffering from hperalgesia or allodynia; providing the subject with at least one compound of Formula I, II, or III, as defined herein; and determining if said at least one compound reduces hyperalgesia or allodynia in the subject.

In yet another aspect, the present invention relates to a method of identifying a compound of Formula I, II, or III, which is an agonist of the NPFF2 receptor, the method comprising contacting a NPFF2 receptor with at least one compound of Formula I, II, or III, as defined herein; and determining any increase in activity level of the NPFF2 receptor so as to identify a compound of Formula I, II, or III, which is an agonist of the NPFF2 receptor.

In the context of present invention, an “agonist” is defined as a compound that increases the basal activity of a receptor (i.e. signal trans duction mediated by the receptor). An “antagonist” is defined as a compound which blocks the action of an agonist on a receptor. A “partial agonist” is defined as an agonist that displays limited, or less than complete, activity such that it fails to activate a receptor in vitro, functioning as an antagonist in vivo.

The term “subject” refers to an animal, preferably a mammal, and most preferably a human, who is the object of treatment, observation or experiment.

The term “therapeutically effective amount” is used to indicate an amount of an active compound, or pharmaceutical agent, that elicits the biological or medicinal response indicated. This response may occur in a tissue, system, animal or human that is being sought by a researcher, veterinarian, medical doctor or other clinician, and includes alleviation of the symptoms of the disease being treated.

In a further aspect, the present invention relates to a method of identifying a compound which is an agonist of a NPFF2 receptor, the method comprising culturing cells that express the NPFF2 receptor; incubating the cells with at least one compound of Formula I, II, or III, as defined herein; and determining any increase in activity of the NPPF2 receptor so as to identify a compound of Formula I which is an agonist of a NPFF receptor.

In yet another aspect, the present invention relates to a method of treating neuropathic or inflammatory pain in a subject comprising contacting the subject with a compound of Formula I, II, or III, where the compound acts as an antagonist or weak partial agonists at the NPFF1 receptor.

In a further aspect, the present invention relates to a method of treating neuropathic or inflammatory pain in a subject comprising contacting the subject with a combination of a compound of Formula I, II, or III, which acts as an antagonist or partial agonist to NPFF1 receptor, and another compound of Formula I, II, or III, which acts as a full agonist or a partial agonist to NPFF2 receptor.

In another aspect, the present invention relates to a method of treating neuropathic or inflammatory pain in a subject comprising contacting the subject with a compound of Formula I, II, or III, where the compound acts as both an NPFF2 agonist and an NPFF1 antagonist.

In another aspect, the present invention relates to a method of treating neuropathic or inflammatory pain in a subject comprising contacting the subject with a compound of Formula I, II, or III, where the compound acts as both an NPFF2 partial agonist and an NPFF1 antagonist.

In another aspect, the present invention relates to a method of treating neuropathic or inflammatory pain in a subject comprising contacting the subject with a compound of Formula I, II, or III, where the compound acts as both an NPFF2 partial agonist and an NPFF1 partial agonist.

In another aspect, the present invention relates to a pharmaceutical composition comprising a compound of Formula I, II, or III, as described above, and a physiologically acceptable carrier, diluent, or excipient, or a combination thereof.

The term “pharmaceutical composition” refers to a mixture of a compound of the invention with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly into the are of pain, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions of the present invention may be manufactured in a manner that is itself known, e.g., by means of conventional mixing, dissolving, granulating, dragee-making, levigating, emulsifying, encapsulating, entrapping or tabletting processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions of the present invention can be chosen by the individual physician in view of the patient's condition. (See e.g., Fingl et al. 1975, in “The Pharmacological Basis of Therapeutics”, Ch. 1 p. 1). Typically, the dose range of the composition administered to the patient can be from about 0.5 to 1000 mg/kg of the patient's body weight, or 1 to 500 mg/kg, or 10 to 500 mg/kg, or 50 to 100 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the present invention will use those same dosages, or dosages that are between about 0.1% and 500%, or between about 25% and 250%, or between 50% and 100% of the established human dosage. Where no human dosage is established, as will be the case for newly-discovered pharmaceutical compounds, a suitable human dosage can be inferred from ED₅₀ or BD₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions of the present invention or a pharmaceutically acceptable salt thereof calculated as the free base, the composition being administered 1 to 4 times per day. Alternatively the compositions of the invention may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. Suitably the compounds will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

It will be understood by those of skill in the art that numerous and various modifications can be made without departing from the spirit of the present invention. Therefore, it should be clearly understood that the forms of the present invention are illustrative only and are not intended to limit the scope of the present invention.

### Experimental Details

Synthesis of Chemical Compounds

Scheme 1 below is a representative synthetic scheme for the synthesis of imonoguanidines:

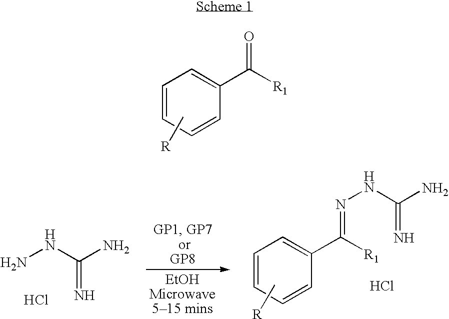

Alternative conditions can be used us shown in scheme 2 below:

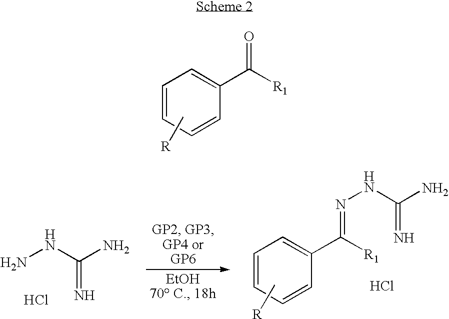

In some embodiments, an initial alkylation step is required prior to forming the imino guandidine group, as shown in scheme 3 below: 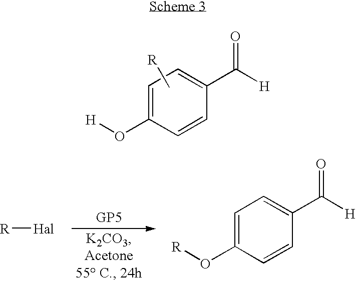
General Methods

96% ethanol was used and solvents were used as purchased. ¹H NMR spectra were recorded at 400 MHz on a Varian XL spectrometer. Chemical shifts are reported in parts per million (ppm) and referenced with respect to the residual (i.e. CHCl₃, CH₃OH) proton of the deuterated solvent. Splitting paterns are designated as: s=singlet, d=doublet, t=triplet, q=quartet, br.=broad, m=multiplet. Thin-layer chromatography (TLC) was carried out on aluminium sheets precoated with silica gel 60F₂₅₄. Flash column chromatography was performed on an Isco CombiFlash SQ16x using the methods described below. Microwave reactions were carried out using a Smith Creator from Personal Chemistry.

Analytical HPLC, Ammonium Acetate Buffer (ZMD)

System: Waters LC/ZMD instrument consisting of 600E Gradient Pump, 2700 Sample Manager, 996 Photodiode Array Detector and Electrospray Ionization Interface.

Column: Reversed phase column (Xterra® MS C₁₈ 5 μm, 50×4.6 mm ID).

Mobile Phase: Acetonitrile/10 mM aqueous Ammonium acetate.

Program: 17 min. gradient program starting at 10% Acetonitrile, over 10 min. to 100% Acetonitrile, hold for 1 min., over 0.5 min. to 10% Acetonitrile, hold for 5.5 min. The flow rate was 1 mL/min.

Analytical HPLC, Ammonium Acetate Buffer (ZQ)

System: Waters Alliance HT/ZQ2000 instrument consisting of 2795 Separation Module, 996 Photodiode Array Detector and Electrospray Ionization Interface.

Column: Reversed phase column (Xterra® MS C₁₈ 3.5 μm, 30×4.6 mm ID) with a guard column cartridge system.

Mobile Phase: Acetonitrile/10 mM aqueous Ammonium acetate.

Program: 11 min. gradient program starting at 10% Acetonitrile, over 7 min. to 90% Acetonitrile, over 0.5 min. to 10% Acetonitrile, hold for 3 min. The flow rate was 1 mL/min.

Analytical HPLC, Ammonium Bicarbonate Buffer (ZMD)

System: Waters LC/ZMD instrument consisting of 600E Gradient Pump, 2700 Sample Manager, 996 Photodiode Array Detector and Electrospray Ionization Interface.

Column: Reversed phase column (Xterra® MS C₁₈ 5 μm, 50×4.6 mm ID).

Mobile Phase: Acetonitrile/5 mM aqueous Ammonium Bicarbonate (adjusted to pH 9.5).

Program: 17 min. gradient program starting. at 10% Acetonitrile, over 10 min. to 100% Acetonitrile, hold for 1 min., over 0.5 min. to 10% Acetonitrile, hold for 5.5 min. The flow rate was 1 mL/min.

Preparative LC/MS Method

System: Waters LC/ZMD instrument. A set-up with a 600E Gradient Pump, 2700 Sample Manager, 996 Photodiode Array Detector and Electrospray Ionization Interface.

Column: Reversed phase column (Xterra® Prep MS C₁₈ 5 μm, 19×100 mm).

Mobile Phase: Acetonitrile/10 mM aqueous Ammonium acetate.

Program: A 12 min. gradient program starting at 30% Acetonitrile, over 8.5 min. to 100% Acetonitrile, over 0.5 min. to 30% Acetonitrile, hold for 0.5 min. The flow rate was 17 mL/min.

Preparative HPLC Method

System: Waters Prep4000 instrument. A set-up with a 4000 Prep Pump, Prep LC Controller, 2487 Dual Absorbance Detector.

Column: Semi-preparative column (Phenomenex® Luna C₁₈ 5 μm, 21.1×250 mm).

Mobile Phase: Acetonitrile/25 mM aqueous Ammonium acetate.

Program: A 45 min. gradient program starting at 10% Acetonitrile, hold for 5 min., over 30 min. to 80% Acetonitrile, hold for 10 min. The flow rate was 20 mL/min.

CombiFlash Method 1 (CF1)

The sample was dry loaded onto celite then purified on the CombiFlash using a 4 g silica column and eluting with EtOAc (3 min), 0-20% MeOH in EtOAc (25 min) then 20% MeOH in EtOAc (15 min) at 15 mL/min.

CombiFlash Method 2 (CF2)

The sample was dry loaded onto celite then purified on the CombiFlash using a 4 g silica column and eluting with heptane (1 min), 0-10% EtOAc in heptane (30 min), 10-15% EtOAc in heptane (10 min) then 15% EtOAc in heptane (5 min) at 16 mL/min.

CombiFlash Method 3 (CF3)

The sample was dry loaded onto celite then purified on the CombiFlash using a 4 g silica column and eluting with heptane (3 min), 0-25% EtOAc in heptane (25 min) then 25% EtOAc in heptane (8 min) at 15 mL/min.

CombiFlash Method 4 (CF4)

The sample was dry loaded onto celite then purified on the CombiFlash using a 4 g silica column and eluting with heptane (3 min), 0-15% EtOAc in heptane (25 min) then 15% EtOAc in heptane (10 min) at 15 mL/min.

CombiFlash Method 5 (CF5)

The sample was dry loaded onto celite then purified on the CombiFlash using a 4 g silica column and eluting with heptane (3 min), 0-10% EtOAc in heptane (25 min) then 10% EtOAc in heptane (8 min) at 15 mL/min.

CombiFlash Method 6 (CF6)

The sample was dry loaded onto celite then purified on the CombiFlash using a 10 g silica column and eluting with DCM (15 min), 0-10% MeOH in DCM (40 min) then 10% MeOH in DCM (10 min) at 15 mL/min.

General Procedure 1 (GP1)

The aldehyde or ketone (5.0 mmol) and aminoguanidine nitrate (5.0 mmol, 696 mg) in EtOH (3 mL) were heated in a microwave at 120° C. (aldehyde) or 160° C. (ketone) for 10 minutes then cooled to room temperature. MeOH (20 mL) then HCl in dioxan (4.0 M, 6.0 mL) was added then the reaction was concentrated to dryness. MeOH was added and the mixture filtered. Crystallisation of the product was induced by addition of Et₂O. The product was filtered and dried under high vacuum.

General Procedure 2 (GP2)

The aldehyde or ketone and aminoguanidine hydrochloride (0.95 or 1.0 equivalent) in EtOH (2 mL) were shaken at 70° C. for 18 hours then cooled to room temperature. The reaction was filtered and the precipitate washed with EtOAc (2 times), DCM (2 times), Et₂O (2 times) and dried under high vacuum.

General Procedure 3 (GP3)

The aldehyde or ketone and aminoguanidine hydrochloride (0.95 or 1.0 equivalent) in EtOH (2 mL) were shaken at 70° C. for 18 hours then cooled to room temperature. Et₂O (2-20 mL) was added to induce crystallisation. The reaction was filtered and the precipitate washed with EtOAc (2 times), DCM (2 times), Et₂O (2 times) and dried under high vacuum.

General Procedure 4 (GP4)

The aldehyde or ketone and aminoguanidine hydrochloride (0.95 or 1.0 equivalent) in EtOH (2 mL) were shaken at 70° C. for 18 hours then cooled to room temperature. Et₂O was added but no crystallisation occurred. Water (20 mL) was added and the aqueous layer washed with EtOAc (2×20 mL). NaOH (2M, 5 mL) was added to the aqueous layer and the product was extracted with EtOAc (2×20 mL). The organic layer was dried over MgSO₄, filtered and concentrated.

General Procedure 5 (GP5)

3-Chloro-4-hydroxybenzaldehyde (1.2 mmol, 188 mg) in acetone (1 mL) was added to an alkyl halide (1.0 mmol), potassium carbonate (powder, 1.2 mmol, 166 mg) in acetone (1 mL). The reaction was heated to 40° C. for 72 h then 55° C. for 24 h. The reaction was cooled and filtered through a 45 μm filter, washing with acetone.

General Procedure 6 (GP6)

The aldehyde or ketone and aminoguanidine hydrochloride (0.95 or 1.0 equivalent) in EtOH (2 mL) were shaken at 70° C. for 18 hours then cooled to room temperature. Et₂O (2-20 mL) was added to induce crystallisation but this resulted in oiling. However, addition of a small amount of DCM resulted in crystallisation. The precipitate was filtered and washed with EtOAc (2 times), DCM (2 times), Et₂O (2 times) and dried under high vacuum.

General Procedure 7 (GP7)

The aldehyde and aminoguanidine hydrochloride (1 equivalent) in EtOH (1 mL/mmol) were heated in a microwave at 130° C. for 12 minutes then cooled to room temperature. The reaction was filtered and the precipitate washed with EtOAc (2 times), DCM (2 times), Et₂O (2 times) and dried under high vacuum.

General Procedure 8 (GP8)

The aldehyde and aminoguanidine hydrochloride (1 equivalent) in EtOH (1 mL/mmol) were heated in a microwave at 130° C. for 12 minutes then cooled to room temperature. Et₂O (2-4 mL) was then added to induce crystallization. The reaction was filtered and the precipitate washed with EtOAc (2 times), DCM (2 times), Et₂O (2 times) and dried under high vacuum.

### EXAMPLES

### Example 1

### 1-(4-Fluorobenzylideneamino)guanidine Hydrochloride (2001)

4-Fluorobenzaldehyde (5.0 mmol, 621 mg) was used according to GP1 to give the title compound (2001) as a white powder (534 mg, 49%). ¹H NMR (CD₃OD) δ 8.13 (s, 1H), 7.85 (m, 2H), 7.17 (m, 2H); HPLC-MS (ammonium acetate) [M+H]⁺=181.1.

### Example 2

### 1-[3-(Trifluoromethyl)benzylideneamino]guanidine Hydrochloride (2002)

3-(Trifluoromethyl)benzaldehyde (5.0 mmol, 871 mg) was used according to GP1 to give the title compound (2002) as a white powder (643 mg, 48%). ¹H NMR (CD₃OD) δ 8.22 (s, 1H), 8.17 (m, 1H), 8.05 (m, 1H), 7.74 (m, 1H), 7.65 (m, 1H); HPLC-MS (ammonium acetate) [M+H]⁺=231.1.

### Example 3

### 1-[1-(3-Bromophenyl)ethylideneamino]guanidine Hydrochloride (2003)

3′-Bromoacetophenone (5.0 mmol, 995 mg) was used according to GP1 to give the title compound (2003) as a white powder (977 mg, 67%). ¹H NMR (CD₃OD) δ 8.12 (ap. t, J=1.7 Hz, 1H), 7.84 (ddd, J=8.0, 1.7, 1.0 Hz, 1H), 7.59 (ddd, J=7.8, 2.0, 1.0 Hz, 1H), 7.35 (ap. t, J=8.0 Hz, 1H), 2.36 (s, 3H); HPLC-MS (ammonium acetate) [M+H]⁺=255.1, 257.1

### Example 4

### 1-(5-Fluoro-2-nitrobenzylideneamino)guanidine Hydrochloride (2004)

5-Fluoro-2-nitrobenzaldehyde (5.0 mmol, 846 mg) was used according to GP1 to give the title compound (2004) as a beige powder (989 mg, 76%). ¹H NMR (CD₃OD) δ 8.67 (d, J=1.6 Hz, 1H), 8.21 (dd, J=9.4, 4.9 Hz, 1H), 8.11 (dd, J=9.4, 2.9 Hz, 1H), 7.41 (m, 1H); HPLC-MS (ammonium acetate) [M+H]⁺=226.1.

### Example 5

### 1-[(Benzo[1,3]dioxol-5-yl methylideneamino]guanidine Hydrochloride (2005)

Benzo[1,3]dioxole-5-carbaldehyde (5.0 mmol, 751 mg) was used according to GP1 to give the title compound (2005) as a white powder (737 mg, 61%). ¹H NMR (CD₃OD) δ 7.99 (s, 1H), 7.47 (d, J=1.6 Hz, 1H), 7.15 (dd, J=8.0, 1.6 Hz, 1H), 6.87 (d, J=8.0 Hz, 1H), 6.01 (s, 2H); HPLC-MS (ammonium acetate) [M+H]⁺=207.1.

### Example 6

### 1-[(Anthracen-9-yl)methylideneamino]guanidine Hydrochloride (2006)

9-Anthraldehyde (5.0 mmol, 1.03 g) was used according to GP1 to give the title compound (2006) as a yellow powder (133 mg, 9%). ¹H NMR (CD₃OD) δ 9.26 (s, 1H), 8.65 (s, 1H), 8.48 (m, 2H), 8.11 (m, 2H), 7.61 (m, 2H), 7.55 (m, 2H); HPLC-MS (ammonium acetate) [M+H]⁺=263.2.

### Example 7

### 1-(3,5-Dimethoxybenzylideneamino)guanidine Hydrochloride (2007)

3,5-Dimethoxybenzaldehyde (2.0 mmol, 332 mg) and aminoguanidine hydrochloride (2.0 mmol, 220 mg) were used according to GP3 to give the title compound (2007) as a white powder (516 mg, 99%). ¹H NMR (CD₃OD) δ 8.03 (s, 1H), 6.97 (d, J=2.3 Hz, 2H), 6.57 (t, J=2.3 Hz, 1H), 3.82 (s, 6H); HPLC-MS (ammonium acetate) [M+H]⁺=223.3.

### Example 8

### 1-(2,4-Dichlorobenzylideneamino)guanidine Hydrochloride (2008)

2,4-Dichlorobenzaldehyde (2.0 mmol, 350 mg) and aminoguanidine hydrochloride (2.0 mmol, 220 mg) were used according to GP2 to give the title compound (2008) as a white powder (461 mg, 86%). ¹H NMR (CD₃OD) δ 8.52 (s, 1H), 8.18 (d, J=8.6 Hz, 1H), 7.55 (d, J=2.0 Hz, 1H), 7.41 (m, 1H); HPLC-MS (ammonium acetate) [M+H]⁺=231.2.

### Example 9

### 1-(3-Fluoro-4-methoxybenzylideneamino)guanidine Hydrochloride (2009)

3-Fluoro-4-methoxybenzaldehyde (2.0 mmol, 308 mg) and aminoguanidine hydrochloride (2.0 mmol, 220 mg) were used according to GP2 to give the title compound (2009) as a white powder (449 mg, 91%). ¹H NMR (CD₃OD) δ 8.04 (d, J=1.4 Hz, 1H), 7.71 (m, 1H), 7.45 (m, 1H), 7.14 (t, J=8.4 Hz, 1H), 3.92 (s, 3H); HPLC-MS (ammonium acetate) [M+H]⁺=211.2.

### Example 10

### 1-(3-Bromo-4-fluorobenzylideneamino)guanidine Hydrochloride (2010)

3-Bromo-4-fluorobenzaldehyde (2.0 mmol, 406 mg) and aminoguanidine hydrochloride (2.0 mmol, 220 mg) were used according to GP2 to give the title compound (2010) as a white powder (445 mg, 75%). ¹H NMR (CD₃OD) δ 8.20 (dd, J=6.8, 2.2 Hz, 1H), 8.08 (s, 1H), 7.79 (ddd, J=8.6, 4.7, 2.2 Hz, 1H), 7.29 (t, J=8.6 Hz, 1H); HPLC-MS (ammonium acetate) [M+H]⁺=259.2, 261.2.

### Example 11

### 1-(3,4,5-Trimethoxybenzylideneamino)guanidine Hydrochloride (2011)

3,4,5-Trimethoxybenzaldehyde (2.0 mmol, 392 mg) and aminoguanidine hydrochloride (2.0 mmol, 220 mg) were used according to GP3 to give the title compound (2011) as a white powder 565 (mg, 97%). ¹H NMR (CD₃OD) δ 8.05 (s, 1H), 7.14 (s, 2H), 3.89 (s, 6H), 3.80 (s, 3H); HPLC-MS (ammonium acetate) [M+H]⁺=253.3.

### Example 12

### 1-(4-Fluoro-3-methylbenzylideneamino)guanidine Hydrochloride (2012)

4-Fluoro-3-methylbenzaldehyde (2.0 mmol, 276 mg) and aminoguanidine hydrochloride (2.0 mmol, 220 mg) were used according to GP3 to give the title compound (2012) as a white powder (433 mg, 93%). ¹H NMR (CD₃OD) δ 8.05 (s, 1H), 7.75 (m, 1H), 7.63 (m, 1H), 7.10 (t, J=9.2 Hz, 1H), 2.31 (d, J=2.0 Hz, 3H); HPLC-MS (ammonium acetate) [M+H]⁺=295.2

### Example 13

### 1-(3-Chloro-4-fluorobenzylideneamino)guanidine Hydrochloride (2013)

3-Chloro-4-fluorobenzaldehyde (2.0 mmol, 317 mg) and aminoguanidine hydrochloride (2.0 mmol, 220 mg) were used according to GP2 to give the title compound (2013) as a white powder (391 mg, 77%). ¹H NMR (CD₃OD) δ 8.08 (s, 1H), 8.06 (dd, J=7.2, 2.2 Hz, 1H), 7.74 (ddd, J=8.6, 4.7, 2.2 Hz, 1H), 7.32 (ap. t, J=8.8 Hz, 1H); HPLC-MS (ammonium acetate) [M+H]⁺=215.2, 217.2.

### Example 14

### 1-(3-Bromo-4-methoxybenzylideneamino)guanidine Hydrochloride (2014)

3-Bromo-4-methoxybenzaldehyde (2.0 mmol, 430 mg) and aminoguanidine hydrochloride (2.0 mmol, 220 mg) were used according to GP2 to give the title compound (2014) as a pale yellow powder (568 mg, 92%). ¹H NMR (CD₃OD) δ 8.12 (d, J=2.2 Hz, 1H), 8.01 (s, 1H), 8.11 (dd, J=8.6, 2.2 Hz, 1H), 7.69 (d, J=8.6 Hz, 1H), 3.93 (s, 3H); HPLC-MS (ammonium acetate) [M+H]⁺=271.2, 273.2.

### Example 15

### 1-(2,5-Difluorobenzylideneamino)guanidine Hydrochloride (2015)

2,5-Difluorobenzaldehyde (2.0 mmol, 284 mg) and aminoguanidine hydrochloride (2.0 mmol, 220 mg) were used according to GP2 to give the title compound (2015) as a white powder (377 mg, 80%). ¹H NMR (CD₃OD) δ 8.31 (d, J=2.0 Hz, 1H), 7.92 (m, 1H), 7.23 (m, 2H); HPLC-MS (ammonium acetate) [M+H]⁺=199.2.

### Example 16

### 1-(2,4-Difluorobenzylideneamino)guanidine Hydrochloride (2016)

2,4-Difluorobenzaldehyde (2.0 mmol, 284 mg) and aminoguanidine hydrochloride (2.0 mmol, 220 mg) were used according to GP3 to give the title compound (2016) as a white powder (418 mg, 89%). ¹H NMR (CD₃OD) δ 8.30 (s, 1H), 8.16 (m, 1H), 7.07 (m, 2H); HPLC-MS (ammonium acetate) [M+H]⁺=199.2.

### Example 17

### 1-(2,3-Dichlorobenzylideneamino)guanidine Hydrochloride (2017)

2,3-Dichlorobenzaldehyde (2.0 mmol, 350 mg) and aminoguanidine hydrochloride (2.0 mmol, 220 mg) were used according to GP2 to give the title compound (2017) as a white powder (441 mg, 82%). ¹H NMR (CD₃OD) δ 8.60 (s, 1H), 8.13 (dd, J=8.0, 1.6 Hz, 1H), 7.63 (dd, J=8.0, 1.6 Hz, 1H), 7.37 (m, 1H); HPLC-MS (ammonium acetate) [M+H]⁺=231.2, 233.2, 235.2.

### Example 18

### 1-(4-Bromo-2-fluorobenzylideneamino)guanidine Hydrochloride (2018)

4-Bromo-2-fluorobenzaldehyde (2.0 mmol, 406 mg) and aminoguanidine hydrochloride (2.0 mmol, 220 mg) were used according to GP2 to give the title compound (2018) as a white powder (441 mg, 74%). ¹H NMR (CD₃OD) δ 8.30 (s, 1H), 8.04 (m, 1H), 7.46 (m, 2H); HPLC-MS (ammonium acetate) [M+H]⁺=259.2, 261.2.

### Example 19

### 1-(4-Phenylbenzylideneamino)guanidine Hydrochloride (2019)

4-Biphenylcarboxaldehyde (2.0 mmol, 364 mg) and aminoguanidine hydrochloride (2.0 mmol, 220 mg) were used according to GP2 to give the title compound (2019) as a white powder (440 mg, 80%). ¹H NMR (CD₃OD) δ 8.15 (s, 1H), 7.88 (m, 2H), 7.71 (m, 2H), 7.66 (m, 2H), 7.46 (m, 2H), 7.38 (m, 1H); HPLC-MS (ammonium acetate) [M+H]⁺=239.3.

### Example 20

### 1-(4-Phenoxybenzylideneamino)guanidine Hydrochloride (2020)

4-Phenoxybenzaldehyde (2.0 mmol, 396 mg) and aminoguanidine hydrochloride (2.0 mmol, 220 mg) were used according to GP4 to give the title compound (2020) as a pale pink powder (384 mg, 75%). ¹H NMR (CD₃OD) δ 8.01 (s, 1H), 7.67 (m, 2H), 7.36 (m, 2H), 7.13 (m, 1H), 7.01 (m, 2H), 6.96 (m, 2H); HPLC-MS (ammonium acetate) [M+H]⁺=255.3.

### Example 21

### 1-(3-Phenoxybenzylideneamino)guanidine Hydrochloride (2021)

3-Phenoxybenzaldehyde (2.0 mmol, 396 mg) and aminoguanidine hydrochloride (2.0 mmol, 220 mg) were used according to GP4 to give the title compound (2021) as a pale pink powder (301 mg, 59%). ¹H NMR (CD₃OD) δ 7.99 (s, 1H); 7.30-7.43 (m, 4H), 7.10 (m, 1H), 7.00 (m, 2H), 6.90 (m, 1H); HPLC-MS (ammonium acetate) [M+H]⁺=255.3.

### Example 22

### 1-(3,5-Di-tert-butyl-2-hydroxybenzylideneamino)guanidine (2022)

3,5-Di-tert-butyl-2-hydroxybenzaldehyde (2.0 mmol, 469 mg) and aminoguanidine hydrochloride (2.0 mmol, 220 mg) were used according to GP4 to give the title compound (2022) as a pale yellow/brown powder (501 mg, 86%). ¹H NMR (CD₃OD) δ 8.19 (s, 1H), 7.28 (d, J=2.5 Hz, 1H), 7.07 (d, J=2.5 Hz, 1H), 1.44 (s, 9H), 1.30 (s, 9H); HPLC-MS (ammonium acetate) [M+H]⁺=.

### Example 23

### 1-(2,3,5-Trichlorobenzylideneamino)guanidine Hydrochloride (2023)

2,3,5-Trichlorobenzaldehyde (2.0 mmol, 419 mg) and aminoguanidine hydrochloride (2.0 mmol, 220 mg) were used according to GP2 to give the title compound (2023) as a white powder (410 mg, 68%). ¹H NMR (CD₃OD) δ 8.54 (s, 1H), 8.26 (d, J=2.4 Hz, 1H), 7.72 (d, J=2.4 Hz, 1H); HPLC-MS (ammonium acetate) [M+H]⁺=265.1, 267.1, 279.1.

### Example 24

### 1-(3,5-Dibromo-4-hydroxybenzylideneamino)guanidine Hydrochloride (2024)

3,5-Dibromo-4-hydroxybenzaldehyde (2.0 mmol, 560 mg) and aminoguanidine hydrochloride (2.0 mmol, 220 mg) were used according to GP2 to give the title compound (2024) as a yellow powder (701 mg, 94%). ¹H NMR (CD₃OD) δ 7.98 (s, 2H), 7.96 (s, 1H); HPLC-MS (ammonium acetate) [M+H]⁺=3335.1, 337.1, 339.1.

### Example 25

### 1-(4-Isopropoxybenzylideneamino)guanidine (2025)

4-Isopropoxybenzaldehyde (2.0 mmol, 328 mg) and aminoguanidine hydrochloride (2.0 mmol, 220 mg) were used according to GP4 to give the title compound (2025) as a cream powder (295 mg, 67%). ¹H NMR (CD₃OD) δ 7.98 (s, 1H), 7.59 (m, 2H), 6.88 (m, 2H), 4.62 (sept, J=6.0 Hz, 1H), 1.31 (d, J=6.0 Hz, 6H); HPLC-MS (ammonium acetate) [M+H]⁺=221.2.

### Example 26

### 1-(3,4-Diethoxybenzylideneamino)guanidine (2026)

3,4-Diethoxybenzaldehyde (2.0 mmol, 388 mg) and aminoguanidine hydrochloride (2.0 mmol, 220 mg) were used according to GP4 to give the title compound (2026) as a white powder (355 mg, 71%). ¹H NMR (CD₃OD) δ 7.95 (s, 1H), 7.39 (d, J=2.0 Hz, 1H), 7.11 (dd, J=8.2, 2.0 Hz, 1H), 6.91 (d, J=8.0 Hz, 1H), 4.11 (q, J=7.0 Hz, 2H), 4.09 (q, J=7.0 Hz, 2H), 1.42 (t, J=7.0 Hz, 3H), 1.41 (t, J=7.0 Hz, 3H); HPLC-MS (ammonium acetate) [M+H]⁺=251.1.

### Example 27

### 1-(3,5-Difluorobenzylideneamino)guanidine Hydrochloride (2027)

3,5-Difluorobenzaldehyde (2.0 mmol, 284 mg) and aminoguanidine hydrochloride (2.0 mmol, 220 mg) were used according to GP2 to give the title compound (2027) as white crystals (412 mg, 87%). ¹H NMR (CD₃OD) δ 8.12 (s, 1H), 7.47 (m, 2H), 7.03 (tt, J=9.0, 2.4 Hz, 1H); HPLC-MS (ammonium acetate) [M+H]⁺=199.1.

### Example 28

### 1-(3,4-Dibromobenzylideneamino)guanidine Hydrochloride (2028)

Fluorene-2-carboxaldehyde (2.0 mmol, 388 mg) and aminoguanidine hydrochloride (2.0 mmol, 220 mg) were used according to GP2 to give the title compound (2028) as a pale yellow powder (559 mg, 97%). ¹H NMR (CD₃OD) δ 8.16 (s, 1H), 8.01 (br. s, 1H), 7.85 (m, 2H), 7.77 (m, 1H), 7.57 (m, 1H), 7.38 (m, 1H), 7.34 (dt, J=7.4, 1.2 Hz, 1H), 3.93 (s, 2H); HPLC-MS (ammonium acetate) [M+H]⁺=251.1.

### Example 29

### 1-(3,4-Dibromobenzylideneamino)guanidine Hydrochloride (3093)

3,4-Dibromobenzaldehyde (2.0 mmol, 528 mg) and aminoguanidine hydrochloride (2.0 mmol, 220 mg) were used according to GP2 to give the title compound (3093) as a white powder (655 mg, 92%). ¹H NMR (CD₃OD) δ 8.20 (d, J=2.0 Hz, 1H), 8.06 (s, 1H), 7.75 (d, J=8.4 Hz, 1H), 7.65 (dd, J=8.4, 2.0 Hz, 1H); HPLC-MS (ammonium acetate) [M+H]⁺=318.8, 320.8, 322.8.

### Example 30

### 1-(4-Chloro-3-fluorobenzylideneamino)guanidine Hydrochloride (2030)

4-Chloro-3-fluorobenzaldehyde (2.0 mmol, 317 mg) and aminoguanidine hydrochloride (2.0 mmol, 220 mg) were used according to GP2 to give the title compound (2030) as white crystals (466 mg, 93%). ¹H NMR (CD₃OD) δ 8.12 (s, 1H), 7.81 (m, 1H), 7.56 (m, 2H); HPLC-MS (ammonium acetate) [M+H]⁺=215.0, 217.0.

### Example 31

### 1-(3-Chloro-4-hydroxybenzylideneamino)guanidine Hydrochloride (2031)

3-Chloro-4-hydroxybenzaldehyde (2.0 mmol, 313 mg) and aminoguanidine hydrochloride (2.0 mmol, 220 mg) were used according to GP2 to give the title compound (2031) as a yellow powder (468 mg, 94%). ¹H NMR (CD₃OD) δ 7.97 (s, 1H), 7.84 (d, J=2.1 Hz, 1H), 7.52 (dd, J=8.4, 2.1 Hz, 1H), 6.94 (d, J=8.4 Hz, 1H); HPLC-MS (ammonium acetate) [M+H]⁺=213.1, 215.0.

### Example 32

### 1-(4-Fluoro-3-nitrobenzylideneamino)guanidine Hydrochloride (2032)

2-Fluoro-5-formylbenzonitrile (2.0 mmol, 298 mg) and aminoguanidine hydrochloride (2.0 mmol, 220 mg) were used according to GP2 to give the title compound (2032) as white crystals (452 mg, 93%). ¹H NMR (CD₃OD) δ 8.32 (dd, J=6.2, 2.2 Hz, 1H), 8.15 (s, 1H), 8.14 (ddd, J=8.8, 5.2, 2.2 Hz, 1H), 7.45 (t, J=8.8 Hz, 1H); HPLC-MS (ammonium acetate) [M+H]⁺=206.1.

### Example 33

### 1-(3,5-Dimethyl-4-hydroxybenzylideneamino)guanidine Hydrochloride (2033)

3,5-Dimethyl-4-hydroxybenzaldehyde (2.0 mmol, 300 mg) and aminoguanidine hydrochloride (2.0 mmol, 220 mg) were used according to GP2 to give the title compound (2033) as a yellow powder (462 mg, 95%). ¹H NMR (CD₃OD) δ 7.94 (s, 1H), 7.39 (s, 2H), 2.24 (s, 6H); HPLC-MS (ammonium acetate) [M+H]⁺=207.1.

### Example 34

### 1-(4-Methoxy-2,3-dimethylbenzylideneamino)guanidine Hydrochloride (2034)

4-Methoxy-2,3-dimethylbenzaldehyde (2.0 mmol, 328 mg) and aminoguanidine hydrochloride (2.0 mmol, 220 mg) were used according to GP2 to give the title compound (2034) as a yellow powder (461 mg, 89%). ¹H NMR (CD₃OD) δ 8.45 (s, 1H), 7.83 (d, J=8.8 Hz, 1H), 6.86 (d, J=8.8 Hz, 1H), 3.85 (s, 3H), 2.37 (s, 3H), 2.17 (s, 3H); HPLC-MS (ammonium acetate) [M+H]⁺=221.1.

### Example 35

### 1-[4-Chloro-3-(trifluoromethyl)benzylideneamino]guanidine Hydrochloride (2035)

4-Chloro-3-(trifluoromethyl)benzaldehyde (2.0 mmol, 417 mg) and aminoguanidine hydrochloride (2.0 mmol, 220 mg) were used according to GP2 to give the title compound (2035) as a white powder (524 mg, 87%). ¹H NMR (CD₃OD) δ 8.24 (d, J=2.0 Hz, 1H), 8.18 (s, 1H), 8.04 (dd, J=8.4, 2.0 Hz, 1H), 7.69 (d, J=8.4 Hz, 1H); HPLC-MS (ammonium acetate) [M+H]⁺=265.0, 267.0.

### Example 36

### 1-(3-Bromo-4,5-dimethoxybenzylideneamino)guanidine Hydrochloride (3099)

3-Bromo-4,5-dimethoxybenzaldehyde (2.0 mmol, 490 mg) and aminoguanidine hydrochloride (2.0 mmol, 220 mg) were used according to GP3 to give the title compound (3099) as a white powder (588 mg, 87%). ¹H NMR (CD₃OD) δ 8.02 (s, 1H), 7.56 (d, J=1.9 Hz, 1H), 7.52 (d, J=1.9 Hz, 1H), 3.94 (s, 3H), 3.85 (s, 3H); HPLC-MS (ammonium acetate) [M+H]⁺=300.9, 302.9.

### Example 37

### 1-[3,4-Dihydro-2H-benzo[b][1,4]dioxepin-7-yl)methylideneamino]guanidine Hydrochloride (2038)

3,4-Dihydro-2H-benzo[b][1,4]dioxepine-7-carbaldehyde (1.0 mmol, 178 mg) and aminoguanidine hydrochloride (1.0 mmol, 110 mg) were used according to GP3 to give the title compound (2038) as a white powder (206 mg, 76%). ¹H NMR (CD₃OD) δ 8.00 (s, 1H), 7.43 (d, J=2.2 Hz, 1H), 7.35 (dd, J=8.4, 2.2 Hz, 1H), 6.99 (d, J=8.4 Hz, 1H), 4.23 (t, J=5.6 Hz, 2H), 4.21 (t, J=5.6 Hz, 2H), 2.19 (pent, J=5.6 Hz, 2H); HPLC-MS (ammonium acetate) [M+H]⁺=235.1.

### Example 38

### [(Cyclohexylphenylmethylideneamino]guanidine (2039)

Benzoylcyclohexane (2.0 mmol, 377 mg) and aminoguanidine hydrochloride (2.0 mmol, 220 mg) were used according to GP4. The crude material was purified on the CombiFlash using method CF1 to give the title compound (2039) as a cream powder (109 mg, 22%). ¹H NMR (CD₃OD) δ 7.42 (m, 2H), 7.35 (m, 1H), 7.18 (m, 2H), 2.48 (m, 1H), 1.85 (m, 2H), 1.77 (m, 2H), 1.67 (m, 1H), 1.14-1.39 (m, 5H); HPLC-MS (ammonium acetate) [M+H]⁺=245.2.

### Example 39

### 1-[1-(2,3-Dihydro-benzo[1,4]dioxin-6-yl)ethylideneamino]guanidine Hydrochloride (2040)

1-(2,3-Dihydro-benzo[1,4]dioxin-6-yl)ethanone (2.0 mmol, 356 mg) and aminoguanidine hydrochloride (2.0 mmol, 220 mg) were used according to GP3 to give the title compound (2040) as a yellow powder (492 mg, 91%). ¹H NMR (CD₃OD) δ 7.42 (d, J=2.2 Hz, 1H), 7.37 (dd, J=8.6, 2.2 Hz, 1H), 6.86 (d, J=8.6 Hz, 1H), 4.27 (m, 4H), 2.30 (s, 3H); HPLC-MS (ammonium acetate) [M+H]⁺=235.1.

### Example 40

### 1-(4-Benzyloxy-3-chlorobenzylideneamino)guanidine Hydrochloride (2041)

Benzyl bromide (1.0 mmol, 171 mg) was used according to GP5 and the crude material was purified using CF2 to give 4-benzyloxy-3-chlorobenzaldehyde as a white powder (224 mg, 91%). ¹H NMR (CDCl₃) δ 9.86 (s, 1H), 7.93 (d, J=2.0 Hz, 1H), 7.73 (dd, J=8.4, 2.0 Hz, 1H), 7.32-7.48 (m, 5H), 7.08 (d, J=8.4 Hz, 1H), 5.26 (s, 2H).

4-Benzyloxy-3-chlorobenzaldehyde (0.91 mmol, 224 mg) and aminoguanidine hydrochloride (0.86 mmol, 95 mg) were used according to GP3 to give the title compound (2041) as a white powder (238 mg, 77%). ¹H NMR (CD₃OD) δ 8.01 (s, 1H), 7.98 (d, J=2.2 Hz, 1H), 7.61 (dd, J=8.6, 2.2 Hz, 1H), 7.47 (m, 2H), 7.37 (m, 2H), 7.34 (m, 1H), 7.19 (d, J=8.6 Hz, 1H), 5.24 (s, 2H); HPLC-MS (ammonium acetate) [M+H]⁺=303.0, 305.0.

### Example 41

### 1-(4-Allyloxy-3-chlorobenzylideneamino)guanidine Hydrochloride (2042)

Allyl bromide (1.0 mmol, 121 mg) was used according to GP5 and the crude material was purified using CF2 to give 4-allyloxy-3-chlorobenzaldehyde as pale yellow crystals (181 mg, 92%). ¹H NMR (CDCl₃) δ 9.85 (s, 1H), 7.91 (d, J=2.0 Hz, 1H), 7.74 (dd, J=8.4, 2.0 Hz, 1H), 7.02 (d, J=8.4 Hz, 1H), 6.06 (ddt, J=17.2, 10.6, 5.1 Hz, 1H), 5.49 (m, 1H), 5.36 (m, 1H), 4.71 (dt, J=5.1, 1.7 Hz, 2H).

4-Allyloxy-3-chlorobenzaldehyde (0.92 mmol, 181 mg) and aminoguanidine hydrochloride (0.87 mmol, 96 mg) were used according to GP3 to give the title compound (2042) as a white powder (189 mg, 71%). ¹H NMR (CD₃OD) δ 7.97 (s, 1H), 7.96 (d, J=2.0 Hz, 1H), 7.62 (dd, J=8.6, 2.0 Hz, 1H), 7.12 (d, J=8.6 Hz, 1H), 6.08 (ddt, J=17.4, 10.6, 5.1 Hz, 1H), 5.47 (ap. dq, 17.4, 1.6 Hz, 1H), 5.32 (ap. dq, J=10.6, 1.6 Hz, 1H), 4.70 (dt, J=5.1, 1.6 Hz, 2H); HPLC-MS (ammonium acetate) [M+H]⁺=304.9, 306.9.

### Example 42

### 1-(3-Chloro-4-methoxybenzylideneamino)guanidine Hydrochloride (2043)

Iodomethane (1.0 mmol, 142 mg) was used according to GP5 and the crude material was purified using CF2 to give 3-chloro-4-methoxybenzaldehyde as a white solid (182 mg, 100%). ¹H NMR (CDCl₃) δ 9.85 (s, 1H), 7.90 (d, J=2.0 Hz, 1H), 7.77 (dd, J=8.4, 2.0 Hz, 1H), 7.04 (d, J=8.4 Hz, 1H), 3.99 (s, 3H).

3-Chloro-4-methoxybenzaldehyde (1.0 mmol, 182 mg) and aminoguanidine hydrochloride (0.95 mmol, 104 mg) were used according to GP2 to give the title compound (2043) as a white powder (219 mg, 83%). ¹H NMR (CD₃OD) δ 8.01 (s, 1H), 7.95 (d, J=2.2 Hz, 1H), 7.64 (dd, J=8.6, 2.2 Hz, 1H), 7.13 (d, J=8.6 Hz, 1H), 3.94 (s, 3H); HPLC-MS (ammonium acetate) [M+H]⁺=227.1, 229.0.

### Example 43

### 1-[3-Chloro-4-(4-cyanobutoxy)benzylideneamino]guanidine Hydrochloride (2044)

5-Bromopentanenitrile (1.0 mmol, 162 mg) was used according to GP5 and the crude material was purified using CF3 to give 5-(2-chloro-4-formylphenoxy)-pentanenitrile as a colourless oil (151 mg, 63%). ¹H NMR (CDCl₃) δ 9.85 (s, 1H), 7.91 (d, J=2.0 Hz, 1H), 7.76 (dd, J=8.4, 2.0 Hz, 1H), 7.01 (d, J=8.4 Hz, 1H), 4.17 (t, J=5.8 Hz, 2H), 2.52 (t, J=7.0 Hz, 2H), 2.07 (m, 2H), 1.95 (m, 2H).

5-(2-Chloro-4-formylphenoxy)-pentanenitrile (0.63 mmol, 151 mg) and aminoguanidine hydrochloride (0.60 mmol, 66 mg) were used according to GP3 to give the title compound (2044) as a pale yellow powder (161 mg, 77%). ¹H NMR (CD₃OD) δ 8.01 (s, 1H), 7.97 (d, J=2.2 Hz, 1H), 7.63 (dd, J=8.8, 2.2 Hz, 1H), 7.13 (d, J=8.8 Hz, 1H), 4.18 (t, J=5.9 Hz, 2H), 2.59 (t, J=7.0 Hz, 2H), 2.01 (m, 2H), 1.90 (m, 2H); HPLC-MS (ammonium acetate) [M+H]⁺=294.0, 296.0

### Example 44

### 1-[3-Chloro-4-(3-phenoxypropoxy)benzylideneamino]guanidine Hydrochloride (2045)

3-(Bromopropoxy)benzene (1.0 mmol, 215 mg) was used according to GP5 and the crude material was purified using CF4 to give 3-chloro-4-(3-phenoxypropoxy)benzaldehyde as a white powder (140 mg, 48%). ¹H NMR (CDCl₃) δ 9.85 (s, 1H), 7.90 (d, J=2.0 Hz, 1H), 7.75 (dd, J=8.6, 2.0 Hz, 1H), 7.28 (m, 2H), 7.06 (d, J=8.6 Hz, 1H), 6.93 (m, 3H), 4.34 (t, J=6.0 Hz, 2H), 4.22 (t, J=6.0 Hz, 2H), 2.30 (pent, J=6.0 Hz, 2H).

3-Chloro-4-(3-phenoxypropoxy)benzaldehyde (0.48 mmol, 140 mg) and aminoguanidine hydrochloride (0.46 mmol, 50 mg) were used according to GP3 to give the title compound (2045) as a white powder (159 mg, 86%). ¹H NMR (CD₃OD) δ 8.01 (s, 1H), 7.95 (d, J=2.2 Hz, 1H), 7.62 (dd, J=8.6, 2.2 Hz, 1H), 7.25 (m, 2H), 7.15 (d, J=8.6 Hz, 1H), 6.92 (m, 3H), 4.31 (t, J=6.0 Hz, 2H), 4.21 (t, J=6.0 Hz, 2H), 2.30 (pent, J=6.0 Hz, 2H); HPLC-MS (ammonium acetate) [M+H]⁺=347.0, 349.0

### Example 45

### 1-[3-Chloro-4-(2-phenylethoxy)benzylideneamino]guanidine Hydrochloride (2046)

2-Bromoethyl benzene (1.0 mmol, 185 mg) was used according to GP5 and the crude material was purified using CF4 to give 3-chloro-4-(2-phenylethoxy)benzaldehyde as a colourless oil (146 mg, 56%). ¹H NMR (CDCl₃) δ 9.83 (s, 1H), 7.89 (d, J=2.0 Hz, 1H), 7.72 (dd, J=8.4, 2.0 Hz, 1H), 7.33 (m, 4H), 7.27 (m, 1H), 6.98 (d, J=8.4 Hz, 1H), 4.30 (t, J=6.9 Hz, 2H), 3.20 (t, J=6.9 Hz, 2H).

3-Chloro-4-(2-phenylethoxy)benzaldehyde (0.56 mmol, 146 mg) and aminoguanidine hydrochloride (0.55 mmol, 58 mg) in EtOH (2 mL) were shaken at 70° C. for 18 hours then cooled to room temperature. Et₂O was added to induce. crystallisation and the precipitate, which was starting aminoguanidine hydrochloride, was filtered off and discarded. A new precipitate was present in the filtrate, so the filtrate was filtered and washed with 1:1 DCM:EtOAc (2 times), Et₂O (2 times) and dried under high vacuum to give the title compound (2046) as a white powder (70 mg, 35%). ¹H NMR (CD₃OD) δ 8.00 (s, 1H), 7.95 (d, J=2.2 Hz, 1H), 7.60 (dd, J=8.6, 2.2 Hz, 1H), 7.34 (m, 2H), 7.29 (m, 2H), 7.22 (m, 1H), 7.10 (d, J=8.6 Hz, 1H), 4.30 (t, J=6.7 Hz, 2H), 3.13 (t, J=6.7 Hz, 2H); HPLC-MS (ammonium acetate) [M+H]⁺=317.0, 319.0.

### Example 46

### 1-(3-Chloro-4-hexyloxybenzylideneamino)guanidine Hydrochloride (2047)

1-Iodohexane (1.0 mmol, 212 mg) was used according to GP5 and the crude material was purified using CF5 to give 3-chloro-4-hexyloxybenzaldehyde as a white solid (208 mg, 86%). ¹H NMR (CDCl₃) δ 9.85 (s, 1H), 7.90 (d, J=2.0 Hz, 1H), 7.74 (dd, J=8.4, 2.0 Hz, 1H), 7.01 (d, J=8.4 Hz, 1H), 4.12 (t, J=6.5 Hz, 2H), 1.87 (m, 2H), 1.51 (m, 2H), 1.37 (m, 4H), 0.91 (m, 3H).

3-Chloro-4-hexyloxybenzaldehyde (0.86 mmol, 208 mg) and aminoguanidine hydrochloride (0.82 mmol, 90 mg) were used according to GP3 to give the title compound (2047) as a white powder (141 mg, 49%). ¹H NMR (CD₃OD) δ 8.01 (s, 1H), 7.95 (d, J=2.0 Hz, 1H), 7.62 (dd, J=8.6, 2.0 Hz, 1H), 7.10 (d, J=8.6 Hz, 1H), 4.11 (t, J=6.5 Hz, 2H), 1.83 (m, 2H), 1.53 (m, 2H), 1.39 (m, 4H), 0.93 (m, 3H); HPLC-MS (ammonium acetate) [M+H]⁺=297.1, 299.1.

### Example 47

### 1-(3-Chloro-4-propoxyobenzylideneamino)guanidine Hydrochloride (2048)

1-Iodopropane (1.0 mmol, 170 mg) was used according to GP5 and the crude material was purified using CF5 to give 3-chloro-4-propoxybenzaldehyde as a white solid (211 mg, 100%). ¹H NMR (CDCl₃) δ 9.84 (s, 1H), 7.90 (d, J=2.0 Hz, 1H), 7.74 (dd, J=8.4, 2.0 Hz, 1H), 7.01 (d, J=8.4 Hz, 1H), 4.09 (t, J=6.5 Hz, 2H), 1.91 (m, 2H), 1.09 (t, J=7.4 Hz, 3H).

3-Chloro-4-propoxybenzaldehyde (1.0 mmol, 211 mg) and aminoguanidine hydrochloride (0.95 mmol, 104 mg) in EtOH (2 mL) were shaken at 70° C. for 18 hours then cooled to room temperature. Et₂O was added to induce crystallisation and the precipitate, which was starting aminoguanidine hydrochloride, was filtered off and discarded. A new precipitate was present in the filtrate, so the filtrate was filtered and washed with EtOAc (2 times), Et₂O (2 times) and dried under high vacuum to give the title compound (2048) as a white powder (70 mg, 24%). ¹H NMR (CD₃OD) δ 8.00 (s, 1H), 7.95 (d, J=2.2 Hz, 1H), 7.61 (dd, J=8.6, 2.2 Hz, 1H), 7.10 (d, J=8.6 Hz, 1H), 4.07 (t, J=6.3 Hz, 2H), 1.85 (m, 2H), 1.09 (t, J=7.4 Hz, 3H); HPLC-MS (ammonium acetate) [M+H]⁺=297.1, 299.1.

### Example 48

### 1-[3-Chloro-4-(2-methylpropoxy)benzylideneamino]guanidine Acetate (2049)

1-Bromo-2-methylpropane (1.0 mmol, 137 mg) was used according to GP5 and the crude material was purified using CF5 to give 3-chloro-4-(2-methylpropoxy)benzaldehyde as a colourless oil (5 mg, 2%). ¹H NMR (CDCl₃) δ 9.84 (s, 1H), 7.90 (d, J=2.0 Hz, 1H), 7.74 (dd, J=8.4, 2.0 Hz, 1H), 7.00 (d, J=8.4 Hz, 1H), 3.88 (d, J=6.4 Hz, 2H), 2.20 (m, 1H), 1.09 (d, J=6.8 Hz, 6H).

3-Chloro-4-(2-methylpropoxy)benzaldehyde (0.02 mmol, 5 mg) and aminoguanidine hydrochloride (0.04 mmol, 4 mg) in EtOH (1 mL) were shaken at 70° C. for 18 hours then concentrated in vacuo. The crude material was dissolved in CH₃CH:H₂O (3:7, 300 μL) and purified by preparative LC/MS. The fractions containing the desired compound were concentrated in vacuo to give the title compound (2049) as a white powder (6 mg, 94%). ¹H NMR (CD₃OD) δ 8.02 (s, 1H), 7.94 (d, J=2.2 Hz, 1H), 7.60 (dd, J=8.6, 2.2 Hz, 1H), 7.09 (d, J=8.6 Hz, 1H), 3.88 (d, J=6.5 Hz, 2H), 2.13 (m, 1H), 1.94 (s, 3H), 1.08 (d, J=6.7 Hz, 6H); HPLC-MS (ammonium acetate) [M+H]⁺=269.1, 271.1.

### Example 49

### 1-[3-Chloro-4-(4-methylpentoxy)benzylideneamino]guanidine Hydrochloride (2050)

1-Bromo-4-methylpentane (1.0 mmol, 165 mg) was used according to GP5 and the crude material was purified using CF4 to give 3-chloro-4-(4-methylpentoxy)benzaldehyde as a white solid (162 mg, 67%). ¹H NMR (CDCl₃) δ 9.84 (s, 1H), 7.90 (d, J=2.0 Hz, 1H), 7.74 (dd, J=8.4, 2.0 Hz, 1H), 7.01 (d, J=8.4 Hz, 1H), 4.10 (d, J=6.6 Hz, 2H), 1.87 (m, 2H), 1.63 (m, 1H), 1.38 (m, 2H), 0.93 (d, J=6.7 Hz, 6H).

3-Chloro-4-(4-methylpentoxy)benzaldehyde (0.67 mmol, 162 mg) and aminoguanidine hydrochloride (0.64 mmol, 70 mg) were used according to GP3 (but without a DCM wash of the precipitate) to give the title compound (2050) as a white powder (132 mg, 58%). ¹H NMR (CD₃OD) δ 8.00 (s, 1H), 7.95 (d, J=2.2 Hz, 1H), 7.62 (dd, J=8.6, 2.2 Hz, 1H), 7.10 (d, J=8.6 Hz, 1H), 4.10 (d, J=6.4 Hz, 2H), 1.84 (m, 2H), 1.64 (m, 1H), 1.42 (m, 2H), 0.95 (d, J=6.7 Hz, 6H); HPLC-MS (ammonium acetate) [M+H]⁺=297.1, 299.1.

### Example 50

### 1-[3-Chloro-4-(4-cyclohexylmethoxy)benzylideneamino])guanidine Acetate (2051)

Bromomethylcyclohexane (1.0 mmol, 177 mg) was used according to GP5 and the crude material was purified using CF5 to give 3-chloro-4-(4-cyclohexylmethoxy)benzaldehyde as a white solid (6 mg, 2%). ¹H NMR (CDCl₃) δ 9.84 (s, 1H), 7.90 (d, J=2.0 Hz, 1H), 7.74 (dd, J=8.4, 2.0 Hz, 1H), 7.01 (d, J=8.4 Hz, 1H), 3.91 (d, J=5.9 Hz, 2H), 1.84-1.95 (m, 3H), 1.68-1.82 (m, 3H), 1.21-1.39 (m, 3H), 1.05-1.20 (m, 2H).

3-Chloro-4-(4-cyclohexylmethoxy)benzaldehyde (0.02 mmol, 6 mg) and aminoguanidine hydrochloride (0.04 mmol, 4 mg) in EtOH (1 mL) were shaken at 70° C. for 18 hours then concentrated in vacuo. The crude material was dissolved in CH₃CH:H₂O (3:7, 300 μL) and purified by preparative LC/MS. The fractions containing the desired compound were concentrated in vacuo to give the title compound (2051) as a colourless oil (3 mg, 40%). ¹H NMR (CD₃OD) δ 8.01 (s, 1H), 7.93 (d, J=2.2 Hz, 1H), 7.59 (dd, J=8.6, 2.2 Hz, 1H), 7.08 (d, J=8.6 Hz, 1H), 3.91 (d, J=5.9 Hz, 2H), 1.94 (s, 3H), 1.55-1.95 (m, 6H), 1.23-1.41 (m, 3H), 1.09-1.22 (m, 2H); HPLC-MS (ammonium acetate) [M+H]⁺=309.1, 311.1.

### Example 51

### 1-[3-Chloro-4-(2-ethylbutoxy)benzylideneamino]guanidine Acetate (2052)

1-Bromo-2-ethylbutane (1.0 mmol, 165 mg) was used according to GP5 and the crude material was purified using CF5 to give 3-chloro-4-(2-ethylbutoxy)benzaldehyde as a colourless oil (13 mg, 5%). ¹H NMR (CDCl₃) δ 9.84 (s, 1H), 7.90 (d, J=2.0 Hz, 1H), 7.74 (dd, J=8.4, 2.0 Hz, 1H), 7.02 (d, J=8.4 Hz, 1H), 4.01 (d, J=5.7 Hz, 2H), 1.77 (m, 1H), 1.47-1.59 (m, 4H), 0.96 (t, J=7.4 Hz, 6H).

3-Chloro-4-(2-ethylbutoxy)benzaldehyde (0.05 mmol, 13 mg) and aminoguanidine hydrochloride (0.10 mmol, 10 mg) in EtOH (1 mL) were shaken at 70° C. for 18 hours then concentrated in vacuo. The crude material was dissolved in CH₃CH:H₂O (3:7, 300 μL) and purified by preparative LC/MS. The fractions containing the desired compound were concentrated in vacuo to give the title compound (2052) as a white powder (5 mg, 27%). ¹H NMR (CD₃OD) δ 8.02 (s, 1H), 7.93 (d, J=2.0 Hz, 1H), 7.60 (dd, J=8.6, 2.0 Hz, 1H), 7.11 (d, J=8.6 Hz, 1H), 4.02 (d, J=5.7 Hz, 2H), 1.94 (s, 3H), 1.72 (m, 1H), 1.54 (m, 4H), 0.97 (t, J=7.5 Hz, 6H); HPLC-MS (ammonium acetate) [M+H]⁺=297.1, 299.1.

### Example 52

### 1-(3-Chloro-4-octyloxybenzylideneamino)guanidine Hydrochloride (2053)

1-Iodooctane (1.0 mmol, 240 mg) was used according to GP5 and the crude material was purified using CF5 to give 3-chloro-4-octyloxybenzaidehyde as a white solid (229 mg, 85%). ¹H NMR (CDCl₃) δ 9.84 (s, 1H), 7.90 (d, J=2.0 Hz, 1H), 7.74 (dd, J=8.4, 2.0 Hz, 1H), 7.01 (d, J=8.4 Hz, 1H), 4.11 (t, J=6.5 Hz, 2H), 1.86 (m, 2H), 1.51 (m, 2H), 1.25-1.41 (m, 8H), 0.89 (m, 3H).

3-Chloro-4-octyloxybenzaldehyde (0.85 mmol, 229 mg) and aminoguanidine hydrochloride (0.81 mmol, 89 mg) were used according to GP3 (but without a DCM wash of the precipitate) to give the title compound (2053) as a white powder (196 mg, 63%). ¹H NMR (CD₃OD) δ 8.01 (s, 1H), 7.95 (d, J=2.2 Hz, 1H), 7.62 (dd, J=8.6, 2.2 Hz, 1H), 7.10 (d, J=8.6 Hz, 1H), 4.11 (t, J=6.5 Hz, 2H), 1.84 (m, 2H), 1.53 (m, 2H), 1.26-1.45 (m, 8H), 0.91 (m, 3H); HPLC-MS (ammonium acetate) [M+H]⁺=325.1, 327.1.

### Example 53

### 1-[3-Chloro-4-(2-ethoxy-ethoxy)benzylideneamino])guanidine Acetate (2054)

1-Bromo-2-ethoxyethane (1.0 mmol, 153 mg) was used according to GP5 and the crude material was purified using CF4 to give 3-chloro-4-(2-ethoxy-ethoxy)benzaldehyde as a white solid (28 mg, 12%). ¹H NMR (CDCl₃) δ 9.84 (s, 1H), 7.90 (d, J=2.0 Hz, 1H), 7.74 (dd, J=8.4, 2.0 Hz, 1H), 7.06 (d, J=8.4 Hz, 1H), 4.27 (m, 2H), 3.87 (m, 2H), 3.64 (q, J=7.0 Hz, 2H), 1.24 (t, J=7.0 Hz, 3H).

3-Chloro-4-(2-ethoxy-ethoxy)benzaldehyde (0.12 mmol, 28 mg) and aminoguanidine hydrochloride (0.12 mmol, 12 mg) in EtOH (1 mL) were shaken at 70° C. for 18 hours then concentrated in vacuo. The crude material was dissolved in CH₃CH:H₂O (3:7, 600 μL) and purified by preparative LC/MS. The fractions containing the desired compound were concentrated in vacuo to give the title compound (2054) as a pale yellow oil (22 mg, 52%). ¹H NMR (CD₃OD) δ 8.01 (s, 1H), 7.94 (d, J=2.2 Hz, 1H), 7.61 (dd, J=8.6, 2.2 Hz, 1H), 7.14 (d, J=8.6 Hz, 1H), 4.24 (m, 2H), 3.85 (m, 2H), 3.64 (q, J=7.0 Hz, 2H), 1.22 (t, J=7.0 Hz, 3H); HPLC-MS (ammonium acetate) [M+H]⁺=285.0, 287.0.

### Example 54

### 1-(2-Phenylbenzylideneamino)guanidine Hydrochloride (2055)

Biphenyl-2-carbaldehyde (2.0 mmol, 364 mg) and aminoguanidine hydrochloride (1.9 mmol, 209 mg) were used according to GP6 to give the title compound (2055) as a white powder (440 mg, 80%). ¹H NMR (CD₃OD) δ 8.22 (m, 1H), 8.05 (s, 1H), 7.41-7.54 (m, 5H), 7.30-7.39 (m, 3H); HPLC-MS (ammonium acetate) [M+H]⁺=239.1.

### Example 55

### 1-(3,4-Dichlorophenyl)-1-(propylideneaminoguanidine) Hydrochloride (2056)

1-(3,4-Dichlorophenyl)propan-1-one (2.0 mmol, 406 mg) and aminoguanidine hydrochloride (1.9 mmol, 209 mg) were used according to GP6 to give the title compound (2056) as a white powder (534 mg, 90%). ¹H NMR (CD₃OD) δ 8.13 (d, J=2.2 Hz, 1H), 7.80 (dd, J=8.6, 2.2 Hz, 1H), 7.58 (d, J=8.6 Hz, 1H), 2.82 (q, J=7.7 Hz, 2H), 1.19 (t, J=7.7 Hz, 3H); HPLC-MS (ammonium acetate) [M+H]⁺=259.0, 261.0, 263.0.

### Example 56

### 1-[4-(2-Fluorophenyl)benzylideneamino]guanidine Hydrochloride (2057)

2′-Fluoro-biphenyl-4-carbaldehyde (0.144 mmol, 36 mg) and aminoguanidine hydrochloride (0.13 mmol, 14 mg) in EtOH (2 mL) were heated in a microwave at 120° C. for 10 minutes then cooled to room temperature. Water (20 mL) and NaOH (2M, 5 mL) were added and the product was extracted with EtOAc (2×20 mL). The organic layer was washed with water (10 mL), brine (10 mL), dried over MgSO₄ and filtered. HCl in ether (2 M, 0.5 mL) was added and the solution concentrated. Recrystallisation from MeOH/Et₂O gave the title compound (2057) as a cream powder (12 mg, 25%); ¹H NMR (CD₃OD) δ 8.14 (s, 1H), 7.90 (m, 2H), 7.64 (m, 2H), 7.52 (m, 1H), 7.40 (m, 1H), 7.28 (m, 1H), 7.21 (m, 1H); HPLC-MS (ammonium acetate) [M+H]⁺=257.1.

### Example 57

### 1-[3-(2-Trifluoromethylphenyl)benzylideneamino]guanidine Hydrochloride (2058)

2′-Trifluoromethyl-biphenyl-3-carbaldehyde (0.132 mmol, 33 mg) and aminoguanidine hydrochloride (0.12 mmol, 13 mg) in EtOH (2 mL) were heated in a microwave at 120° C. for 10 minutes then cooled to room temperature. Water (20 mL) and NaOH (2M, 5 mL) were added and the product was extracted with EtOAc (2×20 mL). The organic layer was washed with water (10 mL), brine (10 mL), dried over MgSO₄ and filtered. HCl in ether (2 M, 0.5 mL) was added and the solution concentrated. Recrystallisation from MeOH/Et₂O gave the title compound (2058) as a white powder (9 mg, 19%); ¹H NMR (CD₃OD) δ 8.13 (s, 1H), 7.86 (m, 1H), 7.79 (m, 2H), 7.67 (m, 1H), 7.58 (m, 1H), 7.51 (m, 1H), 7.41 (m, 2H); HPLC-MS (ammonium acetate) [M+H]⁺=307.1.

### Example 58

### 1-(5-Chloro-2,3-dimethoxybenzylideneamino)guanidine Hydrochloride (2059)

5-Chloro-2,3-dimethoxybenzaldehyde (2.0 mmol, 401 mg) and aminoguanidine hydrochloride (1.9 mmol, 210 mg) were used according to GP3 to give the title compound (2059) as a white powder (449 mg, 80%). ¹H NMR (CD₃OD) δ 8.38 (d, J=0.4 Hz, 1H), 7.69 (dd, J=2.5, 0.4 Hz, 1H), 7.11 (d, J=2.5 Hz, 1H), 3.89 (s, 3H), 3.87 (s, 3H); HPLC-MS (ammonium acetate) [M+H]⁺=257.0, 259.0.

### Example 59

### 1-[2-Fluoro-4-(trifluoromethyl)benzylideneamino]guanidine Hydrochloride (2060)

2-Fluoro-4-(trifluoromethyl)benzaldehyde (2.0 mmol, 384 mg) and aminoguanidine hydrochloride (1.9 mmol, 210 mg) were used according to GP3 to give the title compound (2060) as a white powder (478 mg, 88%). ¹H NMR (CD₃OD) δ 8.38 (s, 1H), 8.33 (m, 1H), 7.57 (m, 2H); HPLC-MS (ammonium acetate) [M+H]⁺=249.0.

### Example 60

### 1-[2,4-Bis(trifluoromethyl)benzylideneamino]guanidine Hydrochloride (2061)

2,4-Bis(trifluoromethyl)benzaldehyde (2.0 mmol, 484 mg) and aminoguanidine hydrochloride (1.9 mmol, 210 mg) were used according to GP3 to give the title compound (2061) as a white powder (566 mg, 89%). ¹H NMR (CD₃OD) δ 8.61 (m, 1H), 8.52 (m, 1H), 8.05 (m, 1H), 8.02 (m, 1H); HPLC-MS (ammonium acetate) [M+H]⁺=299.0.

### Example 61

### 1-[2,3-Difluoro-4-(trifluoromethyl)benzylideneamino]guanidine Hydrochloride (2062)

2,3-Difluoro-4-(trifluoromethyl)benzaldehyde (2.0 mmol, 420 mg) and aminoguanidine hydrochloride (1.9 mmol, 210 mg) were used according to GP3 to give the title compound (2062) as a white powder (520 mg, 90%). ¹H NMR (CD₃OD) δ 8.36 (s, 1H), 8.09 (m, 1H), 7.55 (m, 1H); HPLC-MS (ammonium acetate) [M+H]⁺=267.0.

### Example 62

### 1-[3-Fluoro-4-(trifluoromethyl)benzylideneamino]guanidine Hydrochloride (2063)

3-Fluoro-4-(trifluoromethyl)benzaldehyde (2.0 mmol, 384 mg) and aminoguanidine hydrochloride (1.9 mmol, 210 mg) were used according to GP3 to give the title compound (2063) as a white powder (469 mg, 86%). ¹H NMR (CD₃OD) δ 8.15 (s, 1H), 7.92 (m, 1H), 7.71-7.79 (m, 2H); HPLC-MS (ammonium acetate) [M+H]⁺=249.0.

### Example 63

### 1-[3-Nitro-4-(trifluoromethyl)benzylideneamino]guanidine Hydrochloride (2064)

3-Nitro-4-(trifluoromethyl)benzaldehyde (2.0 mmol, 438 mg) and aminoguanidine hydrochloride (1.9 mmol, 210 mg) were used according to GP2 to give the title compound (2064) as a pale yellow powder (493 mg, 83%). ¹H NMR (CD₃OD) δ 8.66 (s, 1H), 8.50 (m, 1H), 8.42 (m, 1H), 8.07 (m, 1H); HPLC-MS (ammonium acetate) [M+H]⁺=276.0.

### Example 64

### 1-[2-Fluoro-3-(trifluoromethyl)benzylideneamino]guanidine Hydrochloride (2065)

2-Fluoro-3-(trifluoromethyl)benzaldehyde (2.0 mmol, 384 mg) and aminoguanidine hydrochloride (1.9 mmol, 210 mg) were used according to GP2 to give the title compound (2065) as a white powder (500 mg, 92%). ¹H NMR (CD₃OD) δ 8.41 (m, 1H), 8.39 (s, 1H), 7.80 (m, 1H), 7.44 (m, 1 H); HPLC-MS (ammonium acetate) [M+H]⁺=249.0.

### Example 65

### 1-[2-Fluoro-5-(trifluoromethyl)benzylideneamino]guanidine Hydrochloride (2066)

2-Fluoro-5-(trifluoromethyl)benzaldehyde (2.0 mmol, 384 mg) and aminoguanidine hydrochloride (1.9 mmol, 210 mg) were used according to GP3 to give the title compound (2066) as a white powder (410 mg, 75%). ¹H NMR (CD₃OD) δ 8.54 (dd, J=6.5, 2.2 Hz, 1H), 8.38 (s, 1H), 7.81 (m, 1H), 7.42 (ap. t, J=9.5 Hz, 1H); HPLC-MS (ammonium acetate) [M+H]⁺=249.0.

### Example 66

### 1-[3-Fluoro-5-(trifluoromethyl)benzylideneamino]guanidine Hydrochloride (2067)

3-Fluoro-5-(trifluoromethyl)benzaldehyde (2.0 mmol, 384 mg) and aminoguanidine hydrochloride (1.9 mmol, 210 mg) were used according to GP3 to give the title compound (2067) as a white powder (458 mg, 84%). ¹H NMR (CD₃OD) δ 8.17 (s, 1H), 7.98 (br. s, 1H), 7.95 (m, 1H), 7.55 (m, 1H); HPLC-MS (ammonium acetate) [M+H]⁺=249.0.

### Example 67

### 1-[4-Fluoro-3-(trifluoromethyl)benzylideneamino]guanidine Hydrochloride (2068)

4-Fluoro-3-(trifluoromethyl)benzaldehyde (2.0 mmol, 384 mg) and aminoguanidine hydrochloride (1.9 mmol, 210 mg) were used according to GP3 to give the title compound (2068) as a white powder (459 mg, 84%). ¹H NMR (CD₃OD) δ 8.21 (dd, J=6.7, 2.2 Hz, 1H), 8.17 (s, 1H), 8.11 (ddd, J=8.6, 4.7, 2.2 Hz, 1H), 7.43 (m, 1H); HPLC-MS (ammonium acetate) [M+H]⁺=249.0.

### Example 68

### 1-[2-Chloro-5-(trifluoromethyl)benzylideneamino]guanidine Hydrochloride (2069)

2-Chloro-5-(trifluoromethyl)benzaldehyde (2.0 mmol, 417 mg) and aminoguanidine hydrochloride (1.9 mmol, 210 mg) were used according to GP3 to give the title compound (2069) as a white powder (486 mg, 85%). ¹H NMR (CD₃OD) δ 8.60 (s, 1H), 8.55 (m, 1H), 7.73 (dd, J=8.6, 2.2 Hz, 1H), 7.69 (m, 1H); HPLC-MS (ammonium acetate) [M+H]⁺=265.0, 267.0.

### Example 69

### 1-[2-Chloro-3-(trifluoromethyl)benzylideneamino]guanidine Hydrochloride (2070)

2-Chloro-3-(trifluoromethyl)benzaldehyde (2.0 mmol, 417 mg) and aminoguanidine hydrochloride (1.9 mmol, 210 mg) were used according to GP3 to give the title compound (2070) as a white powder (518 mg, 90%). ¹H NMR (CD₃OD) δ 8.67 (s, 1H), 8.44 (dd, J=7.9, 1.6 Hz, 1H), 7.88 (dd, J=7.9, 1.0 Hz, 1H), 7.57 (m, 1H); HPLC-MS (ammonium acetate) [M+H]⁺=265.0, 267.0.

### Example 70

### 1-[3-Chloro-2-fluoro-5-(trifluoromethyl)benzylideneamino]guanidine Hydrochloride (2071)

3-Chloro-2-fluoro-5-(trifluoromethyl)benzaldehyde (2.0 mmol, 453 mg) and aminoguanidine hydrochloride (1.9 mmol, 210 mg) were used according to GP3 to give the title compound (2071) as a white powder (527 mg, 86%). ¹H NMR (CD₃OD) δ 8.50 (m, 1H), 8.37 (s, 1H), 7.95 (m, 1H); HPLC-MS (ammonium acetate) [M+H]⁺=283.0, 285.0.

### Example 71

### 1-[(4-Fluoro-1-naphthalen-1-yl)methylideneamino]guanidine Hydrochloride (2072)

4-Fluoro-1-naphthalenecarboxaldehyde (2.0 mmol, 348 mg) and aminoguanidine hydrochloride (1.9 mmol, 210 mg) were used according to GP3 to give the title compound (2072) as a white powder (439 mg, 86%). ¹H NMR (CD₃OD) δ 8.84 (s, 1H), 8.54 (m, 1H), 8.18 (m, 1H), 8.14 (dd, J=8.2, 5.7 Hz, 1H), 7.74 (m, 1H), 7.67 (m, 1H), 7.30 (dd, J=10.2, 8.2 Hz, 1H); HPLC-MS (ammonium acetate) [M+H]⁺=231.0.

### Example 72

### 1-[4-Methoxy-3-(trifluoromethyl)benzylideneamino]guanidine Hydrochloride (2073)

4-Methoxy-3-(trifluoromethyl)benzaldehyde (2.0 mmol, 408 mg) and aminoguanidine hydrochloride (1.9 mmol, 210 mg) were used according to GP3 to give the title compound (2073) as a white powder (313 mg, 55%). ¹H NMR (CD₃OD) δ 8.09 (s, 1H), 8.08 (d, J=2.2 Hz, 1H), 8.00 (dd, J=8.8, 2.2 Hz, 1H), 7.26 (d, J=8.8 Hz, 1H), 3.97 (s, 3H); HPLC-MS (ammonium acetate) [M+H]⁺=261.0.

### Example 73

### 1-[2-Methoxy-5-(trifluoromethyl)benzylideneamino]guanidine Hydrochloride (2074)

2-Methoxy-5-(trifluoromethyl)benzaldehyde (2.0 mmol, 408 mg) and aminoguanidine hydrochloride (1.9 mmol, 210 mg) were shaken at 70° C. for 18 hours then cooled to room temperature. The reaction was concentrated, the crude was dissolved in minimum amount of MeOH, Et₂O was added and the title compound (2074) crystallised out over a couple of days as white crystals (477 mg, 84%). ¹H NMR (CD₃OD) δ 8.50 (s, 1H), 8.37 (d, J=2.4 Hz, 1H), 7.72 (ddd, J=8.8, 2.4, 0.8 Hz, 1H), 7.25 (d, J=8.8 Hz, 1H), 3.98 (s, 3H); HPLC-MS (ammonium acetate) [M+H]⁺=261.0.

### Example 74

### 1-[Naphthalen-2-yl-methylideneamino]guanidine Hydrochloride (2075)

2-Naphthaldehyde (2.0 mmol, 312 mg) and aminoguanidine hydrochloride (1.9 mmol, 210 mg) were used according to GP2 to give the title compound (2075) as a white powder (428 mg, 90%). ¹H NMR (CD₃OD) δ 8.27 (s, 1H), 8.12 (br. s; 1H), 8.08 (dd, J=8.6, 1.8 Hz, 1H), 7.85-7.95 (m, 3H), 7.55 (m, 2H); HPLC-MS (ammonium acetate) [M+H]⁺=213.1.

### Example 75

### 1-[5-Bromo-2-ethoxybenzylideneamino]guanidine Hydrochloride (2076)

5-Bromo-2-ethoxybenzaldehyde (2.0 mmol, 458 mg) and aminoguanidine hydrochloride (1.9 mmol, 210 mg) were used according to GP6 to give the title compound (2076) as a white powder (363 mg, 59%). ¹H NMR (CD₃OD) δ 8.45 (s, 1H), 8.21 (d, J=2.5 Hz, 1H), 7.51 (dd, J=8.8, 2.5 Hz, 1H), 7.00 (d, J=8.8 Hz, 1H), 4.13 (q, J=6.9 Hz, 2H), 1.44 (t, J=6.9 Hz, 3H); HPLC-MS (ammonium acetate) [M+H]⁺=285.0, 287.0.

### Example 76

### 1-[2,4-Dimethylbenzylideneamino]guanidine Hydrochloride (2077)

2,4-Dimethylbenzaldehyde (2.0 mmol, 368 mg) and aminoguanidine hydrochloride (1.9 mmol, 210 mg) were used according to GP3 to give the title compound (2077) as a white powder (342 mg, 79%). ¹H NMR (CD₃OD) δ 8.40 (s, 1H), 7.85 (d, J=8.4 Hz, 1H), 7.07 (m, 2H), 2.44 (s, 3H), 2.33 (s, 3H); HPLC-MS (ammonium acetate) [M+H]⁺=191.1.

### Example 77

### 1-[4-Chloro-3-nitrobenzylideneamino]guanidine Hydrochloride (2078)

4-Chloro-3-nitrobenzaldehyde (2.0 mmol, 371 mg) and aminoguanidine hydrochloride (1.9 mmol, 210 mg) were used according to GP2 to give the title compound (2078) as a pale yellow powder (487 mg, 92%). ¹H NMR (CD₃OD) δ 8.44 (d, J=2.0 Hz, 1H), 8.16 (s, 1H), 8.02 (dd, J=8.4, 2.0 Hz, 1H), 7.73 (d, J=8.4 Hz, 1H); HPLC-MS (ammonium acetate) [M+H]⁺=242.0, 244.0.

### Example 78

### 1-(4-Benzyloxy-2-hydroxybenzylideneamino)guanidine Hydrochloride (3001)

4-Benzyloxy-2-hydroxybenzaldehyde (2.0 mmol, 456 mg) was used according to GP7 to give the title compound (3001) as a white powder (358 mg, 64%). ¹H NMR (CD₃OD) δ 8.39 (s, 1H), 7.72 (d, J=8.8 Hz, 1H), 7.48-7.34 (m, 5H), 6.63 (dd, J=8.8, 2.5 Hz, 1H), 6.57 (d, J=2.4 Hz, 1H), 5.14 (s, 2H); HPLC-MS (ammonium bicarbonate) [M+H]⁺=285.2.

### Example 79

### 1-[(1H-Indol-5-yl)methylideneamino]guanidine Hydrochloride (3002)

Indole-5-carboxaldehyde (2.0 mmol, 290 mg) was used according to GP8 to give the title compound (3002) as a red powder (266 mg, 65%). ¹H NMR (CD₃OD) δ 8.23 (s, 1H), 7.95 (d, J=1.4 Hz, 1H), 7.73 (dd, J=8.6, 1.5 Hz, 1H), 7.49 (d, J=8.6 Hz, 1H), 7.34 (d, J=3.1 Hz, 2H), 6.57 (dd, J=3.1, 0.8 Hz, 1H); HPLC-MS (ammonium bicarbonate) [M+H]⁺=202.2.

### Example 80

### 1-(4-Butoxybenzylideneamino)guanidine Hydrochloride (3003)

4-Butoxybenzaldehyde (2.0 mmol, 356 mg) was used according to GP7 to give the title compound (3003) as a white powder (355 mg, 76%). ¹H NMR (CD₃OD) δ 8.14 (s, 1H), 7.78 (m, 2H), 7.01 (m, 2H),-4.04 (t, J=6.4 Hz, 2H), 1.80 (m, 2H), 1.55 (m, 2H), 1.03 (t, J=7.2 Hz, 3H); HPLC-MS (ammonium bicarbonate) [M+H]⁺=235.2.

### Example 81

### 1-(4-Cyanobenzylideneamino)guanidine Hydrochloride (3004)

4-Cyanobenzaldehyde (2.0 mmol, 262 mg) was used according to GP8 to give the title compound (3004) as a white powder (343 mg, 92%). ¹H NMR (CD₃OD) δ 8.25 (s, 1H), 8.05 (m, 2H), 7.86 (m, 2H); HPLC-MS (ammonium bicarbonate) [M+H]⁺=188.1.

### Example 82

### 1-(2,5-Dimethoxybenzylideneamino)guanidine Hydrochloride (3005)

2,5-Dimethoxybenzaldehyde (2.0 mmol, 332 mg) was used according to GP7 to give the title compound (3005) as a yellow powder (355 mg, 69%). ¹H NMR (CD₃OD) δ 8.53 (s, 1H), 7.64 (dd, J=2.3, 0.6 Hz, 1H), 7.06 (m, 2H), 3.90 (s, 3H), 3.87 (s, 3H); HPLC-MS (ammonium bicarbonate) [M+H]⁺=223.2.

### Example 83

### 1-(2-Benzyloxy-3-methoxybenzylideneamino)guanidine Hydrochloride (3006)

2-Benzyloxy-3-methoxybenzaldehyde (2.0 mmol, 484 mg) was used according to GP7 to give the title compound (3006) as a white powder (460 mg, 69%). ¹H NMR (CD₃OD) δ 8.35 (s, 1H), 7.63 (dd, J=6.8, 2.3 Hz, 1H), 7.47-7.35 (m, 5H), 7.19 (m, 2H), 5.13 (s, 2H), 3.98 (s, 3H); HPLC-MS (ammonium bicarbonate) [M+H]⁺=299.3.

### Example 84

### 1-[1-(2-Methoxy-naphthalen-1-yl)methylideneamino]guanidine Hydrochloride (3007)

2-Methoxy-1-naphthaldehyde (2.0 mmol, 372 mg) was used according to GP7 to give the title compound (3007) as a pale green powder (275 mg, 49%). ¹H NMR (CD₃OD) δ 8.94 (d, J=8.6 Hz, 1H), 8.88 (s, 1H), 7.99 (d, J=8.9 Hz, 1H), 7.86 (d, J=7.9 Hz, 1H), 7.62 (m, 1H), 7.45 (m, 2H), 4.03 (s, 3H); HPLC-MS (ammonium bicarbonate) [M+H]⁺=243.2.

### Example 85

### 1-(4-Hydroxy-3-methoxy-5-nitrobenzylideneamino)guanidine Hydrochloride (3008)

4-Hydroxy-3-methoxy-5-nitrobenzaldehyde (2.0 mmol, 394 mg) was used according to GP7 to give the title compound (3008) as a yellow powder (509 mg, 88%). ¹H NMR (DMSO) δ 7.69 (s, 1H), 7.43 (s, 1H), 7.36 (s, 1H), 3.50 (s, 3H), 2.97 (m, 3H, NH); HPLC-MS (ammonium bicarbonate) [M+H]⁺=254.2.

### Example 86

### 1-(3,4-Dihydroxybenzylideneamino)guanidine Hydrochloride (3009)

3,4-Dihydroxybenzaldehyde (2.0 mmol, 276 mg) was used according to GP8 to give the title compound (3009) as a pale yellow powder (375 mg, 81%). ¹H NMR (CD₃OD) δ 7.99 (s, 1H), 7.30 (d, J=1.9 Hz, 1H), 7.12 (dd, J=8.2, 1.9, 1H), 6.85 (d, J=8.2, 1H); HPLC-MS (ammonium bicarbonate) [M+H]⁺=195.1.

### Example 87

### 1-(3-Bromobenzylideneamino)guanidine Hydrochloride (3010)

3-Bromobenzaldehyde (2.0 mmol, 370 mg) was used according to GP8 to give the title compound (3010) as a white powder (363 mg, 66%). ¹H NMR (CD₃OD) δ 8.17 (s, 1H), 8.12 (ap. t, J=1.6 Hz, 1H), 7.78 (ap. dt, J=7.8, 1.2 Hz, 1H), 7.64 (ddd, J=8.0, 2.0, 1.0 Hz, 1H), 7.41 (ap. t, J=8.1 Hz, 1H); HPLC-MS (ammonium bicarbonate) [M+H]⁺=241.1, 243.1.

### Example 88

### 1-(3,5-Dibromobenzylideneamino)guanidine Hydrochloride (3011)

3,5-Dibromobenzaldehyde (2.0 mmol, 527 mg) was used according to GP7 to give the title compound (3011) as a white powder (488 mg, 68%). ¹H NMR (CD₃OD) δ 8.11 (s, 1H), 8.08 (d, J=1.7 Hz, 2H), 7.86 (ap. t, J=1.7 Hz, 1H); HPLC-MS (ammonium bicarbonate) [M+H]⁺=271.2, 273.2.

### Example 89

### 1-[1-(3,4-Dichlorophenyl)ethylideneamino]guanidine Hydrochloride (3012)

3,4-Dichloroacetophenone (2.0 mmol, 378 mg) was used according to GP2 to give the title compound (3012) as a white powder (368 mg, 66%). ¹H NMR (CD₃OD) δ 8.16 (ap. t, J=1.5 Hz, 1H), 7.85 (dt, J=8.6, 2.1 Hz, 1H), 7.61 (dd, J=8.6, 1.5 Hz, 1H), 2.42 (s, 3H); HPLC-MS (ammonium bicarbonate) [M+H]⁺=245.1, 247.1, 249.1.

### Example 90

### 1-(4-n-Hexyloxybenzylideneamino)guanidine Hydrochloride (3013)

4-n-hexyloxybenzaldehyde (2.0 mmol, 412 mg) was used according to GP7 to give the title compound (3013) as a white powder (386 mg, 65%). ¹H NMR (CD₃OD) δ 8.12 (s, 1H), 7.78 (dd, J=6.9, 1.9 Hz, 2H), 7.02 (dd, J=6.8, 1.9 Hz, 2H), 4.08 (t, J=6.4 Hz, 2H), 1.84 (m, 2H), 1.54 (m, 2H), 1.43 (m, 4H), 0.99 (t, J=7.2 Hz, 3H); HPLC-MS (ammonium bicarbonate) [M+H]⁺=263.3.

### Example 91

### 1-(3,4-Dibenzyloxybenzylideneamino)guanidine Hydrochloride (3014)

3,4-Dibenzyloxybenzaldehyde (2.0 mmol, 636 mg) was used according to GP7 to give the title compound (3014) as a white powder (583 mg, 72%). ¹H NMR (CD₃OD) δ 8.05 (s, 1H), 7.67 (d, J=1.9 Hz, 1H), 7.52-7.45 (m, 4H), 7.41-7.31 (m, 6H), 7.27 (dd, J=8.2, 1.9 Hz, 1H), 7.09 (d, J=8.4 Hz, 1H), 5.20 (s, 2H), 5.19 (s, 2H); HPLC-MS (ammonium bicarbonate) [M+H]⁺=375.3.

### Example 92

### 1-[(6-Bromobenzo[1,3]dioxol-5-yl)methylideneamino]guanidine Hydrochloride (3015)

6-Bromopiperonal (2.0 mmol, 458 mg) was used according to GP8 to give the title compound (3015) as a white powder (539 mg, 84%). ¹H NMR (CD₃OD) δ 8.52 (s, 1H), 7.76 (s, 1H), 7.18 (s, 1H), 6.13 (s, 2H); HPLC-MS (ammonium bicarbonate) [M+H]⁺=285.2, 287.2.

### Example 93

### 1-[1-(4-Bromophenyl)ethylideneamino]guanidine Hydrochloride (3016)

4-Bromoacetophenone (2.0 mmol, 398 mg) was used according to GP3 to give the title compound (3016) as a white powder (455 mg, 79%). ¹H NMR (CD₃OD) δ 7.88 (m, 2H), 7.63 (m, 2H), 2.42 (s, 3H); HPLC-MS (ammonium bicarbonate) [M+H]⁺=255.1, 257.1.

### Example 94

### 1-[1-(3-Methylphenyl)ethylideneamino]guanidine Hydrochloride (3017)

3-Methylacetophenone (2.0 mmol, 268 mg) was used according to GP2 to give the title compound (3017) as a white powder (316 mg, 70%). ¹H NMR (CD₃OD) δ 7.78 (br. s, 1H), 7.72 (m, 1H), 7.36 (ap. t, J=7.6 Hz, 1H), 7.31 (m, 1H), 2.45 (s, 3H), 2.42 (s, 3H); HPLC-MS (ammonium bicarbonate) [M+H]⁺=191.2.

### Example 95

### 1-(3-Methylbenzylideneamino)guanidine Hydrochloride (3018)

3-Methylbenzaldehyde (2.0 mmol, 240 mg) was used according to GP7 to give the title compound (3018) as a pale yellow powder (259 mg, 62%). ¹H NMR (CD₃OD) δ 8.16 (s, 1H), 7.70 (br. s, 1H), 7.63 (d, J=7.6 Hz, 1H), 7.37 (ap. t, J=7.5 Hz, 1H), 7.32 (m, 1H), 2.43 (s, 3H); HPLC-MS (ammonium bicarbonate) [M+H]⁺=177.2.

### Example 96

### 1-(3,4-Dimethylbenzylideneamino)guanidine Hydrochloride (3019)

3,4-Dimethylbenzaldehyde (2.0 mmol, 268 mg) was used according to GP7 to give the title compound (3019) as a white powder (355 mg, 78%). ¹H NMR (CD₃OD) δ 8.12 (s, 1H), 7.65 (br. s, 1H), 7.55 (dd, J=7.8, 1.8 Hz, 1H), 7.25 (d, J=7.6 Hz, 1H), 2.37 (s, 3H), 2.36 (s, 3H); HPLC-MS (ammonium bicarbonate) [M+H]⁺=191.2.

### Example 97

### 1-[1-(4-Ethylphenyl)ethylideneamino]guanidine Hydrochloride (3020)

4-Ethylacetophenone (2.0 mmol, 296 mg) was used according to GP2 to give the title compound (3020) as a white powder (209 mg, 44%). ¹H NMR (CD₃OD) δ 7.86 (m, 2H), 7.32 (m, 2H), 2.74 (q, J=7.6 Hz, 2H), 2.41 (s, 3H), 1.30 (t, J=7.6 Hz, 3H); HPLC-MS (ammonium bicarbonate) [M+H]⁺=205.3.

### Example 98

### 1-[1-(3,4-Dimethylphenyl)ethylideneamino]guanidine Hydrochloride (3021)

3,4-Dimethylacetophenone (2.0 mmol, 296 mg) was used according to GP2 to give the title compound (3021) as a white powder (415 mg, 87%). ¹H NMR (CD₃OD) δ 7.74 (br. s, 1H), 7.64 (m, 1H), 7.23 (d, J=8.0, 1H), 2.39 (s, 3H), 2.37 (s, 3H), 2.35 (s, 3H); HPLC-MS (ammonium bicarbonate) [M+H]⁺=205.3.

### Example 99

### 1-(4-n-pentylbenzylideneamino)guanidine Hydrochloride (3022)

4-n-pentylbenzaldehyde (2.0 mmol, 362 mg) was used according to GP8 to give the title compound (3022) as a white powder (247 mg, 47%). ¹H NMR (CD₃OD) δ 8.17 (s, 1H), 7.76 (m, 2H), 7.32 (d, J=8.2 Hz, 2H), 2.70 (t, J=7.5 Hz, 2H), 1.69 (m, 2H), 1.40 (m, 4H), 0.96 (t, J=7.0 Hz, 3H); HPLC-MS (ammonium bicarbonate) [M+H]⁺=233.3.

### Example 100

### 1-[1-(4-n-Heptylphenyl)ethylideneamino]guanidine Hydrochloride (3023)

4-n-Hexylacetophenone (2.0 mmol, 408 mg) was used according to GP3 to give the title compound (3023) as a white powder (162 mg, 29%). ¹H NMR (CD₃OD) δ 7.86 (m, 2H), 7.30 (d, J=8.6 Hz, 2H), 2.71 (t, J=7.4 Hz, 2H), 2.42 (s, 3H), 1.69 (m, 2H), 1.44-1.35 (m, 6H), 0.96 (t, J=7.0 Hz, 3H); HPLC-MS (ammonium bicarbonate) [M+H]⁺=261.3.

### Example 101

### 1-[1-(5,6,7,8-Tetrahydronaphthalen-2-yl)ethylideneamino]guanidine Hydrochloride (3024)

6-Acetyl-1,2,3,4-tetrahydronaphthalene (2.0 mmol, 348 mg) was used according to GP2 to give the title compound (3024) as a white powder (374 mg, 70%). ¹H NMR (CD₃OD) δ 7.64 (m, 1H), 7.62 (br. s, 1H), 7.14 (d, J=8.0 Hz, 1H), 2.89-2.82 (m, 4H), 2.39 (s, 3H), 1.89-1.82 (m, 4H); HPLC-MS (ammonium bicarbonate) [M+H]⁺=231.3.

### Example 102

### 1-(4-Ethylbenzylideneamino)guanidine Hydrochloride (3025)

4-Ethylbenzaldehyde (2.0 mmol, 268 mg) was used according to GP7 to give the title compound (3025) as a pale yellow oil (272 mg, 60%). ¹H NMR (CD₃OD) δ 8.13 (s, 1H), 7.74 (d, J=8.2 Hz, 2H), 7.31 (d, J=8.3 Hz, 2H), 2.71 (q, J=7.6 Hz, 2H), 1.27 (t, J=7.6 Hz, 3H); HPLC-MS (ammonium bicarbonate) [M+H]⁺=191.2.

### Example 103

### 1-[1-(2-Bromophenyl)ethylideneamino]guanidine Hydrochloride (3026)

2-Bromoacetophenone (2.0 mmol, 398 mg) was used according to GP2 to give the title compound (3026) as a pale pink powder (355 mg, 61%) in a 7:3 mixture of two isomers. Major isomer: ¹H NMR (CD₃OD) δ 7.71 (dd, J=8.0, 0.8 Hz, 1H), 7.49 (m, 2H), 7.39 (m, 1H), 2.43 (s, 3H); Minor isomer: ¹H NMR (CD₃OD) δ 7.84 (dd, J=8.0, 0.9 Hz, 1H), 7.62 (ap. dt, J=7.4, 0.8 Hz, 1H), 7.51 (m, 1H), 7.37 (m, 1H), 2.39 (s, 3H); HPLC-MS (ammonium bicarbonate) [M+H]⁺=255.2, 257.2 (both isomers co-eluted).

### Example 104

### 1-{1-[3-(Trifluoromethyl)phenyl]ethylideneamino}guanidine Hydrochloride (3027)

3-(Trifluoromethyl)acetophenone (2.0 mmol, 376 mg) was used according to GP3 to give the title compound (3027) as a white powder (356 mg, 64%). ¹H NMR (CD₃OD) δ 8.24 (br. s, 1H), 8.22 (d, J=8.0 Hz, 1H), 7.79 (dd, J=7.6, 0.7 Hz, 1H), 7.69 (dt, J=7.8, 0.6 Hz, 1H), 2.48 (s, 3H); HPLC-MS (ammonium bicarbonate) [M+H]⁺=245.2.

### Example 105

### 1-{1-[3,5-Bis-(trifluoromethyl)phenyl]ethylideneamino}guanidine Hydrochloride (3028)

3,5-Bis-(trifluoromethyl)acetophenone (2.0 mmol, 512 mg) was used according to GP3 to give the title compound (3028) as a white powder (606 mg, 87%). ¹H NMR (CD₃OD) δ 8.54 (s, 2H), 8.08 (s, 1H), 2.53 (s, 3H); HPLC-MS (ammonium bicarbonate) [M+H]⁺=313.2.

### Example 106

### 1-[1-(2,5-Dimethoxyphenyl)ethylideneamino]guanidine Hydrochloride (3029)

2′,5′-Dimethoxyacetophenone (2.0 mmol, 360 mg) was used according to GP2 to give the title compound (3029) as a pale yellow powder (402 mg, 75%) in a ca. 4:1 mixture of two isomers. Major isomer: ¹H NMR (CD₃OD) δ 7.07-7.03 (m, 3H), 3.88 (s, 3H), 3.84 (s, 3H), 2.37 (s, 3H); Minor isomer: ¹H NMR (CD₃OD) δ 7.16 (br. s, 1H), 7.13 (d, J=3.1 Hz, 1H), 6.81 (d, J=2.9 Hz, 1H), 3.87 (s, 3H), 3.85 (s, 3H), 2.33 (s, 3H); HPLC-MS (ammonium bicarbonate) [M+H]⁺=237.3 (both isomers co-eluted).

### Example 107

### 1-[1-(2-Hydroxy-4-methoxyphenyl)ethylideneamino]guanidine Hydrochloride (3030)

2′-Hydroxy-4′-methoxyacetophenone (2.0 mmol, 332 mg) was used according to GP3 to give the title compound (3030) as a white powder (473 mg, 92%) in a 9:1 mixture of two isomers. Major isomer: ¹H NMR (CD₃OD) δ 7.49 (d, J=8.8 Hz, 1H), 6.49 (dd, J=8.8, 2.5 Hz, 1H), 6.45 (d, J=2.5 Hz, 1H), 3.79 (s, 3H), 2.38 (s, 3H); Minor isomer: ¹H NMR (CD₃OD) δ 7.78 (d, J=8.9 Hz, 1H), 6.49 (dd, J=8.8, 2.5 Hz, 1H), 6.41 (d, J=2.5 Hz, 1H), 3.83 (s, 3H), 2.54 (s, 3H); HPLC-MS (ammonium bicarbonate) [M+H]⁺=223.3 (both isomers co-eluted).

### Example 108

### 1-[1-(4-Benzyloxy-2-hydroxy-3-methylphenyl)ethylideneamino]guanidine Hydrochloride (3031)

4′-Benzyloxy-2′-hydroxy-3′-methylacetophenone (2.0 mmol, 512 mg) was used according to GP3 to give the title compound (3031) as a white powder (586 mg, 84%). ¹H NMR (CD₃OD) δ 7.50 (m, 3H), 7.45 (m, 2H), 7.38 (m, 1H), 6.71 (d, J=9.0 Hz, 1H), 5.21 (s, 2H), 2.49 (s, 3H), 2.22 (s, 3H); HPLC-MS (ammonium bicarbonate) [M+H]⁺=313.3.

### Example 109

### 1-[1-(Benzo[1,3]dioxol-5-yl)ethylideneamino]guanidine Hydrochloride (3032)

3′,4′-(Methylenedioxy)acetophenone (2.0 mmol, 328 mg) was used according to GP3 to give the title compound (3032) as a pale yellow powder (478 mg, 93%). ¹H NMR (CD₃OD) δ 7.60 (d, J=1.7 Hz, 1H), 7.42 (dd, J=8.2, 1.7 Hz, 1H), 6.91 (d, J=8.2 Hz, 1H), 6.06 (s, 2H), 2.38 (s, 3H); HPLC-MS (ammonium bicarbonate) [M+H]⁺=221.2.

### Example 110

### 1-(3,4-Dichlorobenzylideneamino)guanidine Hydrochloride (1045)

3,4-Dichlorobenzaldehyde (4.0 mmol, 700 mg) was used according to GP7 to give the title compound (1045) as a white powder (695 mg, 65%). ¹H NMR (CD₃OD) δ 8.09 (s, 1H), 8.05 (d, J=1.9 Hz, 1H), 7.69 (dd, J=8.4, 1.9 Hz, 1H), 7.58 (d, J=8.4 Hz, 1H); HPLC-MS (ammonium acetate) [M+H]⁺=231.1, 233.1, 235.1.

### Example 111

### 1-[1-(4-Dimethylaminophenyl)pentylideneamino]guanidine (3035)

1-(4-Dimethylaminophenyl)-pentan-1-one (0.5 mmol, 102 mg) was used according to GP8 to give a crude mixture which was purified by prep. HPLC. The desired combined fractions were concentrated, diluted with 20% Na₂CO₃ solution and extracted with EtOAc. The organic phase was dried over Na₂SO₄, filtered and concentrated in vacuo to afford the title compound (3035) as a white powder (32 mg, 25%) in a ca. 3:1 mixture of two isomers. Major isomer: ¹H NMR (CD₃OD) δ 7.31 (m, 2H), 6.84 (m, 2H), 3.02 (s, 6H), 2.63 (t, J=7.2 Hz, 2H), 1.52-1.34 (m, 4H), 0.94 (t, J=7.2 Hz, 3H); Minor isomer: ¹H NMR (CD₃OD) δ 7.69 (m, 2H), 6.79 (m, 2H), 3.30 (m, 2H), 3.01 (s, 6H), 1.50-1.35 (m, 4H), 0.97 (t, J=7.2 Hz, 3H); HPLC-MS (ammonium acetate) [M+H]⁺=262.3 (both isomers co-eluted).

### Example 112

### 1-{4-[Ethyl-(2-hydroxyethyl)amino]-2-methylbenzylideneamino}guanidine Hydrochloride (3036)

4-[Ethyl-(2-hydroxyethyl)amino]-2-methylbenzaldehyde (2.0 mmol, 415 mg) and aminoguanidine Hydrochloride (2.0 mmol, 220 mg) were used according to GP7. The crude material was purified on the CombiFlash using method CF6 to give the title compound (30361) as a yellow powder (256 mg, 44%). ¹H NMR (CD₃OD) δ 8.32 (s, 1H), 7.83 (d, J=8.8 Hz, 1H), 6.68 (dd, J=8.8, 2.7 Hz, 1H), 6.60 (d, J=2.7 Hz, 1H), 3.77 (t, J=6.5 Hz, 2H), 3.55 (t, J=6.2 Hz, 2H), 3.54 (q, J=7.0 Hz, 2H), 2.48 (s, 3H), 1.23 (t, J=7.0 Hz, 3H); HPLC-MS (ammonium bicarbonate) [M+H]⁺=264.3.

### Example 113

### 1-(4-Diethylamino-2-hydroxybenzylideneamino)guanidine Hydrochloride (3037)

4-Diethylamino-2-hydroxybenzaldehyde (2.0 mmol, 386 mg) was used according to GP7 to give the title compound (3037) as a pink powder (538 mg, 94%). ¹H NMR (CD₃OD) δ 8.25 (s, 1H), 7.46 (d, J=8.9 Hz, 1H), 6.37 (dd, J=8.9, 2.5 Hz, 1H), 6.21 (d, J=2.5 Hz, 1H), 3.47 (q, J=7.0 Hz, 4H), 1.24 (t, J=7.0 Hz, 6H); HPLC-MS (ammonium acetate) [M+H]⁺=250.2.

### Example 114

### 1-(4-Diethylaminobenzylideneamino)guanidine Hydrochloride (3038)

4-Diethylaminobenzaldehyde (2.0 mmol, 354 mg) was used according to GP8 to give the title compound (3038) as a pale yellow powder (285 mg, 53%). ¹H NMR (CD₃OD) δ 8.01 (s, 1H), 7.65 (d, J=8.4 Hz, 2H), 6.78 (d, J=8.1 Hz, 2H), 3.50 (q, J=7.0 Hz, 4H), 1.24 (t, J=7.0 Hz, 6H); HPLC-MS (ammonium acetate) [M+H]⁺=234.2.

### Example 115

### 1-[1-(4-Piperidin-1-yl-phenyl)ethylideneamino]guanidine Hydrochloride (3039)

4′-Piperidinoacetophenone (2.0 mmol, 406 mg) was used according to GP3 to give the title compound (3039) as a pale yellow powder (493 mg, 84%). ¹H NMR (CD₃OD) δ 7.83 (d, J=8.8 Hz, 2H), 7.01 (d, J=8.9 Hz, 2H), 3.35 (m, 4H), 2.37 (s, 3H), 1.76-1.70 (m, 6H); HPLC-MS (ammonium acetate) [M+H]⁺=260.2.

### Example 116

### 1-{4-[Methyl-(2-cyanoethyl)amino]benzylideneamino}guanidine Hydrochloride (3040)

3-[(4-Formylphenyl)-methylamino]propionitrile (2.0 mmol, 376 mg) and aminoguanidine hydrochloride (2.0 mmol, 220 mg) were used according to GP7 to give the title compound (3040) as a pale yellow powder (509 mg, 91%). ¹H NMR (CD₃OD) δ 8.07 (s, 1H), 7.72 (d, J=8.4 Hz, 2H), 6.88 (d, J=8.4 Hz, 2H), 3.84 (t, J=6.4 Hz, 2H), 3.15 (s, 3H), 2.79 (t, J=6.5 Hz, 2H); HPLC-MS (ammoniumn acetate) [M+H]⁺=245.2.

### Example 117

### 1-{4-[Methyl-(2-hydroxyethyl)amino]benzylideneamino}guanidine Hydrochloride (3041)

4-[Methyl-(2-hydroxyethyl)amino]benzaldehyde (2.0 mmol, 358 mg) and aminoguanidine hydrochloride (2.0 mmol, 220 mg) were used according to GP7 to give the title compound (3041) as a pale yellow powder (320 mg, 59%). ¹H NMR (CD₃OD) δ 8.00 (s, 1H), 7.65 (d, J=8.0 Hz, 2H), 6.82 (d, J=8.0 Hz, 2H), 3.78 (t, J=6.4 Hz, 2H), 3.59 (t, J=6.4 Hz, 2H), 3.11 (s, 3H); HPLC-MS (ammonium acetate) [M+H]⁺=236.2

### Example 118

### 1-(4-Di-n-butylaminobenzylideneamino)guanidine Hydrochloride (3042)

4-Di-n-butylaminobenzaldehyde (2.0 mmol, 466 mg) was used according to GP7 to give the title compound (3042) as a yellow powder (458 mg, 70%). ¹H NMR (CD₃OD) δ 7.99 (s, 1H), 7.62 (d, J=8.8 Hz, 2H), 6.72 (d, J=8.8 Hz, 2H), 3.40 (t, J=7.6 Hz, 4H), 1.65-1.59 (m, 4H), 1.46-1.39 (m, 4H), 1.02 (t, J=7.2 Hz, 6H); HPLC-MS (ammonium acetate) [M+H]⁺=290.2.

### Example 119

### 1-(2-Methoxy-4-N,N-diethylaminobenzylideneamino)guanidine Hydrochloride (3043)

2-Methoxy-4-N,N-diethylaminobenzaldehyde (1.0 mmol, 207 mg) was used according to GP8 to give the title compound (3043) as a yellow powder (152 mg, 57%) in a ca. 9:1 mixture of two isomers. Major isomer: ¹H NMR (CD₃OD) δ 8.38 (s, 1H), 7.86 (d, J=9.0 Hz, 1H), 6.42 (m, 1H), 6.26 (s, 1H), 3.92 (s, 3H), 3.51 (q, J=6.8 Hz, 4H), 1.19 (t, J=7.0 Hz, 6H); Minor isomer: ¹H NMR (CD₃OD) δ 7.63 (s, 1H), 7.34 (d, J=8.6 Hz, 1H), 6.44 (m, 1H), 6.28 (s, 1H), 3.96 (s, 3H), 3.51 (q, J=7.2 Hz, 4H), 0.92 (t, J=7.3 Hz, 6H); HPLC-MS (ammonium acetate) [M+H]⁺=264.2 (both isomers co-eluted).

### Example 120

### 1-(3-Cyanobenzylideneamino)guanidine Hydrochloride (4001)

3-Cyanobenzaldehyde (2.0 mmol, 260 mg) and aminoguanidine hydrochloride (2.0 mmol, 220 mg) were used according to GP3 to give the title compound (4001) as a powder (250 mg, 56%). ¹H NMR (CD₃OD) δ 8.26 (t, J=1.5 Hz, 1H), 8.19 (s, 1H), 8.07 (m, 1H), 7.77 (m, 1H), 7.62 (t, J=7.8 Hz, 1H); HPLC-MS (ammonium acetate) [M+H]⁺=188.1.

### Example 121

### 1-[(4-Trifluoromethyl)benzylideneamino]guanidine Hydrochloride (4002)

4-(Trifluoromethyl)benzaldehyde (2.0 mmol, 250 mg) and aminoguanidine hydrochloride (2.0 mmol, 220 mg) were used according to GP3 to give the title compound (4002) as a powder (400 mg, 75%). ¹H NMR (CD₃OD) δ 8.23 (s, 1H), 8.01 (br. d, J=8.2 Hz, 2H), 7.72 (br. d, J=8.3 Hz, 2H); HPLC-MS (ammonium acetate) [M+H]⁺=231.1.

### Example 122

### 1-(2,4-Dimethoxybenzylideneamino)guanidine Hydrochloride (4003)

2,4-Dimethoxybenzaldehyde (2.0 mmol, 332 mg) and aminoguanidine hydrochloride (2.0 mmol, 220 mg) were used according to GP3 to give the title compound (4003) as a powder (300 mg, 58%). ¹H NMR (CD₃OD) δ 8.39 (s, 1H), 7.95 (m, 1H), 6.59 (m, 2H), 3.87 (s, 3H), 3.85 (s, 3H); HPLC-MS (ammonium acetate) [M+H]⁺=223.1.

### Example 123

### 1-(2,3-Dimethoxybenzylideneamino)guanidine Hydrochloride (4004)

2,3-Dimethoxybenzaldehyde (2.0 mmol, 332 mg) and aminoguanidine hydrochloride (2.0 mmol, 220 mg) were used according to GP3 to give the title compound (4004) as a powder (370 mg, 72%). ¹H NMR (CD₃OD) δ 8.45 (s, 1H), 7.62 (m, 1H), 7.11 (m, 2H), 3.88 (s, 3H), 3.87 (s, 3H); HPLC-MS (ammonium acetate) [M+H]⁺=223.1.

### Example 124

### 1-(4-Ethoxybenzylideneamino)guanidine Hydrochloride (4005)

4-Ethoxybenzaldehyde (2.0 mmol, 300 mg) and aminoguanidine hydrochloride (2.0 mmol, 220 mg) were used according to GP3 to give the title compound (4005) as a powder (290 mg, 60%). ¹H NMR (CD₃OD) δ 8.05 (s, 1H), 7.71 (m, 2H), 6.96 (m, 2H), 4.08 (q, J=7.0 Hz, 2H), 1.40 (t, J=7.0 Hz, 3H); HPLC-MS (ammonium acetate) [M+H]⁺=207.2.

### Example 125

### 1-(4-n-Propoxybenzylideneamino)guanidine Hydrochloride (4006)

4-n-Propoxybenzaldehyde (2.0 mmol, 328 mg) and aminoguanidine hydrochloride (2.0 mmol, 220 mg) were used according to GP3 to give the title compound (4006) as a powder (250 mg, 49%). ¹H NMR (CD₃OD) δ 8.05 (s, 1H), 7.72 (m, 2H), 6.96 (m, 2H), 3.99 (t, J=6.5 Hz, 2H), 1.80 (dt, J=7.4, 6.7 Hz, 2H), 1.05 (t, J=7.4 Hz, 3H); HPLC-MS (ammonium acetate) [M+H]⁺=221.1.

### Example 126

### 1-(2,3,6-Trichlorobenzylideneamino)guanidine Hydrochloride (4007)

2,3,6-Trichlorobenzaldehyde (2.0 mmol, 209 mg) and aminoguanidine hydrochloride (2.0 mmol, 220 mg) were used according to GP3 to give the title compound (4007) as a powder (470 mg, 78%). ¹H NMR (CD₃OD) δ 8.20 (s, 1H), 7.81 (d, J=7.8 Hz, 1H), 7.68 (d, J=7.8 Hz, 1H); HPLC-MS (ammonium acetate) [M+H]⁺=265.0.

### Example 127

### 1-(4-Chlorobenzylideneamino)guanidine Hydrochloride (4008)

4-Chlorobenzaldehyde (2.0 mmol, 281 mg) and aminoguanidine hydrochloride (2.0 mmol, 220 mg) were used according to GP3 to give the title compound (4008) as a powder (380 mg, 82%). ¹H NMR (CD₃OD) δ 8.12 (s, 1H), 7.79 (m, 2H), 7.44 (m, 2H); HPLC-MS (ammonium acetate) [M+H]⁺=197.1.

### Example 128

### 1-(5-Bromo-2-fluorobenzylideneamino)guanidine Hydrochloride (4009)

5-Bromo-2-fluorobenzaldehyde (2.0 mmol, 406 mg) and aminoguanidine hydrochloride (2.0 mmol, 220 mg) were used according to GP3 to give the title compound (4009) as a powder (410 mg, 69%). ¹H NMR (CD₃OD) δ 8.34 (dd, J=6.5, 2.6 Hz, 1H), 8.30 (s, 1H), 7.60 (ddd, J=8.8, 4.7, 2.6, 1H), 7.15 (dd, J=10.2 Hz, J=8.8 Hz, 1H); HPLC-MS (ammonium acetate) [M+H]⁺=259.0.

### Example 129

### 1-(2-Bromo-5-fluorobenzylideneamino)guanidine Hydrochloride (4010)

2-Bromo-5-fluorobenzaldehyde (2.0 mmol, 406 mg) and aminoguanidine hydrochloride (2.0 mmol, 220 mg) were used according to GP3 to give the title compound (4010) as a powder (450 mg, 76%). ¹H NMR (CD₃OD) δ 8.50 (d, J=2.0 Hz, 1H), 7.98 (dd, J=9.8, 3.1 Hz, 1H), 7.66 (dd, J=8.9, 5.2 Hz, 1H), 7.15 (ddd, J=8.9, 7.9 3.1, 1H); HPLC-MS (ammonium acetate) [M+H]⁺=259.0.

### Example 130

### 1-(3-Chlorobenzylideneamino)guanidine Hydrochloride (4011)

3-Chlorobenzaldehyde (2.0 mmol, 281 mg) and aminoguanidine hydrochloride (2.0 mmol, 220 mg) were used according to GP3 to give the title compound (4011) as a powder (370 mg, 79%). ¹H NMR (CD₃OD) δ 8.11 (s, 1H), 7.92 (s, 1H), 7.68 (d, J=6.5 Hz, 1H), 7.43 (m, 2H); HPLC-MS (ammonium acetate) [M+H]⁺=197.1.

### Example 131

### 1-(3-Fluorobenzylideneamino)guanidine Hydrochloride (4012)

3-Fluorobenzaldehyde (2.0 mmol, 248 mg) and aminoguanidine hydrochloride (2.0 mmol, 220 mg) were used according to GP3 to give the title compound (4012) as a powder (230 mg, 53%). ¹H NMR (CD₃OD) δ 8.14 (s, 1H), 7.66 (d, J=9.9 Hz, 1H), 7.55 (d, J=7.6 Hz, 1H), 7.45 (dd, J=13.8, 7.7 Hz, 1H), 7.17 (m, 1H); HPLC-MS (ammonium acetate) [M+H]⁺=181.1.

### Example 132

### 1-(2,3,4-Trimethoxybenzalideneamino)guanidine Hydrochloride (4013)

2,3,4-Trimethoxybenzaldehyde (2.0 mmol, 392 mg) and aminoguanidine hydrochloride (2.0 mmol, 220 mg) were used according to GP3 to give the title compound (4013) as a powder (330 mg, 57%). ¹H NMR (CD₃OD) δ 8.34 (s, 1H), 7.78 (d, J=8.9 Hz, 1H), 6.86 (d, J=9.0 Hz, 1H), 3.92 (s, 3H), 3.90 (s, 3H), 3.84 (s, 3H); HPLC-MS (ammonium acetate) [M+H]⁺=253.1.

### Example 133

### 1-(3,5-Bistrifluoromethylbenzylideneamino)guanidine Hydrochloride (4014)

3,5-Bistrifluoromethylbenzaldehyde (2.0 mmol, 484 mg) and aminoguanidine hydrochloride (2.0 mmol, 220 mg) were used according to GP3 to give the title compound (4014) as a powder (360 mg, 54%). ¹H NMR (CD₃OD) δ 8.47 (s, 2H), 8.30 (s, 1H), 8.02 (s, 1H); HPLC-MS (ammonium acetate) [M+H]⁺=299.0.

### Example 134

### 1-(5-Bromo-2,4-dimethoxybenzylideneamino)guanidine Hydrochloride (4015)

5-Bromo-2,4-dimethoxybenzaldehyde (2.0 mmol, 490 mg) and aminoguanidine hydrochloride (2.0 mmol, 220 mg) were used according to GP3 to give the title compound (4015) as a powder (450 mg, 67%). ¹H NMR (CD₃OD) δ 8.34 (s, 1H), 8.20 (s, 1H), 6.69 (s, 1H), 3.95 (s, 3H), 3.93 (s, 3H); HPLC-MS (ammonium acetate) [M+H]⁺=303.0.

### Example 135

### 1-[(5-(2-(Trifluoromethyl)phenyl)-furan-2-yl)-methyleneamino]guanidine Hydrochloride (2616)

5-(2-(Trifluoromethyl)phenyl)-2-furancarboxaldehyde (2.0 mmol, 480 mg) and aminoguanidine hydrochloride (2.0 mmol, 220 mg) were used according to GP8. The crude material was purified on the CombiFlash using method CF6 to give the title compound (137FB59-8-HCl) as a pale yellow powder (318 mg, 48%) in a 9:1 mixture of two isomers. Major isomer: ¹H NMR (CD₃OD) δ 8.07 (s, 1H), 7.83 (d, J=8.0 Hz, 1H), 7.78 (dd, J=8.0, 0.6 Hz, 1H), 7.67 (t, J=7.7 Hz, 1H), 7.54 (t, J=7.6 Hz, 1H), 7.05 (d, J=3.7 Hz, 1H), 6.82 (d, J=3.6 Hz, 1H); Minor isomer: ¹H NMR (CD₃OD) δ 7.84 (d, J=8.0 Hz, 1H), 7.79 (d, J=8.0 Hz, 1H), 7.72 (t, J=7.4 Hz, 1H), 7.61 (t, J=7.6 Hz, 1H), 7.50 (s, 1H), 7.25 (d, J=3.7 Hz, 1H), 6.86 (d, J=3.7 Hz, 1H); HPLC-MS (ammonium bicarbonate) [M+H]⁺=297.3 (both isomers co-eluted).

Testing of Chemical Compounds

### Example 136

### Receptor Selection and Amplification Technology Assay

The functional receptor assay, Receptor Selection and Amplification Technology (R-SAT), was used to investigate the pharmacological properties of known and novel NPFF agonists. R-SAT is disclosed in U.S. Pat. Nos. 5,707,798, 5,912,132, and 5,955,281, all of which are hereby incorporated herein by reference in their entirety, including any drawings.

Briefly, NIH3T3 cells were grown in 96 well tissue culture plates to 70-80% confluence. Cells were transfected for 16-20 h h with plasmid DNAs using Polyfect (Qiagen Inc.) as per manufacturer's protocols. R-SAT's were generally performed with 40 ng/well of receptor and 20 ng/well of β-galactosidase plasmid DNA. All receptor and G-protein constructs used were in the pSI-derived mammalian expression vector (Promega Inc) as described previously. The NPFF receptor gene was amplified by PCR from testes cDNA using oligodeoxynucleotide primers based on the published sequence (GenBank Accession # AF257210). For large-scale transfections, cells were transfected for 16-20 h, then trypsinized and frozen in DMSO. Frozen cells were later thawed, plated at ˜20,000 cells per well of a 96 half-area well plate that contained drug. With both methods, cells were then grown in a humidified atmosphere with 5% ambient CO₂ for five days. Media was then removed from the plates and marker gene activity was measured by the addition of the β-galactosidase substrate o-nitrophenyl β-D-galactopyranoside (ONPG, in PBS with 0.5% NP-40). The resulting colorimetric reaction was measured in a spectrophotometric plate reader (Titertek Inc.) at 420 nm. All data were analyzed using the computer program XLFit (IDBSm). Efficacy is the percent maximal response compared to the maximum response elicited by a control compound (e.g. NPFF in the case of NPFF2). pEC₅₀ is the negative of the log(EC₅₀), where EC50 is the calculated concentration in molar that produces a 50% maximal response.

These experiments have provided a molecular profile, or fingerprint, for each of these agents across the most meaningful receptors, the NPFF1 and NPFF2 receptor subtypes. As can be seen in Table 1, certain compounds selectively activate NPFF2 receptors relative to NPFF1 receptors.

TABLE 1

| | NPFF1 | | NPFF2 | |
|---|---|---|---|---|
| Compound ID | pEC₅₀ | % Efficacy | pEC₅₀ | % Efficacy |
| 1001 | NT | NT | ND | 49 |
| 1002 | NT | NT | 6.2 | 41 |
| 1004 | NT | NT | ND | 106 |
| 1005 | NT | NT | 5.5 | 70 |
| 1006 | NT | NT | ND | 53 |
| 1007 | NT | NT | ND | 102 |
| 1008 | NT | NT | 5.4 | 105 |
| 1010 | NT | NT | 5.2 | 51 |
| 1011 | NT | NT | 5.0 | 47 |
| 1012 | NT | NT | 5.2 | 51 |
| 1013 | NT | NT | 5.2 | 53 |
| 1014 | NT | NT | 5.0 | 97 |
| 1016 | NT | NT | ND | 68 |
| 1017 | NT | NT | ND | 71 |
| 2002 | NT | NT | 5.6 | 39 |
| 2003 | NT | NT | 5.8 | 49 |
| 2004 | NT | NT | 5.0 | 38 |
| 2006 | NT | NT | 6.1 | 51 |
| 3005 | NA | 3 | 5.1 | 59 |
| 3006 | NA | 14 | 6.1 | 47 |
| 3007 | 5.1 | 24 | 5.2 | 84 |
| 3012 | NA | 0 | 6.6 | 63 |
| 3015 | 6.0 | 26 | 5.9 | 70 |
| 3016 | NA | 16 | 5.8 | 74 |
| 3017 | NA | 17 | 5.2 | 80 |
| 3018 | NA | 13 | 5.3 | 54 |
| 3019 | NA | 12 | 5.6 | 86 |
| 3020 | 5.4 | 41 | 5.9 | 115 |
| 3021 | 5.0 | 32 | 5.6 | 70 |
| 3022 | NA | 13 | 5.9 | 55 |
| 3023 | 6.3 | 29 | 5.9 | 22 |
| 3024 | 6.0 | 41 | 6.4 | 99 |
| 3025 | 5.5 | 28 | 5.7 | 101 |
| 3027 | NA | 5 | 5.9 | 63 |
| 3028 | NA | 9 | 5.9 | 23 |
| 3029 | NA | −1 | 5.3 | 32 |
| 3030 | NA | 7 | 5.2 | 22 |
| 3032 | 4.9 | 36 | 5.4 | 78 |
| 1045 | 5.4 | 17 | 6.1 | 64 |
| 3035 | 5.5 | 81 | 5.7 | 69 |
| 2007 | NA | 5 | 5.5 | 29 |
| 2009 | NA | 9 | 5.1 | 54 |
| 2010 | NA | 7 | 5.9 | 53 |
| 2012 | NA | 9 | 5.8 | 56 |
| 2013 | NA | 4 | 6.1 | 34 |
| 2014 | NA | 8 | 5.7 | 35 |
| 2018 | NA | 13 | 5.5 | 53 |
| 2020 | 5.9 | 24 | 6.3 | 31 |
| 2025 | 5.4 | 51 | 5.4 | 87 |
| 2026 | NA | 9 | 5.1 | 58 |
| 2028 | 5.8 | 37 | 5.9 | 68 |
| 3093 | NA | 11 | 5.8 | 89 |
| 2030 | NA | 6 | 5.6 | 62 |
| 2031 | NA | 1 | NA | 16 |
| 2032 | NA | 8 | 4.9 | 33 |
| 2033 | NA | 7 | 5.0 | 33 |
| 2034 | 5.4 | 26 | 5.6 | 105 |
| 2035 | 5.4 | 45 | 6.2 | 114 |
| 3099 | NA | 8 | 5.8 | 60 |
| 2038 | NA | 9 | 5.4 | 77 |
| 2040 | NA | 18 | 5.1 | 65 |
| 2042 | NT | NT | 6.1 | 60 |
| 2043 | NT | NT | 5.7 | 47 |
| 2044 | NT | NT | 5.4 | 45 |
| 2054 | NA | 21 | 5.0 | 96 |
| 2056 | NA | 6 | 6.3 | 73 |
| 3036 | 5.9 | 84 | 6.2 | 82 |
| 2058 | 6.0 | 25 | 6.3 | 60 |
| 4002 | 5.4 | 20 | 5.6 | 80 |
| 4003 | 4.9 | 41 | 5.2 | 41 |
| 4004 | NA | 15 | 5.0 | 49 |
| 4005 | 5.0 | 89 | 5.3 | 49 |
| 4006 | 5.4 | 78 | 5.7 | 53 |
| 3037 | 6.1 | 39 | 5.5 | 59 |
| 3038 | 6.0 | 72 | 5.9 | 65 |
| 3039 | 6.4 | 43 | 6.3 | 46 |
| 3040 | 5.8 | 48 | 5.7 | 70 |
| 3041 | 5.0 | 30 | 5.0 | 53 |
| 3043 | 6.0 | 60 | 6.0 | 92 |
| 2059 | 5.0 | 31 | 5.3 | 79 |
| 2060 | 5.4 | 26 | 5.4 | 48 |
| 2063 | 5.5 | 36 | 5.6 | 78 |
| 2065 | NA | 9 | 5.5 | 45 |
| 2068 | NA | 13 | 6.0 | 61 |
| 2069 | NA | 8 | 5.5 | 40 |
| 2072 | NA | 11 | 5.7 | 50 |
| 2073 | NA | 13 | 5.8 | 51 |
| 2074 | 5.0 | 19 | 5.6 | 77 |
| 2075 | 5.9 | 24 | 5.6 | 65 |
| 2077 | 6.4 | 21 | 5.5 | 61 |
| 4008 | 5.4 | 47 | 5.2 | 64 |
| 4015 | 5.1 | 37 | 5.7 | 64 |

NA = No activity detectable at the highest dose tested (20 μM)
NT = Not tested
ND = Not determined
Efficacy is relative to endogenous ligand

### Example 137

### CCI/Thermal Hyperalgesia

Rats were anesthetized with isoflurane under aseptic and heated conditions. The left quadriceps was shaved and scrubbed thoroughly with an iodine solution. The sciatic nerve was exposed at the level of the sciatic notch distally to the sciatic trifurcation. The nerve was very carefully freed from the underlying muscle and connective tissue without causing trauma to the nerve itself. Using 4-0 chromic catgut suture material, four semi-loose ligatures were tied around the sciatic nerve starting at the most proximal level, next to the sciatic notch, spaced roughly 1 mm apart and ending proximal to the sciatic trifurcation. Under magnification the ligatures were tightened until a slight twitch was observed in the animals left paw or musculature surrounding the nerve. The muscular incision was closed with 4-0 silk suture material and the skin was stapled with wound clips. The animals were closely observed until they recovered completely from the anesthetic. The surgery was the same for the hyperalgesia and allodynia experiments.

For hyperalgesia testing, rats were placed in a tinted plastic box on top of a clear glass, temperature-regulated floor maintained at 31° C.±1° C. The floor contained a focal radiant heat source (halogen projection lamp CXL/CXP, 50 W, 8v, USHIO, Tokyo). The heat source was moveable beneath the glass and had a radiant beam of approximately 3 mm in diameter that could be positioned under the plantar surface of the rat hind paw.

To initiate the test, rats were placed in the tinted boxes and allowed 10-20 minutes to acclimate to the new environment. The radiant heat source was then positioned under the plantar surface of the hind paw. Upon activation of the heat source, a timer was simultaneously triggered. Upon reflex movement of the hind paw, a motion sensor was activated stopping the timer and inactivating the heat source. The thermal source was adjusted so that the average response latency for an uninjured animal is no greater than 20 seconds. Each rat had two days of pre-operative baseline latency measurements in which the left rear hind paw plantar surface was measured three to four times. Two to three left postoperative baseline latency measurements were taken before and after the treatment was given. Postoperative day 2 and 4 measurements yielded the greatest degree of hyperalgesia and thus were utilized in this assay. Each animal was tested twice with at least 48 hours separating each test.

Thermal hyperalgesia developed in the surgical-treated left paw as evidenced by a decrease in paw withdrawal latencies to a thermal stimulus. The maximal hyperalgesia occurred on post-operative days 2 through 4. Paw withdrawal latencies on the surgically-treated left side gradually returned to baseline levels over the course of 5 to 12 days post-surgery. The surgically untreated right paw was not significantly affected by surgery as evidenced by similar paw withdrawal latencies throughout the 12 days of testing.

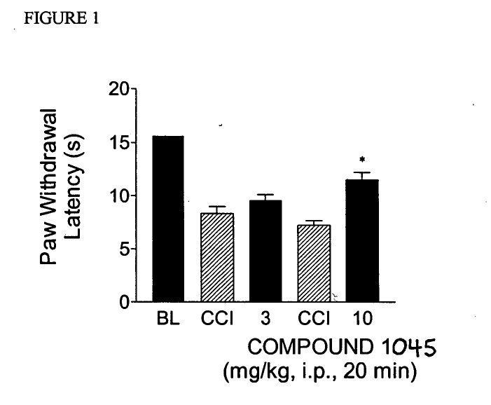

Vehicle administration in each group did not alter the thermal hyperalgesia. In contrast, the NPFF2 selective agonist Compound 1 dose dependently reversed the thermal hyperalgesia in these surgically treated rats; reaching statistical significance at the 10 mg/kg dose level (FIG. 1).

### Example 138

### CCI/Tactile Allodynia

Following the same surgical procedure described above, the onset and duration of significant mechanical allodynia post CCI surgery is approximately 10-14 days and lasts for roughly two months. Within this allodynic time frame, and for each specific allodynia experiment, pre and post drug administration measurements were taken with seven von Frey hairs which are designated by [log (10* force required to bend hair, mg)] and ranged from 2-26 grams (#'s 4.31-5.46). Each hair was pressed perpendicularly against the left injured plantar mid-hind paw surface with sufficient force to cause a slight bending, and was held for 6-8 seconds starting with the thinnest gauged hair and working up to the thickest. A positive response was recorded when the injured paw was sharply withdrawn, and this response was confirmed as positive by testing the next thickest gauged hair for the same response. Only when a response was seen twice was the score accepted. If the maximum gram force of 26 was reached without a response, this was considered the peak threshold cutoff for allodynic behavior and the score was recorded. Animals were considered allodynic when the post surgery baseline measurements were 6 grams and below. Two baseline days of measurements were taken with one round of testing occurring per day. On the day of drug testing, one round of baseline measurements were taken, the appropriate pretreatment was administered i.p. and a second round of measurements were recorded. Each animal was utilized in multiple experiments, with one treatment per experiment, and an appropriate washout period in between experiments.

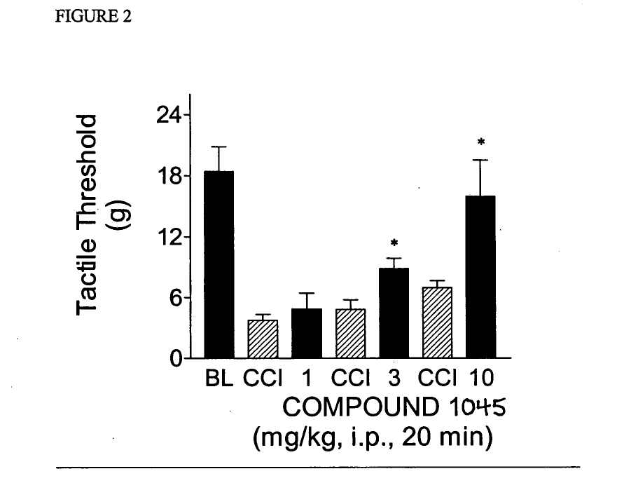

Significant tactile allodynia was seen starting on day 8 and continuing through day 35-post surgery. Assessment of tactile responsivity after administration of Compound 1 was performed within these post surgical time points. In the vehicle treated group post injury pre-treatment scores were not statistically significant from base line. Compound 1 dose dependently reversed the tactile allodynia in these surgically treated rats; reaching statistical significance at. the 3.0 and 10.0 mg/kg doses (FIG. 2).

### Example 139

### Acute Thermal Analgesia

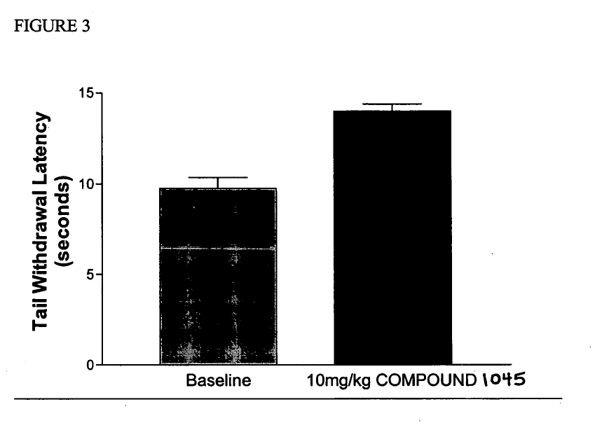

Male mice weighing approximately 20 g -30 g were acclimated to the testing apparatus. On the day of the experiment each mouse was placed in a plastic restrainer on a glass platform. A heat source was focused at the tail approximately 1 inch from the tip and from underneath the glass platform. The heat source (IR 45) was turned on and gradually increased until the mouse flicked its tail away from the heat source. The amount of time until the mouse flicked its tail was recorded. If the animal did not respond within 20 seconds, the experimenter turned off the heat and recorded this as the maximum score. One round of baseline measurements were collected. The test compound was administered and after the appropriate pretreatment interval, the procedure was repeated. The effects of Compound 1 on acute nociception are shown in FIG. 3. Compound 1 produced significant antinociception at the 10.0 mg/kg dose (FIG. 3).

### Example 140

### NPFF Receptor Binding Assay

Using the following reagents, supplies, and methods, the ability of the compounds of the invention to bind to the NPFF receptors can be readily determined in a receptor binding assay.

1. Grow NPFF receptor-transfected COS cells (or another transfected cell line that does not endogenously express the NPFF receptors may be substituted) in a suitable growth medium in 24-well culture plates.

2. Prepare radiolabeled assay solutions by mixing 245 μL of 0.25 nM [¹²⁵I]NPFF working solution with 5 μL of the following (one per solution): 50 μM unlabeled NPFF working solution, 0.25 nM [¹²⁵I]NPFF working solution, HEPES buffer only, or 50× test compound.

3. Aspirate medium from 24-well plates using a Pasteur pipet attached to a vacuum source. Do not wash cells.

4. Add 250 μL radiolabeled assay solution from step 2 to each assay well and incubate plates 60 min at room temperature (˜22° C.) on an orbital shaker at low speed.

5. Terminate the incubation by aspirating the radioactive solution with a 24-well Brandel cell harvester. Wash the wells three times with 0.5 mL ice-cold HEPES buffer using the cell harvester.

6. Aspirate the solution from the wells with a micropipettor and transfer to 12×75-mm polystyrene test tubes. Analyze with a gamma counter (Packard, Cobra II).

7. Determine specific binding and calculate the dissociation constant Kd.

### Example 142

### Other Experiments

Assessment of Intrathecally Administered NPFF in the 52° C. Water Tail Flick Test

Rats were implanted with chronically indwelling intrathecal catheters (PE-10; 7.5 cm) allowing for the delivery of compounds to the lumbar spinal cord. As a positive control, rats were treated with various doses of morphine (3, 10 and 30 μg). Morphine produced dose-related antinociception resulting in a calculated A₅₀ of 9.8 μg (8.1-12.0; 95% CI). Administration of NPFF (100 μg) failed to elicit antinociception.

Assessment of Intrathecally Administered Compound 1045 in the 52° C. Water Tail Flick Test

Rats were implanted with chronically indwelling intrathecal catheters (PE-10; 7.5 cm) allowing for the delivery of compounds to the lumbar spinal cord. Administration of Compound 1045 (11.6 or 115.5 μg) failed to elicit antinociception.

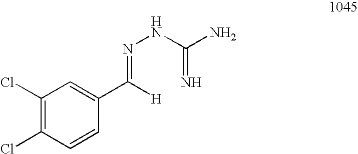

Assessment of Intrathecally Administered 1DME in the 52° C. Water Tail Flick Test

Rats were implanted with chronically indwelling intrathecal catheters (PE-10; 7.5 cm) allowing for the delivery of compounds to the lumbar spinal cord. In order to rule out the possibility that the lack of antinociception produced by NPFF was due to the degradation of the peptide we administered 1DME (a stable NPFF analog). Administration of 1DME at the doses tested (5.6, 55.6 or 556.0 μg) failed to elicit antinociception.

Effect of Systemically Administered dPQR on Compound 2616-induced Tactile Allodynia

To confirm that the pronociceptive actions of Compound 2616 were mediated via NPFF1 receptors we performed a pharmacological experiment where we administered dPQR (Dansyl-Pro-Gln-Arg, a reported NPFF antagonist, custom synthesized by Phoenix Pharmaceuticals) to rats treated with Compound 2616. Baseline paw withdrawal thresholds were obtained in naive rats. Following testing, the rats received either vehicle or Compound 2616 (10 mg/kg, i.p.). Rats were then tested 75 min post-injection and the paw withdrawal thresholds of rats that received Compound 2616 were markedly decreased as compared to those rats that received vehicle. Half of the rats that received Compound 2616 were then injected with either vehicle or dPQR (30 mg/kg, i.p.). Administration of dPQR significantly attenuated the tactile hypersensitivity elicited by Compound 2616, suggesting that the pronociceptive actions of this compounds were mediated via the NPFF1 receptor.

Assessment of Systemically Administered Compound 2616 in the Hot Plate Test

Rats were injected with either vehicle or 10 mg/kg Compound 2616 (i.p.) and then assessed for possible changes in sensitivity to a noxious thermal stimulus using the 52° C. hot plate test. Compound 2616 produced a significant reduction in the hot plate latency as compared to vehicle-treated rats, indicating the presence of thermal hyperalgesia.

Assessment of Intracerebroventricularly Administered NPFF on Barrel Rotations

Following administration of Compound 3093 and Compound 3099 (30 mg/kg, i.p.), rats showed one or more of the following behaviors: immobility and staring, ataxia, splayed hind limbs, body swaying, lying on one side with spastic limb abduction and body distortions. These behaviors typically precede “barrel-rolling” seizures.

It has been reported that ICV administration of NPFF (60 μg) elicits barrel-rotation (Panula, P., A. A. Aarnisalo, and K. Wasowicz, “Neuropeptide F F, a mammalian neuropeptide with multiple functions,” Prog Neurobiol, 1996. 48(4-5): p. 461-87). Given that this is the only mention in the literature with respect to NPFF and barrel-rotation, we attempted to replicate this effect using naive rats implanted with ICV cannula. In short, 0 out of 3 rats, 1 out of 2 rats, and 3 out of 5 rats demonstrated barrel rolling seizures following ICV administration of 60, 120 and 150 μg of NPFF, respectively.

### Example 143

### Formalin Flinching

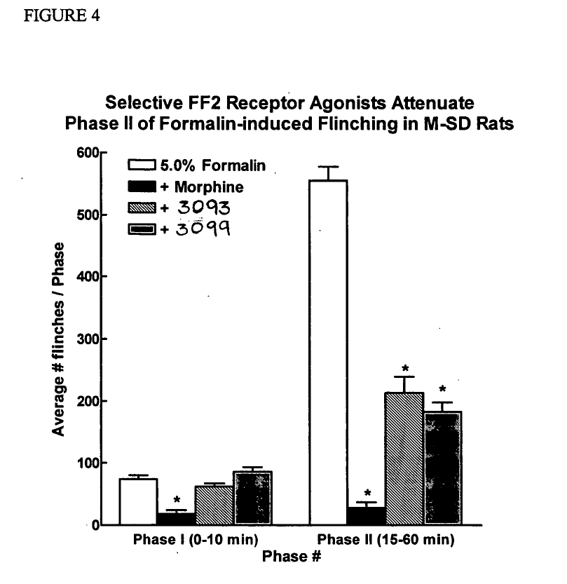

Naive male Sprague-Dawley rats (175-200 g) were injected with a test compound followed by an injection of 50 μl of a 5.0% formalin solution into the dorsal surface of a hind paw and then placed in individual plastic cages for observation. The number of nociceptive responses (i.e., paw flinches/licks/bites) was counted for a period of 60 min following formalin injection. Rats were treated with vehicle or with 10 mg/kg (i.p.) of either morphine, Compound 3093 or Compound 3099. Compounds were administered 15 min prior to formalin injection. The results are depicted in FIG. 4.

Assessment of Systemically Administered Compound 3099 in the Formalin Model

A model of tonic pain was created in rats by administering an injection of 5.0% formalin solution (50 μl) into the dorsal surface of a hind paw and then placing the rat in an individual plastic cage for observation. Paw flinches/licks/bites are counted for a period of 60 min. Rats received either vehicle or Compound 3099 (10 mg/kg, i.p.) 15 min prior to the formalin injection. Compound 3099 was inactive across phase I (0-10 min post-formalin injection), suggesting that this NPFF2 receptor selective compound is not acutely analgesic. This finding is consistent with our previous data. In contrast, across phase II (15-60 min post-formalin injection), Compound 3099 markedly attenuated (67.1% inhibition) formalin-induced flinching. This finding suggests that selective NPFF2 receptor agonists may be efficacious in states of chronic pain (i.e., neuropathic and/or inflammatory).

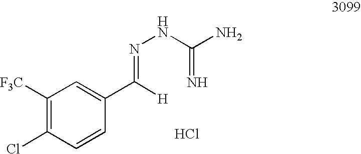

Assessment of Systemically Administered Compound 3093 in the Formalin Model

A model of tonic pain was created in rats by administering an injection of 5.0% formalin solution (50 μl) into the dorsal surface of a hind paw and then placing the rat in an individual plastic cage for observation. Paw flinches/licks/bites are counted for a period of 60 min. Rats received either vehicle or Compound 3093 (10 mg/kg, i.p.) 15 min prior to the formalin injection. Compound 3093 was inactive across phase I (0-10 min post-formalin injection), suggesting that this NPFF2 receptor selective compound is not acutely analgesic. This finding is consistent with our previous data. In contrast, across phase II (15-60 min post-formalin injection), Compound 3093 markedly attenuated (˜62.1% inhibition) formalin-induced flinching. This finding suggests that selective NPFF2 receptor agonists may be efficacious in states of chronic pain (i.e., neuropathic and/or inflammatory).

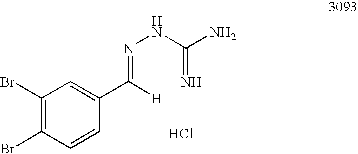

### Example 144

### Carrageenan-induced Thermal Hyperalgesia

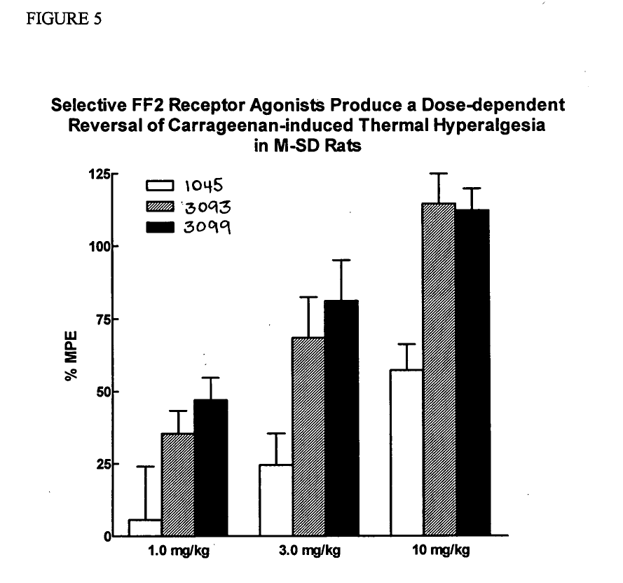

Naive male Sprague-Dawley rats (175-200 g) were assessed for their responsiveness to a noxious thermal stimulus. Response latencies were measured using the hot plate test. Rats were placed in a plexiglass enclosure on a thermostatically controlled metal plate maintained at 52° C. The time elapsed until the animal demonstrated an obvious nociceptive response (i.e., jumping, licking, stomping, elevating a hind paw) was measured. Following testing, an animal model of acute inflammatory pain was created by injecting 100 μl of 2% λ-carrageenan ion to a hind paw. Three hours after carrageenan injection, hot plate latencies were again obtained. A significant reduction in the hot plate latency was interpreted as the presence of thermal hyperalgesia. Rats were injected with compound or vehicle and then tested at various time-points following drug administration. Data were converted to % Maximum Possible Effect (% MPE) by the formula, % MPE=((test-post-inflammatory)/(naive-post-inflammatory))*100, where the test score is the hot plate latency obtained after compound administration, the post-inflammatory score is the average response obtained 3 hr post-carrageenan, and the naive score is the average response obtained prior to manipulation. Additionally, paw thickness was measured (with a micrometer) following testing in order to quantify edema. Although none of the compounds tested reversed carrageenan-induced edema formation (data not shown), these compounds produced a dose-related reversal of carrageenan-induced thermal hyperalgesia. The results are shown in FIG. 5.

Assessment of Systemically Administered Compound 1045 in Carrageenan Model

Rats were injected (i.paw.) with 100 μl of 2% carrageenan or vehicle (dH₂O) in order to produce a state of acute inflammatory pain. Following 3 hours after carrageenan, but not vehicle, administration rats demonstrated a significant increase in sensitivity to noxious thermal stimulation (i.e., decreases in the hot plate latencies). Rats were then treated with various doses of Compound 1045 (1, 3 and 10 mg/kg, i.p.) and hot plate latencies were tested across a period of 3 hours. Compound 1045 produced a dose-related reversal of thermal hyperalgesia in the carrageenan-treated rats. This compound achieved a maximum efficacy of 57.6% with a calculated A₅₀ of 7.8 mg/kg (3.9-16.0; 95% CI). Administration of Compound 1045 (10 mg/kg) to vehicle-treated rats did not significantly alter sensitivity to noxious thermal stimulation, i.e., not analgesic. This compound did not significantly alter edema formation in the hind paw produced by carrageenan.

Additionally, following administration of the hydrochloride salt of Compound 1045 (10 mg/kg, i.p.), rats demonstrated writhing behavior and appeared lethargic. These effects persisted between 15 and 20 minutes. These effects were not observed in rats that received doses less than 10 mg/kg.

Assessment of Systemically Administered Compound 3093 in Carrageenan Model

Rats were injected (i.paw.) with 100 μl of 2% carrageenan or vehicle (dH₂O) in order to produce a state of acute inflammatory pain. Following 3 hours after carrageenan, but not vehicle, administration rats demonstrated a significant increase in sensitivity to noxious thermal stimulation (i.e., decreases in the hot plate latencies). Rats were then treated with various doses of Compound 3093 (1, 3 and 10 mg/kg, i.p.) and hot plate latencies were tested across a period of 3 hours. Compound 3093 produced a dose-related reversal of thermal hyperalgesia induced by 2% carrageenan. The peak effect for Compound 3093 was observed at 30-60 min after administration and the calculated A₅₀ was 1.6 mg (1.1-2.3; 95% CI). Compound 3093 (10 mg/kg) did not significantly alter the hot plate latencies in the vehicle-treated rats.

Assessment of Systemically Administered Compound 3099 in Carrageenan Model

Rats were injected (i.paw.) with 100 μl of 2% carrageenan or vehicle (dH₂O) in order to produce a state of acute inflammatory pain. Following 3 hours after carrageenan, but not vehicle, administration rats demonstrated a significant increase in sensitivity to noxious thermal stimulation (i.e., decreases in the hot plate latencies). Rats were then treated with various doses of Compound 3099 (1, 3 and 10 mg/kg, i.p.) and hot plate latencies were tested across a period of 3 hours. Compound 3099 produced a dose-related reversal of thermal hyperalgesia induced by 2% carrageenan. The peak effect for Compound 3099 was observed at 30-60 min after administration and the calculated A₅₀ was 1.1 mg (0.7-1.6; 95% CI). Compound 3099 (10 mg/kg) did not significantly alter the hot plate latencies in the vehicle-treated rats.

### Example 145

### L₅/L₆ SNL-induced Tactile Allodynia

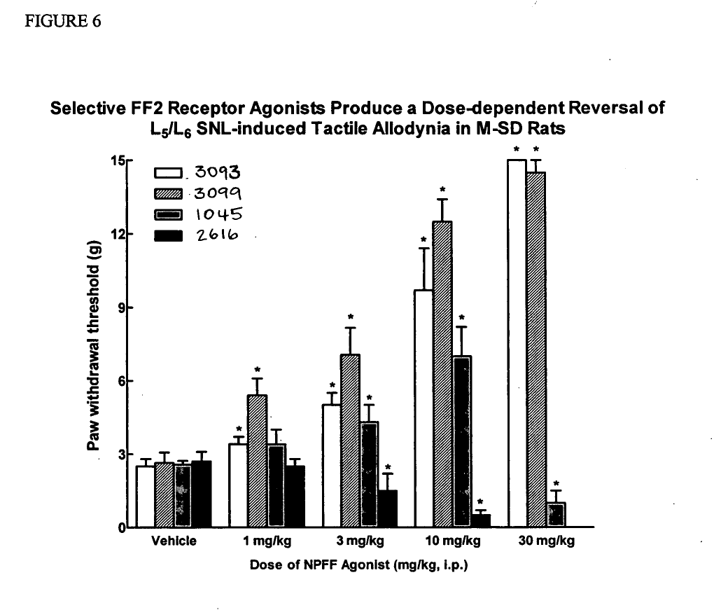

This model of neuropathic pain was developed by Kim and Chung (Kim S H, Chung J M., “An experimental model for peripheral neuropathy produced by segmental spinal nerve ligation in the rat,” Pain, 1992 September; 50(3):355-63). This model requires the ligation of the L₅ and L₆ spinal nerves between the spinal cord and the entry point into the sciatic nerve. Seven to fourteen days following SNL surgery rats will be reassessed for their response thresholds to mechanical stimuli. For the assessment of paw withdrawal thresholds rats were allowed to acclimate within plexiglass enclosures for approximately 20 min. A series of calibrated von Frey filaments (1.56-15.0 g, logarithmically spaced) were applied to the plantar aspect of the injured hind paw until a response was elicited. Paw withdrawal thresholds to probing were determined according to a previously described method (Chaplan S R, Bach F W, Pogrel J W, Chung J M, Yaksh T L., “Quantitative assessment of tactile allodynia in the rat paw,” J Neurosci Methods, 1994 July;53(1):55-63). Paw withdrawal thresholds were determined to the nearest 0.1 g before surgery, then before and at multiple time points following compound administration. A significant reduction in the paw withdrawal threshold was interpreted as the presence of tactile allodynia. The results are shown in FIG. 6.

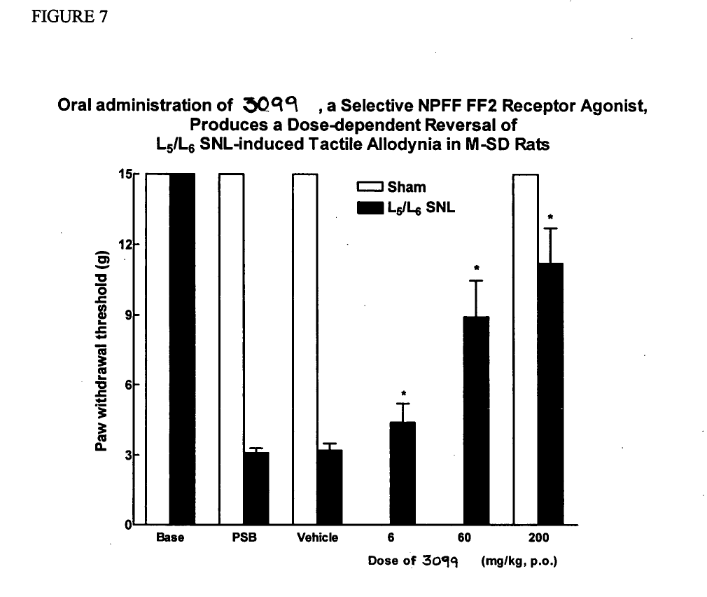

These data indicate that the selective FF2 receptor agonists (such as Compounds 3093 and 3099) dose-dependently reverse tactile allodynia induced by ligation of the L₅ and L₆ spinal nerves. Moreover, compounds with greater activity at FF1 receptors (such as Compounds 1045 and 2616) either demonstrate very little efficacy or potentiate tactile allodynia. Compound 2616 also produced tactile allodynia in sham-operated rats. The results are shown in FIG. 7.

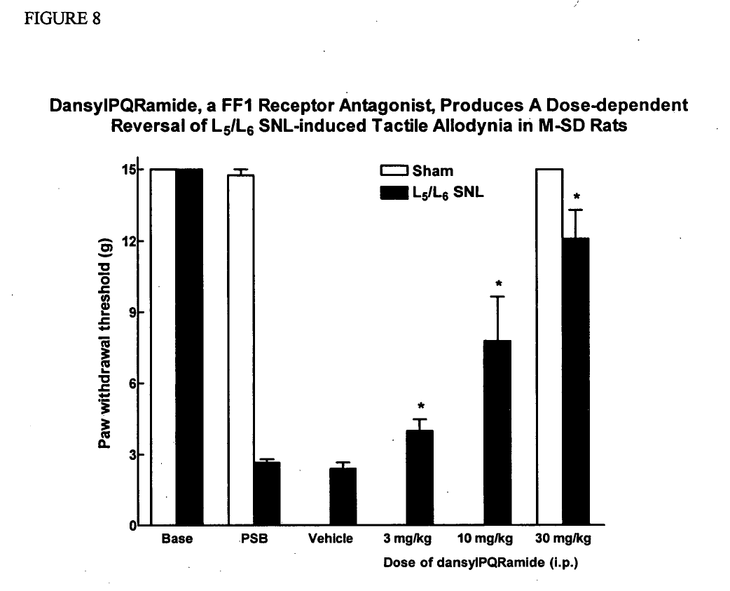

In order to study the endogenous activity of the NPFF system following injury to peripheral nerves, the activity of a FF1 receptor antagonist, dPQR, was assessed in a model of neuropathic pain. In this model, the L₅ and L₆ spinal nerves between the spinal cord and the entry point into the sciatic nerve were ligated (Kim & Chung, 1992). Seven to fourteen days following SNL surgery rats were reassessed for their response thresholds to mechanical stimuli. For the assessment of paw withdrawal thresholds rats were allowed to acclimate within plexiglass enclosures for approximately 20 min. A series of calibrated von Frey filaments (1.56-15.0 g, logarithmically spaced) were applied to the plantar aspect of the injured hind paw until a response was elicited. Paw withdrawal thresholds to probing were determined according to a previously described method (Chaplan et al., 1994). Paw withdrawal thresholds were determined to the nearest 0.1 g before surgery, then before and at multiple time points following compound administration. A significant reduction in the paw withdrawal threshold was interpreted as the presence of tactile allodynia. The results are shown in FIG. 8.

Administration of dPQR produced a dose-dependent reversal of L₅/L₆ SNL-induced tactile allodynia. These data suggest that following peripheral nerve injury there may be an inappropriate level of supraspinal FF1 receptor activation that may promote neuropathic pain.

According to the literature, spinal administration NPFF elicits acute antinociception. However, following ICV administration, NPFF results in pronociception. It has been demonstrated that FF2 receptors are located in both brain and spinal cord whereas FF1 receptors are located in brain but not in spinal cord. Taken together, these data show that the pronociceptive actions of NPFF are mediated via supraspinal FF1 receptors.

It is, therefore, demonstrated for the first time that selective FF2 receptor agonists (such as Compounds 3093 and 3099) are efficacious against inflammatory hyperalgesia and nerve injury-induced allodynia. Further, it is shown that as the activity of the compounds disclosed herein for the FF1 receptor increases, the effect on pain alleviation decreases. Additionally, administration of the FF1 agonists disclosed herein, such as Compound 2616, resulted in an increased sensitivity to innocuous tactile stimulation (i.e., tactile allodynia). This increased sensitivity was completely blocked by treatment with the FF1 antagonist dPQR. These data provide the first direct evidence for the opposing roles of supraspinal FF1 and FF2 receptors.

It is widely accepted that the role of neuropeptides in the CNS is to exert modulatory control over endogenous systems. NPFF has been proposed to modulate pain sensation, such that, under normal circumstances the opposing interplay between FF1 and FF2 receptors may be responsible for setting baseline sensory thresholds. Here it is shown that the endogenous NPFFergic system becomes increasingly active, resulting in enhanced activity of FF1 receptors at key supraspinal sites. This increased FF1 receptor activation manifests behaviorally as a state of abnormal pain. This conclusion is supported by experiments using the exogenously administered FF1 agonist, Compound 2616, in naive rats. Additional support in concept is provided by the experiments in which the actions of endogenous FF1 receptor were blocked, using dPQR (FF1 antagonist), resulting in a normalization of sensory thresholds.

Furthermore, the combination of an FF1 antagonist together with FF2 agonist blocks chronic pain in a synergistic manner. Since a) following peripheral nerve injury there appears to be an increased activity of supraspinal FF1 receptors; b) supraspinal FF1 receptors oppose the actions of supraspinal FF2 receptors and c) tactile allodynia is mediated via supraspinal mechanisms, blockade of supraspinal FF1 receptors allow for the unopposed activity of FF2 receptors to be unmasked.

Assessment of Systemically Administered Compound 1045 in the SNL Model

A model of neuropathic pain was created in rats by tight ligation of the L₅ and L₆ spinal nerves. Approximately 7-14 days following surgery rats that received the SNL, but not sham, surgery demonstrated significant increases in sensitivity to non-noxious mechanical stimulation (i.e., decreases in paw withdrawal thresholds). Rats were then treated with various doses of Compound 1045 (1, 3 and 10 mg/kg, i.p.) and paw withdrawal thresholds were tested across a period of 2 hours. Compound 1045 produced a dose-related reversal of tactile allodynia in the SNL rats. This compound achieved a maximum efficacy of 37.9%. Administration of Compound 1045 (10 mg/kg) to sham-operated rats did not significantly alter sensitivity to non-noxious mechanical stimulation.

Additionally, following administration of Compound 1045 (10 mg/kg, i.p.), rats demonstrated writhing behavior and appeared lethargic. These effects persisted between 15 and 20 minutes and were shown by both sham-operated and SNL rats. These effects were not observed in rats that received doses less than 10 mg/kg.

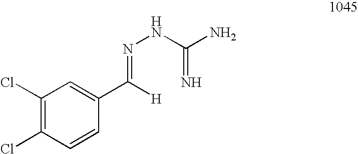

Assessment of Systemically Administered Compound 1045 (30 mg/kg) in SNL Model

In an attempt to increase the efficacy of Compound 1045 in the SNL model, we administered a dose of 30 mg/kg to SNL rats. Administration of Compound 1045 was initially efficacious at the 30 min time-point, however, by 60 min and until the end of the testing session, this compound had significantly reduced the paw withdrawal thresholds to levels below those obtained in SNL vehicle-treated rats, suggesting a potentiation of tactile allodynia.

Additionally, similar side effects were noted after 30 mg/kg Compound 1045 as were noted in the rats that received 10 mg/kg. However, these effects were more robust and of longer duration (60-90 min). A number of new side effects were noted including, ptosis, shuffling/stomping and biting of forlimbs and hindlimbs.

Assessment of Systemically Administered Compound 2616 in SNL Model

A model of neuropathic pain was created in rats by tight ligation of the L₅ and L₆ spinal nerves. Approximately 7-14 days following surgery rats that received the spinal nerve ligation (SNL), but not sham, surgery demonstrated significant increases in sensitivity to non-noxious mechanical stimulation (i.e., decreases in paw withdrawal thresholds). Rats were then treated with various doses of Compound 2616 (1, 3 and 10 mg/kg, i.p.) and paw withdrawal thresholds were tested across a period of 2.5 hours. Compound 2616 produced a dose-related potentiation of tactile allodynia in the SNL rats. Furthermore, 10 mg/kg of this compound produced a significant reduction in the paw withdrawal thresholds of sham-operated rats.

Additionally, following administration of Compound 2616 (10 mg/kg, i.p.), rats demonstrated writhing behavior and appeared lethargic. These effects persisted between 60 and 90 minutes and were shown by both sham-operated and SNL rats. These effects were not observed in rats that received doses less than 10 mg/kg.

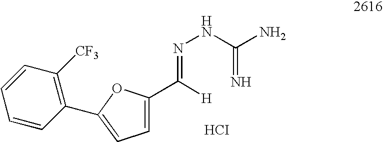

Effect of Systemically Administered dPOR in the SNL Model

A model of neuropathic pain was created in rats by tight ligation of the L₅ and L₆ spinal nerves. Approximately 7-14 days following surgery rats that received the SNL, but not sham, surgery demonstrated significant increases in sensitivity to non-noxious mechanical stimulation (i.e., decreases in paw withdrawal thresholds). Rats were then treated with various doses of dPQR (3, 10 and 30 mg/kg, i.p.) and paw withdrawal thresholds were tested across a period of 3 hours. Administration of dPQR resulted in a dose-related reversal of tactile allodynia in the SNL rats. This compound achieved a maximum efficacy of 76.7% with a calculated A₅₀ of 12.3 mg (8.0-18.9; 95%CI). Administration of dPQR (30 mg/kg, i.p.) to sham-operated rats did not significantly alter sensitivity to non-noxious mechanical stimulation. No obvious adverse side effects were observed in any of the rats that received dPQR.

Assessment of Systemically Administered Compound 3099 in the SNL Model

A model of neuropathic pain was created in rats by tight ligation of the L₅ and L₆ spinal nerves. Approximately 7-14 days following surgery rats that received the SNL, but not sham, surgery demonstrated significant increases in sensitivity to non-noxious mechanical stimulation (i.e., decreases in paw withdrawal thresholds). Rats were then treated with various doses of Compound 3099 (1, 3 and 10 mg/kg, i.p.) and paw withdrawal thresholds were tested across a period of 3 hours. The selective NPFF2 receptor agonist, Compound 3099, produced a dose-related reversal of tactile allodynia induced by L₅/L₆ SNL. The peak effect for Compound 3099 was observed at 30 min after administration and the calculated A₅₀ was 4.1 mg (3.0-5.5; 95% CI). Ptosis and lethargy were the only side effects noted in the rats that received 10 mg/kg.

Assessment of Systemically Administered Compound 3093 in the SNL Model

A model of neuropathic pain was created in rats by tight ligation of the L₅ and L₆ spinal nerves. Approximately 7-14 days following surgery rats that received the SNL, but not sham, surgery demonstrated significant increases in sensitivity to non-noxious mechanical stimulation (i.e., decreases in paw withdrawal thresholds). Rats were then treated with various doses of Compound 3093 (1, 3, 10 and 30 mg/kg, i.p.) and paw withdrawal thresholds were tested across a period of 3 hours. The selective NPFF2 receptor agonist, Compound 3093, produced a dose-related reversal of tactile allodynia induced by L₅/L₆ SNL. The peak effect for Compound 3093 was observed at 30 min after administration and the calculated A₅₀ was 6.2 mg (4.5-8.1; 95% CI).

Assessment of Systemically Administered Compound 3099 (30 mg/kg) in SNL Model

In an attempt to increase the efficacy of Compound 3099 in the SNL model, we administered a dose of 30 mg/kg (i.p.) to SNL rats. Although almost fuly efficacious in reversing SNL-induced tactile allodynia, Compound 3099 (30 mg/kg, i.p.) also produced similar effects as reported with Compound 3099. Specifically, rats demonstrated one or more of the following behaviors: immobility and staring, ataxia, splayed hind limbs, body swaying, lying on one side with spastic limb abduction and body distortions. Again, these behaviors were episodic and did not interfere with the behavioral measures. Further, these behaviors were also transient, such that, by the end of the testing period these effects appeared to have resolved.

Assessment of Orally Administered Compound 3099 in the SNL Model

A model of neuropathic pain was created in rats by tight ligation of the L₅ and L₆ spinal nerves. Approximately 14-28 days following surgery rats that received the SNL, but not sham, surgery demonstrated significant increases in sensitivity to non-noxious mechanical stimulation (i.e., decreases in paw withdrawal thresholds). Rats were then treated with various doses of Compound 3099 (6, 60 and 200 mg/kg, p.o.) and paw withdrawal thresholds were tested across a period of 3 hours. The selective NPFF2 receptor agonist, Compound 3099, produced a dose-related reversal of tactile allodynia induced by L₅/L₆ SNL. The peak effect for Compound 3099 was observed at 60-90 min after administration and the calculated A₅₀ was 50.5 mg (22.1-115.5; 95% CI).

### Example 146

### cAMP Assay

An assay was established for measuring cAMP in transiently transfected cells that takes advantage of the fact that most cells that are transfected with one gene, can be simultaneously transfected with other genes. Thus, the NPFF1 and NPFF2 receptors were transfected along with a Gs-coupled receptor (EP2) at a ratio of 5:1. In un-transfected HEK-T cells there is no response to PGE2 (agonist for EP2) at doses as high 10 μM. The cells were routinely stimulated with PGE2 at about 300 nM, which is 2× its EC₅₀ (170 nM) at EP2 receptor. Improvement was also detected in the sensitivity of the assay in some cases when cells are co-transfected with AC₅ at ½-⅕ of the amount of DNA of the Gi-coupled receptor studied.

This set up was routinely used for the transfection of HEK-T cells with NPFF1 and NPFF2 receptors. After 48 hours the cAMP assay was set up using DiscoveRx assay protocol with transfected cells in suspension in the presence of varying concentrations of the NPFF ligands and 300 nM of EP2 in white bottom plates. Cells were incubated for 15 minutes at 37° C. At the end of incubation, cells were lysed and the remainder of the assay performed as per DiscoveRx protocol.

For antagonist assays, the cells were pre-incubated with antagonists for 15 minutes at 37° C. prior to the addition of agonist and then PGE2 in order. Cells were incubated for another 15 minutes at 37° C. following which the cells were lysed and processed as per kit protocol.

R-SAT assay was conducted as set forth in

### Example 136.

The results are shown below, in Table 2. Multiple entries for a single compound denote different batches tested.

TABLE 2

| | NPFF2b | | | | | | NPFF1 | | | | | |
|---|---|---|---|---|---|---|---|---|---|---|---|---|
| | % Efficacy | | | pEC50 | | | % Efficacy | | | pEC50 | | |
| Comp'd | Mean | SD | N | Mean | SD | N | Mean | SD | N | Mean | SD | N |
| R-SAT | | | | | | | | | | | | |
| data | | | | | | | | | | | | |
| 1045 | 63.8 | 17.7 | 31.0 | 6.1 | 0.2 | 29.0 | | | | | | |
| 1045 | 55.2 | 13.6 | 31.0 | 6.3 | 0.3 | 26.0 | 6.3 | 4.8 | 6.0 | nd | | |
| 1045.HCl | 67.6 | 10.0 | 13.0 | 6.0 | 0.2 | 13.0 | 17.7 | 6.6 | 8.0 | nd | | |
| 2616 | 63.0 | 5.4 | 4.0 | 7.0 | 0.4 | 4.0 | 55.0 | 0.0 | 1.0 | 5.9 | 0.0 | 1.0 |
| 2616 | 80.4 | 13.7 | 4.0 | 7.0 | 0.1 | 4.0 | 66.3 | 16.5 | 5.0 | 6.5 | 0.1 | 5.0 |
| 2616 | 72.9 | 12.9 | 20.0 | 7.0 | 0.4 | 20.0 | 52.1 | 3.4 | 2.0 | 7.4 | 0.8 | 2.0 |
| 3093 | 85.3 | 19.3 | 6.0 | 6.2 | 0.2 | 6.0 | 10.2 | 4.5 | 3.0 | nd | | |
| 3093 | 89.9 | 15.4 | 4.0 | 5.9 | 0.0 | 4.0 | 11.7 | 0.0 | 1.0 | nd | | |
| 3099 | 102.3 | 20.4 | 7.0 | 6.5 | 0.3 | 7.0 | 15.4 | 2.6 | 3.0 | nd | | |
| 3099 | 114.2 | 27.8 | 4.0 | 6.3 | 0.2 | 4.0 | 45.8 | 0.0 | 1.0 | 5.5 | 0.0 | 1.0 |
| cAMP | | | | | | | | | | | | |
| data | | | | | | | | | | | | |
| 1045.HCl | 34 | 0 | 1 | | | | ND | | | | | |
| 2616 | 114 | 8 | 2 | 5.5 | 0.04 | 2 | 93 | 9 | 2 | 5.9 | 0.11 | 2 |
| 3093 | 78 | 24 | 4 | 5.2 | 0.31 | 4 | 23 | 9 | 4 | 4.8 | 0.1 | 2 |
| 3099 | 89 | 19 | 6 | 5.4 | 0.32 | 6 | 42 | 20 | 6 | 5.5 | 0.58 | 6 |

1. A method of identifying a compound effective in treating pain comprising contacting said compound with an NPFF2 receptor and determining whether said compound binds to said NPFF2 receptor.

2. A method of screening for a compound able to affect one or more activities of an NPFF2 receptor comprising the steps of,
a) contacting a recombinant cell with a test compound, wherein said recombinant cell comprises a recombinant nucleic acid expressing said NPFF2 receptor, provided that said cell does not have functional NPFF2 receptor expression from endogenous nucleic acid, and
b) determining the ability of said test compound to affect one or more activities of said NPFF2 receptor, and comparing said ability with the ability of said test compound to affect said one or more NPFF2 receptor activities in a cell not comprising said recombinant nucleic acid;
wherein said recombinant nucleic acid comprises an NPFF2 receptor nucleic acid selected from the group consisting of:
a) nucleic acid of SEQ ID NO:1,
b) nucleic acid encoding the amino acid SEQ ID NO:2,
c) a derivative thereof encoding said NPFF2 receptor, wherein said derivative encodes a receptor having one or more activities of said NPFF2 receptor and comprises at least 20 contiguous nucleotides which can hybridize under stringent hybridization conditions to a complement of at least 20 contiguous nucleotides of SEQ ID NO:1.

3. The method of claim 2, wherein said NPFF2 receptor nucleic acid encodes the amino acid sequence of a SEQ ID NO:2 derivative comprising at least 20 contiguous nucleotides which can hybridize under stringent hybridizations conditions to a complement of at least 20 contiguous nucleotides encoding the amino acid sequence of SEQ ID NO:2.

4. A method for treating acute and chronic pain of any type comprising contacting an organism with an effective amount of at least one compound wherein the compound activates an NPFF2 receptor subtype.

5. The method of claim 4 wherein the pain is associated with diabetes, viral infection, irritable bowel syndrome, amputation, cancer, or chemical injury.

6. A method of identifying a compound which is an agonist of an NPFF2 receptor, the method comprising:
contacting said NPFF2 receptor with at least one test compound; and
determining any increase in activity level of said NPFF2 receptor so as to identify a test compound which is an agonist of said NPFF2 receptor.

7. The method of claim 6, wherein the identified agonist activates the NPFF2 but not the NPFF1 receptor.

8. The method of claim 6, wherein the identified agonist is selective for the NPFF2 receptor.

9. A method of identifying a compound which is an agonist of an NPFF2 receptor, the method comprising:
culturing cells which express the NPFF2 receptor;
incubating the cells or a component extracted from the cells with at least one test compound; and
determining any increase in activity of the NPPF2 receptor so as to identify a test compound which is an agonist of a NPFF receptor.

10. The method of claim 7, wherein the cells of said culturing step overexpress said NPFF2 receptor.

11. A method for treating pain comprising
contacting an individual suffering from pain with an effective amount of at least one compound of Formula I or II,
whereby one or more symptoms of the pain are reduced;
wherein said compound of Formula I or II has the following structure: 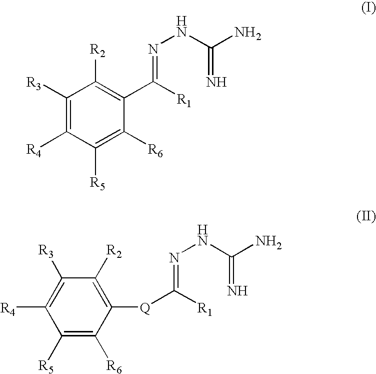
or a pharmaceutically acceptable salt, ester, amide, or prodrug thereof,
wherein
R₁ is selected from the group consisting of hydrogen, C₁-C₁₀ straight chained or branched alkyl, C₂-C₁₀ straight chained or branched alkenyl, C₂-C₁₀ straight chained or branched alkynyl, and C₃-C₁₀ cycloalkyl;
each of R₂, R₃, R₄, R₅ and R₆ is independently selected from the group consisting of hydrogen, C₁-C₁₀ straight chained or branched alkyl, C₂-C₁₀ straight chained or branched alkenyl, C₂-C₁₀ straight chained or branched alkynyl, C₃-C₁₀ cycloalkyl, substituted or unsubstituted aryl or heteroaryl, hydroxy, halogenated ether, nitro, amino, halogen, perhaloalkyl, —OR₇, —(NR₇)₂, —CN, —C(═Z)R₇, —C(═Z)OR₇, —C(═Z)N(R₇)₂, —(NR₇)—C(═Z)R₇, —(NR₇)—C(═Z)N(R₇)₂, —OC(═Z)R₇, and —SR₇
wherein Z is oxygen or sulfur; and wherein each R₇ is independently selected from the group consisting of hydrogen, C₁-C₁₀ straight chained or branched alkyl optionally substituted with an aryl or heteroaryl, C₂-C₁₀ straight chained or branched alkenyl optionally substituted with an aryl or heteroaryl, C₂-C₁₀ straight chained or branched alkynyl optionally substituted with an aryl or heteroaryl, C₃-C₁₀ cycloalkyl, C₅-C₁₀ cycloalkenyl, aryl, and heteroaryl; or
R₂ and R₃ and the carbons to which they are attached form a fused aryl, heteroaryl, C₅-C₁₀ carbocyclic or heterocyclic ring; or
R₃ and R₄ and the carbons to which they are attached form a fused aryl, heteroaryl, C₅-C₁₀ carbocyclic or heterocyclic ring; or
R₄ and R₅ and the carbons to which they are attached form a fused aryl, heteroaryl, C₅-C₁₀ carbocyclic or heterocyclic ring; or
R₅ and R₆ and the carbons to which they are attached form a fused aryl, heteroaryl, C₅-C₁₀ carbocyclic or heterocyclic ring; and
Q is selected from the group consisting of aryl, heteroaryl, C₅-C₁₀ carbocyclic or heterocyclic ring.

12 The method of claim 1, further comprising the step of identifying an individual in need of pain treatment prior to the contacting step.

13. The method of claim 11, wherein said compound of Formula I or II selectively activates the NPFF2 receptor subtype.

14. The method of claim 11, wherein the pain is associated with diabetes, viral infection, irritable bowel syndrome, amputation, cancer, inflammation or chemical injury.

15. The method of claim 11, wherein the pain is neuropathic pain.

16. The method of claim 15, wherein the subject presents hyperalgesia.

17. The method of claim 15, wherein the subject presents allodynia.

18. A method of identifying a compound that alleviates hyperalgesia or allodynia in a subject, comprising:
providing a subject suffering from hyperalgesia or allodynia with at least one compound of Formula I or II; and
determining if said at least one compound reduces hyperalgesia or allodynia in the subject;
wherein said compound of Formula I or II has the following structure: 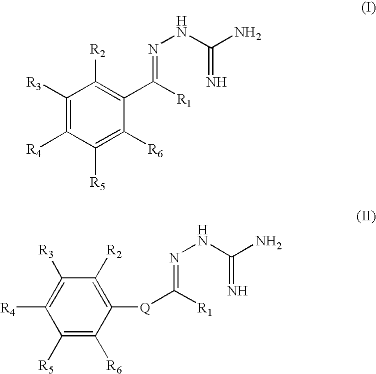
or a pharmaceutically acceptable salt, ester, amide, or prodrug thereof,
wherein
R₁ is selected from the group consisting of hydrogen, C₁-C₁₀ straight chained or branched alkyl, C₂-C₁₀ straight chained or branched alkenyl, C₂-C₁₀ straight chained or branched alkynyl, and C₃-C₁₀ cycloalkyl;
each of R₂, R₃, R₄, R₅ and R₆ is independently selected from the group consisting of hydrogen, C₁-C₁₀ straight chained or branched alkyl, C₂-C₁₀ straight chained or branched alkenyl, C₂-C₁₀ straight chained or branched alkynyl, C₃-C₁₀ cycloalkyl, substituted or unsubstituted aryl or heteroaryl, hydroxy, halogenated ether, nitro, amino, halogen, perhaloalkyl, —OR₇, —(NR₇)₂, —CN, —C(═Z)R₇, —C(═Z)OR₇, —C(═Z)N(R₇)₂, —(NR₇)—C(═Z)R₇, —(NR₇)—C(═Z)N(R₇)₂, —OC(═Z)R₇, and —SR₇
wherein Z is oxygen or sulfur; and wherein each R₇ is independently selected from the group consisting of hydrogen, C₁-C₁₀ straight chained or branched alkyl optionally substituted with an aryl or heteroaryl, C₂-C₁₀ straight chained or branched alkenyl optionally substituted with an aryl or heteroaryl, C₂-C₁₀ straight chained or branched alkynyl optionally substituted with an aryl or heteroaryl, C₃-C₁₀ cycloalkyl, C₅-C₁₀ cycloalkenyl, aryl, and heteroaryl; or
R₂ and R₃ and the carbons to which they are attached form a fused aryl, heteroaryl, C₅-C₁₀ carbocyclic or heterocyclic ring; or
R₃ and R₄ and the carbons to which they are attached form a fused aryl, heteroaryl, C₅-C₁₀ carbocyclic or heterocyclic ring; or
R₄ and R₅ and the carbons to which they are attached form a fused aryl, heteroaryl, C₅-C₁₀ carbocyclic or heterocyclic ring; or
R₅ and R₆ and the carbons to which they are attached form a fused aryl, heteroaryl, C₅-C₁₀ carbocyclic or heterocyclic ring; and
Q is selected from the group consisting of aryl, heteroaryl, C₅-C₁₀ carbocyclic or heterocyclic ring.

19. The method of claim 18, further comprising the step of identifying a subject suffering from hyperalgesia or allodynia prior to the providing step.

20. The method of claim 18, wherein said at least one compound is selective for the NPFF2 but not NPFF1 receptor.

21. The method of claim 18, wherein said hyperalgesia is thermal hyperalgesia.

22. The method of claim 18, wherein said allodynia is tactile allodynia.

23. A method of identifying a compound of Formula I or II, which is an agonist of the NPFF2 receptor, the method comprising:
contacting a NPFF2 receptor with at least one compound of Formula I or II; and
determining any increase in activity level of the NPFF2 receptor so as to identify a compound of Formula I or II, which is an agonist of the NPFF2 receptor;
wherein said compound of Formula I or II has the following structure: 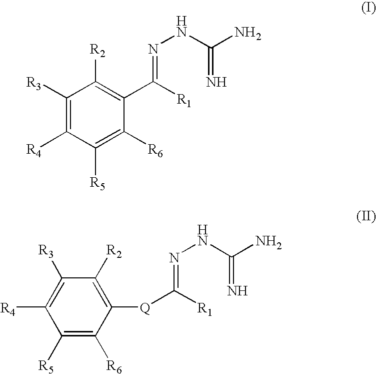
or a pharmaceutically acceptable salt, ester, amide, or prodrug thereof,
wherein
R₁ is selected from the group consisting of hydrogen, C₁-C₁₀ straight chained or branched alkyl, C₂-C₁₀ straight chained or branched alkenyl, C₂-C₁₀ straight chained or branched alkynyl, and C₃-C₁₀ cycloalkyl;
each of R₂, R₃, R₄, R₅ and R₆ is independently selected from the group consisting of hydrogen, C₁-C₁₀ straight chained or branched alkyl, C₂-C₁₀ straight chained or branched alkenyl, C₂-C₁₀ straight chained or branched alkynyl, C₃-C₁₀ cycloalkyl, substituted or unsubstituted aryl or heteroaryl, hydroxy, halogenated ether, nitro, amino, halogen, perhaloalkyl, —OR₇, —(NR₇)₂, —CN, —C(═Z)R₇, —C(═Z)OR₇, —C(═Z)N(R₇)₂, —(NR₇)—C(═Z)R₇, —(NR₇)—C(═Z)N(R₇)₂, —OC(═Z)R₇, and —SR₇
wherein Z is oxygen or sulfur; and wherein each R₇ is independently selected from the group consisting of hydrogen, C₁-C₁₀ straight chained or branched alkyl optionally substituted with an aryl or heteroaryl, C₂-C₁₀ straight chained or branched alkenyl optionally substituted with an aryl or heteroaryl, C₂-C₁₀ straight chained or branched alkynyl optionally substituted with an aryl or heteroaryl, C₃-C₁₀ cycloalkyl, C₅-C₁₀ cycloalkenyl, aryl, and heteroaryl; or
R₂ and R₃ and the carbons to which they are attached form a fused aryl, heteroaryl, C₅-C₁₀ carbocyclic or heterocyclic ring; or
R₃ and R₄ and the carbons to which they are attached form a fused aryl, heteroaryl, C₅-C₁₀ carbocyclic or heterocyclic ring; or
R₄ and R₅ and the carbons to which they are attached form a fused aryl, heteroaryl, C₅-C₁₀ carbocyclic or heterocyclic ring; or
R₅ and R₆ and the carbons to which they are attached form a fused aryl, heteroaryl, C₅-C₁₀ carbocyclic or heterocyclic ring; and
Q is selected from the group consisting of aryl, heteroaryl, C₅-C₁₀ carbocyclic or heterocyclic ring.

24. A method of identifying a compound which is an agonist of a NPFF2 receptor, the method comprising:
culturing cells that express the NPFF2 receptor;
incubating the cells with at least one compound of Formula I or II; and
determining any increase in activity of the NPPF2 receptor so as to identify a compound of Formula I or II which is an agonist of a NPFF receptor;
wherein said compound of Formula I or II has the following structure: 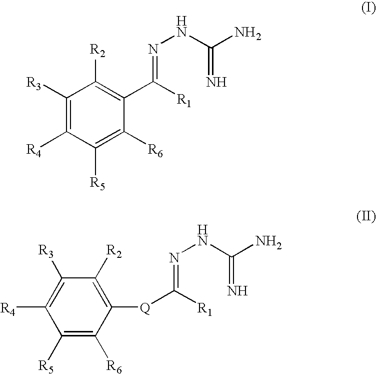
or a pharmaceutically acceptable salt, ester, amide, or prodrug thereof,
wherein
R₁ is selected from the group consisting of hydrogen, C₁-C₁₀ straight chained or branched alkyl, C₂-C₁₀ straight chained or branched alkenyl, C₂-C₁₀ straight chained or branched alkynyl, and C₃-C₁₀ cycloalkyl;
each of R₂, R₃, R₄, R₅ and R₆ is independently selected from the group consisting of hydrogen, C₁-C₁₀ straight chained or branched alkyl, C₂-C₁₀ straight chained or branched alkenyl, C₂-C₁₀ straight chained or branched alkynyl, C₃-C₁₀ cycloalkyl, substituted or unsubstituted aryl or heteroaryl, hydroxy, halogenated ether, nitro, amino, halogen, perhaloalkyl, —OR₇, —(NR₇)₂, —CN, —C(═Z)R₇, —C(═Z)OR₇, —C(═Z)N(R₇)₂, —(NR₇)—C(═Z)R₇, —(NR₇)—C(═Z)N(R₇)₂, —OC(═Z)R₇, and —SR₇
wherein Z is oxygen or sulfur; and wherein each R₇ is independently selected from the group consisting of hydrogen, C₁-C₁₀ straight chained or branched alkyl optionally substituted with an aryl or heteroaryl, C₂-C₁₀ straight chained or branched alkenyl optionally substituted with an aryl or heteroaryl, C₂-C₁₀ straight chained or branched alkynyl optionally substituted with an aryl or heteroaryl, C₃-C₁₀ cycloalkyl, C₅-C₁₀ cycloalkenyl, aryl, and heteroaryl; or
R₂ and R₃ and the carbons to which they are attached form a fused aryl, heteroaryl, C₅-C₁₀ carbocyclic or heterocyclic ring; or
R₃ and R₄ and the carbons to which they are attached form a fused aryl, heteroaryl, C₅-C₁₀ carbocyclic or heterocyclic ring; or
R₄ and R₅ and the carbons to which they are attached form a fused aryl, heteroaryl, C₅-C₁₀ carbocyclic or heterocyclic ring; or
R₅ and R₆ and the carbons to which they are attached form a fused aryl, heteroaryl, C₅-C₁₀ carbocyclic or heterocyclic ring; and
Q is selected from the group consisting of aryl, heteroaryl, C₅-C₁₀ carbocyclic or heterocyclic ring.

25. The method of claim 24, wherein the identified agonist activates the NPFF2 but not the NPFF1 receptor.

26. The method of claim 24, wherein the identified agonist is selective for the NPFF2 receptor.

27. A method of identifying a compound which is an agonist of a NPFF2 receptor, the method comprising:
contacting the NPFF2 receptor with at least one compound of Formula I or II; and
determining whether said compound of Formula I or II binds to said NPFF2 receptor;
wherein said compound of Formula I or II has the following structure: 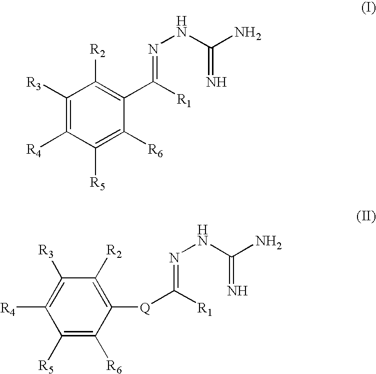
or a pharmaceutically acceptable salt, ester, amide, or prodrug thereof,
wherein
R₁ is selected from the group consisting of hydrogen, C₁-C₁₀ straight chained or branched alkyl, C₂-C₁₀ straight chained or branched alkenyl, C₂-C₁₀ straight chained or branched alkynyl, and C₃-C₁₀ cycloalkyl;
each of R₂, R₃, R₄, R₅ and R₆ is independently selected from the group consisting of hydrogen, C₁-C₁₀ straight chained or branched alkyl, C₂-C₁₀ straight chained or branched alkenyl, C₂-C₁₀ straight chained or branched alkynyl, C₃-C₁₀ cycloalkyl, substituted or unsubstituted aryl or heteroaryl, hydroxy, halogenated ether, nitro, amino, halogen, perhaloalkyl, —OR₇, —(NR₇)₂, —CN, —C(═Z)R₇, —C(═Z)OR₇, —C(═Z)N(R₇)₂, —(NR₇)—C(═Z)R₇, —(NR₇)—C(═Z)N(R₇)₂, —OC(═Z)R₇, and —SR₇
wherein Z is oxygen or sulfur; and wherein each R₇ is independently selected from the group consisting of hydrogen, C₁-C₁₀ straight chained or branched alkyl optionally substituted with an aryl or heteroaryl, C₂-C₁₀ straight chained or branched alkenyl optionally substituted with an aryl or heteroaryl, C₂-C₁₀ straight chained or branched alkynyl optionally substituted with an aryl or heteroaryl, C₃-C₁₀ cycloalkyl, C₅-C₁₀ cycloalkenyl, aryl, and heteroaryl; or
R₂ and R₃ and the carbons to which they are attached form a fused aryl, heteroaryl, C₅-C₁₀ carbocyclic or heterocyclic ring; or
R₃ and R₄ and the carbons to which they are attached form a fused aryl, heteroaryl, C₅-C₁₀ carbocyclic or heterocyclic ring; or
R₄ and R₅ and the carbons to which they are attached form a fused aryl, heteroaryl, C₅-C₁₀ carbocyclic or heterocyclic ring; or
R₅ and R₆ and the carbons to which they are attached form a fused aryl, heteroaryl, C₅-C₁₀ carbocyclic or heterocyclic ring; and
Q is selected from the group consisting of aryl, heteroaryl, C₅-C₁₀ carbocyclic or heterocyclic ring.

28. The method of claim 21, wherein the identified compound of Formula I or II is selective for the NPFF2 receptor.

29. A compound of Formula I or II 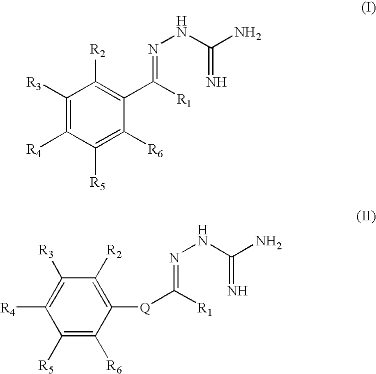
or a pharmaceutically acceptable salt, ester, amide, or prodrug thereof,
wherein
R₁ is selected from the group consisting of hydrogen, C₁-C₁₀ straight chained or branched alkyl, C₂-C₁₀ straight chained or branched alkenyl, C₂-C₁₀ straight chained or branched alkynyl, and C₃-C₁₀ cycloalkyl;
each of R₂, R₃, R₄, R₅ and R₆ is independently selected from the group consisting of hydrogen, C₁-C₁₀ straight chained or branched alkyl, C₂-C₁₀ straight chained or branched alkenyl, C₂-C₁₀ straight chained or branched alkynyl, C₃-C₁₀ cycloalkyl, substituted or unsubstituted aryl or heteroaryl, hydroxy, halogenated ether, nitro, amino, halogen, perhaloalkyl, —OR₇, —(NR₇)₂, —CN, —C(═Z)R₇, —C(═Z)OR₇, —C(═Z)N(R₇)₂, —(NR₇)—C(═Z)R₇, —(NR₇)—C(═Z)N(R₇)₂, —OC(═Z)R₇, and —SR₇
wherein Z is oxygen or sulfur; and wherein each R₇ is independently selected from the group consisting of hydrogen, C₁-C₁₀ straight chained or branched alkyl optionally substituted with an aryl or heteroaryl, C₂-C₁₀ straight chained or branched alkenyl optionally substituted with an aryl or heteroaryl, C₂-C₁₀ straight chained or branched alkynyl optionally substituted with an aryl or heteroaryl, C₃-C₁₀ cycloalkyl, C₅-C₁₀ cycloalkenyl, aryl, and heteroaryl; or
R₂ and R₃ and the carbons to which they are attached form a fused aryl, heteroaryl, C₅-C₁₀ carbocyclic or heterocyclic ring; or
R₃ and R₄ and the carbons to which they are attached form a fused aryl, heteroaryl, C₅-C₁₀ carbocyclic or heterocyclic ring; or
R₄ and R₅ and the carbons to which they are attached form a fused aryl, heteroaryl, C₅-C₁₀ carbocyclic or heterocyclic ring; or
R₅ and R₆ and the carbons to which they are attached form a fused aryl, heteroaryl, C₅-C₁₀ carbocyclic or heterocyclic ring; and
Q is selected from the group consisting of aryl, heteroaryl, C₅-C₁₀ carbocyclic or heterocyclic ring.

30. A compound of Formula III 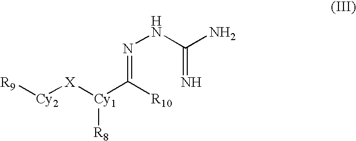
or a pharmaceutically acceptable salt, ester, amide, or prodrug thereof,
wherein
Cy₁ is selected from the group consisting of aryl, fused aryl, heteroaryl, fused heteroaryl, carbocyclic, cycloalkyl, fused heterocycle and heterocycle.
Cy₂ is selected from the group consisting of aryl, fused aryl, heteroaryl, fused heteroaryl, carbocyclic, cycloalkyl, fused heterocycle and heterocycle.
R₈ and R₉ are each present 0-6 times and are independently selected from the group consisting of hydrogen, C₁-C₈ straight chained or branched alkyl optionally substituted, C₂-C₈ straight chained or branched alkenyl optionally substituted, C₂-C₈ straight chained or branched alkynyl optionally substituted, C₃-C₈ cycloalkyl optionally substituted, carbocyclic optionally substituted, aryl optionally substituted, fused aryl optionally substituted, heteroaryl optionally substituted, fused heteroaryl optionally substituted, heterocycle optionally substituted, fused heterocycle optionally substituted, haloalkyl, halogen, —CN, —NO₂, —C(═Z)R₇, —C(═Z)OR₇, —C(═Z)N(R₇)₂, —(NR₇)₂, —(NR₇)—C(═Z)R₇, —(NR₇)—C(═Z)N(R₇)₂, —(NR₇)—S(═O)R₇, N(R₇)—S(═O)₂R₇, —OR₇, —OC(═Z)R₇, —SO₃H, —S(═O)₂N(R₇)₂, —S(═O)N(R₇)₂, —S(═O)₂R₇, —S(═O)R₇ and —SR₇,
wherein Z is oxygen or sulfur; and wherein each R₇ is as defined above;
R₁₀ is selected from the group consisting of hydrogen, C₁-C₈ straight chained or branched alkyl optionally substituted, C₂-C₈ straight chained or branched alkenyl optionally substituted, C₂-C₈ straight chained or branched alkynyl optionally substituted, C₃-C₈ cycloalkyl, aryl optionally substituted, fused aryl optionally substituted, heteroaryl optionally substituted, fused heteroaryl optionally substituted heterocycle optionally substituted, fused heterocycle optionally substituted.
X is either absent or selected from the group consisting of oxygen, sulfur, NR₇, ethylene optionally substituted, acetylene,
wherein R₇ is as defined above.

31. A method of treating neuropathic or inflammatory pain in a subject comprising contacting said subject with an antagonist of the NPFF1 receptor, wherein said antagonist is a compound of Formula I, II, or III.

32. A method of treating neuropathic or inflammatory pain in a subject comprising contacting said subject with a weak partial agonist of the NPFF1 receptor, wherein said weak partial agonist is a compound of Formula I, II, or III.

33. A method of treating neuropathic or inflammatory pain in a subject comprising contacting the subject with a combination of a compound of Formula I, II, or III, which acts as an antagonist or partial agonist to NPFF1 receptor, and another compound of Formula I, II, or III, which acts as a full agonist or a partial agonist to NPFF2 receptor.

34. A method of treating neuropathic or inflammatory pain in a subject comprising contacting the subject with a compound of Formula I, II, or III, where the compound acts as both an NPFF2 agonist and an NPFF1 antagonist.

35. A method of treating neuropathic or inflammatory pain in a subject comprising contacting the subject with a compound of Formula I, II, or III, where the compound acts as both an NPFF2 partial agonist and an NPFF1 antagonist.

36. A method of treating neuropathic or inflammatory pain in a subject comprising contacting the subject with a compound of Formula I, II, or III, where the compound acts as both an NPFF2 partial agonist and an NPFF1 partial agonist.
